SEQUENCE LISTING (308 sequences)

SEQ ID NO: 1 - protein, 11 aa, homo sapien
ISISTSGGSFR

SEQ ID NO: 2 - protein, 13 aa, homo sapien
ASLEGNLAETENR

SEQ ID NO: 3 - protein, 13 aa, homo sapien
LLEGEDAHLTQYK

SEQ ID NO: 4 - protein, 10 aa, homo sapien
NHEEEMNALR

SEQ ID NO: 5 - protein, 16 aa, homo sapien
TMQALEIELQSQLSMK

SEQ ID NO: 6 - protein, 8 aa, homo sapien
ALQLEEER

SEQ ID NO: 7 - protein, 10 aa, homo sapien
APDFVFYAPR

SEQ ID NO: 8 - protein, 8 aa, homo sapien
EKEELMLR

SEQ ID NO: 9 - protein, 8 aa, homo sapien
KENPLQFK

SEQ ID NO: 10 - protein, 12 aa, homo sapien
GQLEALQVDGGR

SEQ ID NO: 11 - protein, 10 aa, homo sapien
QEELEAALQR

SEQ ID NO: 12 - protein, 12 aa, homo sapien
SAYGGPVGAGIR

SEQ ID NO: 13 - protein, 10 aa, homo sapien
SMQDVVEDFK

SEQ ID NO: 14 - protein, 11 aa, homo sapien
TAAENEFVVLK

SEQ ID NO: 15 - protein, 9 aa, homo sapien
ALLTLADGR

SEQ ID NO: 16 - protein, 16 aa, homo sapien
DYPDFSPSVDAEAIQK

SEQ ID NO: 17 - protein, 8 aa, homo sapien
FTEILCLR

SEQ ID NO: 18 - protein, 16 aa, homo sapien
GAGTNEDALIEILTTR

SEQ ID NO: 19 - protein, 11 aa, homo sapien
GIGTDEFTLNR

SEQ ID NO: 20 - protein, 9 aa, homo sapien
MLISILTER

SEQ ID NO: 21 - protein, 9 aa, homo sapien
NTPAFLAER

SEQ ID NO: 22 - protein, 9 aa, homo sapien
SEIDLLDIR

SEQ ID NO: 23 - protein, 15 aa, homo sapien
SLGDDISSETSGDFR

SEQ ID NO: 24 - protein, 16 aa, homo sapien
LEAELGNMQGLVEDFK

SEQ ID NO: 25 - protein, 12 aa, homo sapien
LEGLTDEINFLR

SEQ ID NO: 26 - protein, 10 aa, homo sapien
LSELEAALQR

SEQ ID NO: 27 - protein, 8 aa, homo sapien
QLYEEEIR

SEQ ID NO: 28 - protein, 15 aa, homo sapien
SNMDNMFESYINNLR

SEQ ID NO: 29 - protein, 11 aa, homo sapien
TEMENEFVLIK

SEQ ID NO: 30 - protein, 10 aa, homo sapien
YEELQSLAGK

SEQ ID NO: 31 - protein, 11 aa, homo sapien
IGGIGTVPVGR

SEQ ID NO: 32 - protein, 8 aa, homo sapien
LPLQDVYK

SEQ ID NO: 33 - protein, 14 aa, homo sapien
VGAENVAIVEPSER

SEQ ID NO: 34 - protein, 12 aa, homo sapien
YADALQEIIQER

SEQ ID NO: 35 - protein, 9 aa, homo sapien
AQYEEIANR

SEQ ID NO: 36 - protein, 8 aa, homo sapien
HEISEMNR

SEQ ID NO: 37 - protein, 10 aa, homo sapien
LAELEEALQK

SEQ ID NO: 38 - protein, 9 aa, homo sapien
QLDSIVGER

SEQ ID NO: 39 - protein, 15 aa, homo sapien
QNLEPLFEQYINNLR

SEQ ID NO: 40 - protein, 14 aa, homo sapien
SFSTASAITPSVSR

SEQ ID NO: 41 - protein, 11 aa, homo sapien
TTAENEFVMLK

SEQ ID NO: 42 - protein, 15 aa, homo sapien
VSLAGACGVGGYGSR

SEQ ID NO: 43 - protein, 10 aa, homo sapien
YEELQQTAGR

SEQ ID NO: 44 - protein, 7 aa, homo sapien
IWGEDLR

SEQ ID NO: 45 - protein, 8 aa, homo sapien
SYSFDEIR

SEQ ID NO: 46 - protein, 13 aa, homo sapien
TPAGNFVTLEEGK

SEQ ID NO: 47 - protein, 8 aa, homo sapien
VYSYFECR

SEQ ID NO: 48 - protein, 13 aa, homo sapien
DYSHYYTTIQDLR

SEQ ID NO: 49 - protein, 10 aa, homo sapien
NHEEEISTLR

SEQ ID NO: 50 - protein, 17 aa, homo sapien
QSSATSSFGGLGGGSVR

SEQ ID NO: 51 - protein, 11 aa, homo sapien
SQYEVMAEQNR

SEQ ID NO: 52 - protein, 12 aa, homo sapien
AQIFANTVDNAR

SEQ ID NO: 53 - protein, 11 aa, homo sapien
QSVENDIHGLR

SEQ ID NO: 54 - protein, 8 aa, homo sapien
AQYDELAR

SEQ ID NO: 55 - protein, 10 aa, homo sapien
KVIDDTNITR

SEQ ID NO: 56 - protein, 12 aa, homo sapien
QAQEYEALLNIK

SEQ ID NO: 57 - protein, 13 aa, homo sapien
SLGSVQAPSYGAR

SEQ ID NO: 58 - protein, 8 aa, homo sapien
STFSTNYR

SEQ ID NO: 59 - protein, 13 aa, homo sapien
TVQSLEIDLDSMR

SEQ ID NO: 60 - protein, 8 aa, homo sapien
EELAYLKK

SEQ ID NO: 61 - protein, 8 aa, homo sapien
FETEQALR

SEQ ID NO: 62 - protein, 9 aa, homo sapien
IVLQIDNAR

SEQ ID NO: 63 - protein, 9 aa, homo sapien
VLDELTLAR

SEQ ID NO: 64 - protein, 9 aa, homo sapien
EYQELMNVK

SEQ ID NO: 65 - protein, 15 aa, homo sapien
AVFVDLEPTVIDEVR

SEQ ID NO: 66 - protein, 11 aa, homo sapien
TAAENEFVTLK

SEQ ID NO: 67 - protein, 11 aa, homo sapien
LDPSIFESLQK

SEQ ID NO: 68 - protein, 9 aa, homo sapien
TEVLMENFR

SEQ ID NO: 69 - protein, 17 aa, homo sapien
VEAGDVIYIEANSGAVK

SEQ ID NO: 70 - protein, 12 aa, homo sapien
AKQEELEAALQR

SEQ ID NO: 71 - protein, 12 aa, homo sapien
VDALNDEINFLR

SEQ ID NO: 72 - protein, 9 aa, homo sapien
VGFLEQQNK

SEQ ID NO: 73 - protein, 10 aa, homo sapien
GMQDLVEDFK

SEQ ID NO: 74 - protein, 10 aa, homo sapien
SGFSSISVSR

SEQ ID NO: 75 - protein, 13 aa, homo sapien
VFVEEQVYSEFVR

SEQ ID NO: 76 - protein, 17 aa, homo sapien
GLGVGFGSGGGSSSSVK

SEQ ID NO: 77 - protein, 10 aa, homo sapien
TEAESWYQTK

SEQ ID NO: 78 - protein, 14 aa, homo sapien
MVSSYVGENAEFER

SEQ ID NO: 79 - protein, 9 aa, homo sapien
GELLEAIKR

SEQ ID NO: 80 - protein, 15 aa, homo sapien
HHAAYVNNLNVTEEK

SEQ ID NO: 81 - protein, 8 aa, homo sapien
RDFGSFDK

SEQ ID NO: 82 - protein, 13 aa, homo sapien
APSWFDTGLSEMR

SEQ ID NO: 83 - protein, 9 aa, homo sapien
QDEHGFISR

SEQ ID NO: 84 - protein, 17 aa, homo sapien
EQEELLAPADGTVELVR

SEQ ID NO: 85 - protein, 9 aa, homo sapien
LEQEIATYR

SEQ ID NO: 86 - protein, 11 aa, homo sapien
TDLEMQIEGLK

SEQ ID NO: 87 - protein, 9 aa, homo sapien
VLDELTLAR

SEQ ID NO: 88 - protein, 14 aa, homo sapien
AVTEQGAELSNEER

SEQ ID NO: 89 - protein, 10 aa, homo sapien
DSTLIMQLLR

SEQ ID NO: 90 - protein, 8 aa, homo sapien
NLLSVAYK

SEQ ID NO: 91 - protein, 11 aa, homo sapien
YLAEVACGDDR

SEQ ID NO: 92 - protein, 12 aa, homo sapien
ADTLTDEINFLR

SEQ ID NO: 93 - protein, 17 aa, homo sapien
ISIGGGSCAISGGYGSR

SEQ ID NO: 94 - protein, 8 aa, homo sapien
QEIAEINR

SEQ ID NO: 95 - protein, 10 aa, homo sapien
FADLSEAANR

SEQ ID NO: 96 - protein, 10 aa, homo sapien
LGDLYEEEMR

SEQ ID NO: 97 - protein, 8 aa, homo sapien
LQEEMLQR

SEQ ID NO: 98 - protein, 11 aa, homo sapien
MALDIEIATYR

SEQ ID NO: 99 - protein, 10 aa, homo sapien
NLQEAEEWYK

SEQ ID NO: 100 - protein, 9 aa, homo sapien
VELQELNDR

SEQ ID NO: 101 - protein, 14 aa, homo sapien
ELCQGLGQPGSVLR

SEQ ID NO: 102 - protein, 15 aa, homo sapien
ELSLAGNELGDEGAR

SEQ ID NO: 103 - protein, 15 aa, homo sapien
ELTVSNNDINEAGVR

SEQ ID NO: 104 - protein, 11 aa, homo sapien
VNPALAELNLR

SEQ ID NO: 105 - protein, 10 aa, homo sapien
LKYENEVALR

SEQ ID NO: 106 - protein, 14 aa, homo sapien
SLLEGEGSSGGGGR

SEQ ID NO: 107 - protein, 12 aa, homo sapien
DAGTIAGLNVMR

SEQ ID NO: 108 - protein, 14 aa, homo sapien
ITPSYVAFTPEGER

SEQ ID NO: 109 - protein, 11 aa, homo sapien
VEIIANDQGNR

SEQ ID NO: 110 - protein, 11 aa, homo sapien
HLVDEPQNLIK

SEQ ID NO: 111 - protein, 10 aa, homo sapien
ATAVVDGAFK

SEQ ID NO: 112 - protein, 17 aa, homo sapien
EGGLGPLNIPLLADVTR

SEQ ID NO: 113 - protein, 8 aa, homo sapien
GLFIIDGK

SEQ ID NO: 114 - protein, 9 aa, homo sapien
LSEDYGVLK

SEQ ID NO: 115 - protein, 11 aa, homo sapien
QITVNDLPVGR

SEQ ID NO: 116 - protein, 8 aa, homo sapien
TDEGIAYR

SEQ ID NO: 117 - protein, 11 aa, homo sapien
LQVSQQEDITK

SEQ ID NO: 118 - protein, 11 aa, homo sapien
SLLVTELGSSR

SEQ ID NO: 119 - protein, 16 aa, homo sapien
FSLVGIGGQDLNEGNR

SEQ ID NO: 120 - protein, 9 aa, homo sapien
LSPEELLLR

SEQ ID NO: 121 - protein, 9 aa, homo sapien
NEALIALLR

SEQ ID NO: 122 - protein, 10 aa, homo sapien
QFVTATDVVR

SEQ ID NO: 123 - protein, 16 aa, homo sapien
SYELPDGQVITIGNER

SEQ ID NO: 124 - protein, 12 aa, homo sapien
VVAGVANALAHK

SEQ ID NO: 125 - protein, 13 aa, homo sapien
QEYDESGPSIVHR

SEQ ID NO: 126 - protein, 10 aa, homo sapien
FEELNADLFR

SEQ ID NO: 127 - protein, 11 aa, homo sapien
NSLESYAFNMK

SEQ ID NO: 128 - protein, 14 aa, homo sapien
SQIHDIVLVGGSTR

SEQ ID NO: 129 - protein, 11 aa, homo sapien
ALYETELADAR

SEQ ID NO: 130 - protein, 16 aa, homo sapien
SLETENSALQLQVTER

SEQ ID NO: 131 - protein, 12 aa, homo sapien
EVDEQMLNVQNK

SEQ ID NO: 132 - protein, 10 aa, homo sapien
FPGQLNADLR

SEQ ID NO: 133 - protein, 10 aa, homo sapien
LAVNMVPFPR

SEQ ID NO: 134 - protein, 10 aa, homo sapien
ISEQFTAMFR

SEQ ID NO: 135 - protein, 10 aa, homo sapien
AGFAGDDAPR

SEQ ID NO: 136 - protein, 18 aa, homo sapien
GADFLVTEVENGGSLGSK

SEQ ID NO: 137 - protein, 11 aa, homo sapien
LDIDSPPITAR

SEQ ID NO: 138 - protein, 13 aa, homo sapien
NTGIICTIGPASR

SEQ ID NO: 139 - protein, 14 aa, homo sapien
TATESFASDPILYR

SEQ ID NO: 140 - protein, 11 aa, homo sapien
KLVILEGELER

SEQ ID NO: 141 - protein, 10 aa, homo sapien
IQLVEEELDR

SEQ ID NO: 142 - protein, 10 aa, homo sapien
LVILEGELER

SEQ ID NO: 143 - protein, 7 aa, homo sapien
QLEEELR

SEQ ID NO: 144 - protein, 10 aa, homo sapien
VNHVTLSQPK

SEQ ID NO: 145 - protein, 12 aa, homo sapien
EFTPPVQAAYQK

SEQ ID NO: 146 - protein, 10 aa, homo sapien
LLVVYPWTQR

SEQ ID NO: 147 - protein, 10 aa, homo sapien
GYSFTTTAER

SEQ ID NO: 148 - protein, 13 aa, homo sapien
DAFLGSFLYEYSR

SEQ ID NO: 149 - protein, 13 aa, homo sapien
LGEYGFQNALIVR

SEQ ID NO: 150 - protein, 12 aa, homo sapien
TVMENFVAFVDK

SEQ ID NO: 151 - protein, 11 aa, homo sapien
HIVTFDGQNFK

SEQ ID NO: 152 - protein, 13 aa, homo sapien
DVFLGMFLYEYAR

SEQ ID NO: 153 - protein, 8 aa, homo sapien
FQNALLVR

SEQ ID NO: 154 - protein, 14 aa, homo sapien
VPQVSTPTLVEVSR

SEQ ID NO: 155 - protein, 10 aa, homo sapien
YEELQITAGR

SEQ ID NO: 156 - protein, 16 aa, homo sapien
TVLSGGTTMYPGIADR

SEQ ID NO: 157 - protein, 11 aa, homo sapien
DITSDTSGDFR

SEQ ID NO: 158 - protein, 14 aa, homo sapien
FIENEEQEYVQTVK

SEQ ID NO: 159 - protein, 14 aa, homo sapien
GTDVNVFNTILTTR

SEQ ID NO: 160 - protein, 11 aa, homo sapien
TPAQFDADELR

SEQ ID NO: 161 - protein, 11 aa, homo sapien
ALNSIIDVYHK

SEQ ID NO: 162 - protein, 8 aa, homo sapien
GNFHAVYR

SEQ ID NO: 163 - protein, 9 aa, homo sapien
LNYKPPPQK

SEQ ID NO: 164 - protein, 13 aa, homo sapien
TLLGDGPVVTDPK

SEQ ID NO: 165 - protein, 16 aa, homo sapien
EEVGEEAIVELVENGK

SEQ ID NO: 166 - protein, 16 aa, homo sapien
SEDFGVNEDLADSDAR

SEQ ID NO: 167 - protein, 9 aa, homo sapien
GDYPLEAVR

SEQ ID NO: 168 - protein, 16 aa, homo sapien
VTNGAFTGEISPGMIK

SEQ ID NO: 169 - protein, 15 aa, homo sapien
VVLAYEPVWAIGTGK

SEQ ID NO: 170 - protein, 10 aa, homo sapien
SSYYMIGEQK

SEQ ID NO: 171 - protein, 16 aa, homo sapien
IINEPTAAAIAYGLDR

SEQ ID NO: 172 - protein, 10 aa, homo sapien
LVNHFVEEFK

SEQ ID NO: 173 - protein, 13 aa, homo sapien
TTPSYVAFTDTER

SEQ ID NO: 174 - protein, 10 aa, homo sapien
LVNELTEFAK

SEQ ID NO: 175 - protein, 17 aa, homo sapien
AEDGSVIDYELIDQDAR

SEQ ID NO: 176 - protein, 11 aa, homo sapien
DIISDTSGDFR

SEQ ID NO: 177 - protein, 9 aa, homo sapien
QDIAFAYQR

SEQ ID NO: 178 - protein, 10 aa, homo sapien
TNQELQEINR

SEQ ID NO: 179 - protein, 11 aa, homo sapien
TPAQYDASELK

SEQ ID NO: 180 - protein, 13 aa, homo sapien
LTFDSSFSPNTGK

SEQ ID NO: 181 - protein, 11 aa, homo sapien
WTEYGLTFTEK

SEQ ID NO: 182 - protein, 13 aa, homo sapien
SSPVDLVTATDQK

SEQ ID NO: 183 - protein, 10 aa, homo sapien
EAFNMIDQNR

SEQ ID NO: 184 - protein, 11 aa, homo sapien
FTDEEVDELYR

SEQ ID NO: 185 - protein, 10 aa, homo sapien
GNFNYIEFTR

SEQ ID NO: 186 - protein, 10 aa, homo sapien
SGFSSVSVSR

SEQ ID NO: 187 - protein, 16 aa, homo sapien
VFVGGLSPDTSEEQIK

SEQ ID NO: 188 - protein, 10 aa, homo sapien
IDTIEIITDR

SEQ ID NO: 189 - protein, 16 aa, homo sapien
LFIGGLSFETTEESLR

SEQ ID NO: 190 - protein, 14 aa, homo sapien
KLEVEANNAFDQYR

SEQ ID NO: 191 - protein, 13 aa, homo sapien
LEVEANNAFDQYR

SEQ ID NO: 192 - protein, 14 aa, homo sapien
YDPPLEDGAMPSAR

SEQ ID NO: 193 - protein, 9 aa, homo sapien
MFLSFPTTK

SEQ ID NO: 194 - protein, 16 aa, homo sapien
TYFPHFDLSHGSAQVK

SEQ ID NO: 195 - protein, 15 aa, homo sapien
VGAHAGEYGAEALER

SEQ ID NO: 196 - protein, 16 aa, homo sapien
SLGLSLSGGDQEDAGR

SEQ ID NO: 197 - protein, 12 aa, homo sapien
CFIVGADNVGSK

SEQ ID NO: 198 - protein, 11 aa, homo sapien
GHLENNPALEK

SEQ ID NO: 199 - protein, 9 aa, homo sapien
GNVGFVFTK

SEQ ID NO: 200 - protein, 10 aa, homo sapien
IIQLLDDYPK

SEQ ID NO: 201 - protein, 14 aa, homo sapien
LTEENGFWYLDQIR

SEQ ID NO: 202 - protein, 9 aa, homo sapien
NIFNISLQR

SEQ ID NO: 203 - protein, 10 aa, homo sapien
YQETFNVIER

SEQ ID NO: 204 - protein, 9 aa, homo sapien
IILQIDNAR

SEQ ID NO: 205 - protein, 16 aa, homo sapien
QSGEAFVELGSEDDVK

SEQ ID NO: 206 - protein, 11 aa, homo sapien
NMINTFVPSGK

SEQ ID NO: 207 - protein, 11 aa, homo sapien
STYPPSGPTYR

SEQ ID NO: 208 - protein, 8 aa, homo sapien
WHELMLDK

SEQ ID NO: 209 - protein, 12 aa, homo sapien
APLDIPVPDPVK

SEQ ID NO: 210 - protein, 13 aa, homo sapien
EPALNEANLSNLK

SEQ ID NO: 211 - protein, 14 aa, homo sapien
IEDGNNFGVAVQEK

SEQ ID NO: 212 - protein, 10 aa, homo sapien
LEGFHTQISK

SEQ ID NO: 213 - protein, 7 aa, homo sapien
LMVMEIR

SEQ ID NO: 214 - protein, 9 aa, homo sapien
ISELDAFLK

SEQ ID NO: 215 - protein, 12 aa, homo sapien
QLVHELDEAEYR

SEQ ID NO: 216 - protein, 10 aa, homo sapien
NMQDMVEDYR

SEQ ID NO: 217 - protein, 12 aa, homo sapien
AHLVAVFNEYQR

SEQ ID NO: 218 - protein, 13 aa, homo sapien
GTITDAPGFDPLR

SEQ ID NO: 219 - protein, 15 aa, homo sapien
SLYHDISGDTSGDYR

SEQ ID NO: 220 - protein, 15 aa, homo sapien
QCANLQAAIADAEQR

SEQ ID NO: 221 - protein, 18 aa, homo sapien
AAVPSGASTGIYEALELR

SEQ ID NO: 222 - protein, 13 aa, homo sapien
GNPTVEVDLFTSK

SEQ ID NO: 223 - protein, 14 aa, homo sapien
LAQANGWGVMVSHR

SEQ ID NO: 224 - protein, 10 aa, homo sapien
IGAEVYHNLK

SEQ ID NO: 225 - protein, 15 aa, homo sapien
VNQIGSVTESLQACK

SEQ ID NO: 226 - protein, 14 aa, homo sapien
VVIGMDVAASEFFR

SEQ ID NO: 227 - protein, 12 aa, homo sapien
YISPDQLADLYK

SEQ ID NO: 228 - protein, 14 aa, homo sapien
AQIHDLVLVGGSTR

SEQ ID NO: 229 - protein, 11 aa, homo sapien
FELSGIPPAPR

SEQ ID NO: 230 - protein, 11 aa, homo sapien
NALESYAFNMK

SEQ ID NO: 231 - protein, 13 aa, homo sapien
AALEDTLAETEAR

SEQ ID NO: 232 - protein, 9 aa, homo sapien
AVFPSIVGR

SEQ ID NO: 233 - protein, 18 aa, homo sapien
VAPEEHPVLLTEAPLNPK

SEQ ID NO: 234 - protein, 16 aa, homo sapien
AYLPVNESFGFTADLR

SEQ ID NO: 235 - protein, 17 aa, homo sapien
EITENLMATGDLDQDGR

SEQ ID NO: 236 - protein, 12 aa, homo sapien
GSVSDEEMMELR

SEQ ID NO: 237 - protein, 14 aa, homo sapien
DPVQEAWAEDVDLR

SEQ ID NO: 238 - protein, 16 aa, homo sapien
THNLEPYFESFINNLR

SEQ ID NO: 239 - protein, 13 aa, homo sapien
NYSPYYNTIDDLK

SEQ ID NO: 240 - protein, 12 aa, homo sapien
GGAEQFMEETER

SEQ ID NO: 241 - protein, 14 aa, homo sapien
LPIGDVATQYFADR

SEQ ID NO: 242 - protein, 17 aa, homo sapien
NDSVVAGGGAIEMELSK

SEQ ID NO: 243 - protein, 12 aa, homo sapien
VDALMDEINFMK

SEQ ID NO: 244 - protein, 10 aa, homo sapien
NMQDLVEDFK

SEQ ID NO: 245 - protein, 9 aa, homo sapien
AYTNFDAER

SEQ ID NO: 246 - protein, 16 aa, homo sapien
GLGTDEDSLIEIICSR

SEQ ID NO: 247 - protein, 18 aa, homo sapien
LSLEGDHSTPPSAYGSVK

SEQ ID NO: 248 - protein, 12 aa, homo sapien
SYSPYDMLESIR

SEQ ID NO: 249 - protein, 15 aa, homo sapien
EQHLYYQDQLLPVSR

SEQ ID NO: 250 - protein, 12 aa, homo sapien
YHLGAYTGDDVR

SEQ ID NO: 251 - protein, 17 aa, homo sapien
KEDLVFIFWAPESAPLK

SEQ ID NO: 252 - protein, 11 aa, homo sapien
YALYDATYETK

SEQ ID NO: 253 - protein, 13 aa, homo sapien
DQGTYEDYVEGLR

SEQ ID NO: 254 - protein, 8 aa, homo sapien
EAFQLFDR

SEQ ID NO: 255 - protein, 12 aa, homo sapien
EGNGTVMGAEIR

SEQ ID NO: 256 - protein, 13 aa, homo sapien
ILDSVGIEADDDR

SEQ ID NO: 257 - protein, 14 aa, homo sapien
AQAELVGTADEATR

SEQ ID NO: 258 - protein, 15 aa, homo sapien
NQVALNPQNTVFDAK

SEQ ID NO: 259 - protein, 7 aa, homo sapien
GFDEYMK

SEQ ID NO: 260 - protein, 16 aa, homo sapien
VLGAFSDGLAHLDNLK

SEQ ID NO: 261 - protein, 13 aa, homo sapien
GTFATLSELHCDK

SEQ ID NO: 262 - protein, 13 aa, homo sapien
VNVDAVGGEALGR

SEQ ID NO: 263 - protein, 14 aa, homo sapien
DSLLQDGEFSMDLR

SEQ ID NO: 264 - protein, 14 aa, homo sapien
SSFYVNGLTLGGQK

SEQ ID NO: 265 - protein, 14 aa, homo sapien
STGGAPTFNVTVTK

SEQ ID NO: 266 - protein, 12 aa, homo sapien
ALEESNYELEGK

SEQ ID NO: 267 - protein, 13 aa, homo sapien
QSLEASLAETEGR

SEQ ID NO: 268 - protein, 11 aa, homo sapien
TAAENDFVTLK

SEQ ID NO: 269 - protein, 10 aa, homo sapien
GASGIQGLAR

SEQ ID NO: 270 - protein, 13 aa, homo sapien
NGSGTLDLEEFLR

SEQ ID NO: 271 - protein, 8 aa, homo sapien
SLDADEFR

SEQ ID NO: 272 - protein, 14 aa, homo sapien
QMEQISQFLQAAER

SEQ ID NO: 273 - protein, 12 aa, homo sapien
TLMNLGGLAVAR

SEQ ID NO: 274 - protein, 14 aa, homo sapien
LISWYDNEFGYSNR

SEQ ID NO: 275 - protein, 8 aa, homo sapien
VHLTPEEK

SEQ ID NO: 276 - protein, 20 aa, homo sapien
GNDISSGTVLSDYVGSGPPK

SEQ ID NO: 277 - protein, 14 aa, homo sapien
LYTLVLTDPDAPSR

SEQ ID NO: 278 - protein, 14 aa, homo sapien
SLYASSPGGVYATR

SEQ ID NO: 279 - protein, 17 aa, homo sapien
AIGGGLSSVGGGSSTIK

SEQ ID NO: 280 - protein, 9 aa, homo sapien
SLYGLGGSK

SEQ ID NO: 281 - protein, 13 aa, homo sapien
EVATNSELVQSGK

SEQ ID NO: 282 - protein, 11 aa, homo sapien
LAAIAESGVER

SEQ ID NO: 283 - protein, 9 aa, homo sapien
LTMQNLNDR

SEQ ID NO: 284 - protein, 12 aa, homo sapien
NVDEVGGEALGR

SEQ ID NO: 285 - protein, 10 aa, homo sapien
LEGLEDALQK

SEQ ID NO: 286 - protein, 14 aa, homo sapien
EIEQEAAVELSQLR

SEQ ID NO: 287 - protein, 8 aa, homo sapien
VPDFSEYR

SEQ ID NO: 288 - protein, 11 aa, homo sapien
CEMEQQNQEYK

SEQ ID NO: 289 - protein, 8 aa, homo sapien
FSVNLDVK

SEQ ID NO: 290 - protein, 8 aa, homo sapien
HFSPEELK

SEQ ID NO: 291 - protein, 11 aa, homo sapien
RPFFPFHSPSR

SEQ ID NO: 292 - protein, 11 aa, homo sapien
EGMNIVEAMER

SEQ ID NO: 293 - protein, 9 aa, homo sapien
FEDENFILK

SEQ ID NO: 294 - protein, 9 aa, homo sapien
VSFELFADK

SEQ ID NO: 295 - protein, 12 aa, homo sapien
TINEVENQILTR

SEQ ID NO: 296 - protein, 12 aa, homo sapien
EQADFAIEALAK

SEQ ID NO: 297 - protein, 14 aa, homo sapien
IMGIPEEEQMGLLR

SEQ ID NO: 298 - protein, 14 aa, homo sapien
VSHLLGINVTDFTR

SEQ ID NO: 299 - protein, 8 aa, homo sapien
YYTPTISR

SEQ ID NO: 300 - protein, 14 aa, homo sapien
GDGPVQGIINFEQK

SEQ ID NO: 301 - protein, 12 aa, homo sapien
HVGDLGNVTADK

SEQ ID NO: 302 - protein, 13 aa, homo sapien
ASGQAFELILSPR

SEQ ID NO: 303 - protein, 8 aa, homo sapien
FITIFGTR

SEQ ID NO: 304 - protein, 9 aa, homo sapien
SEIDLFNIR

SEQ ID NO: 305 - protein, 15 aa, homo sapien
YMTISGFQIEETIDR

SEQ ID NO: 306 - protein, 15 aa, homo sapien
TVAGQDAVIVLLGTR

SEQ ID NO: 307 - protein, 11 aa, homo sapien
QITLNDLPVGR

SEQ ID NO: 308 - protein, 13 aa, homo sapien
WIDETPPVDQPSR

(19) United States
(12) Patent Application Publication
(10) Pub. No.: US 2003/0064419 A1
(43) Pub. Date: Apr. 3, 2003

(54) Proteins, genes and their use for diagnosis and treatment of breast cancer

(76) Inventors: Herath Mudiyanselage Athula Chandrasiri Herath, Abingdon (GB); Michael John O'Hare, London (GB); Martin John Page, Abingdon (GB); Rajesh Bhikhu Parekh, Nr. Wendlebury (GB); Michael Derek Waterfield, London (GB)

Correspondence Address: KLAUBER & JACKSON, 411 HACKENSACK AVENUE, HACKENSACK, NJ, 07601

(21) Appl. No.: 09/988,493

(22) Filed: Nov. 20, 2001

Related U.S. Application Data
(63) Continuation of application No. PCT/GB01/01219, filed on Mar. 20, 2001.

(30) Foreign Application Priority Data
Mar. 20, 2000 (GB) 0006695.1
Mar. 24, 2000 (GB) 0007265.2

(51) Int. Cl. G01N 33/574; G01N 33/557

(52) U.S. Cl. 435/7.23; 436/517

(57) ABSTRACT

The present invention relates to the identification of proteins and protein isoforms that are associated with breast cancer and its onset and development, and of genes encoding the same, and to their use for e.g., clinical screening, diagnosis, prognosis, therapy and prophylaxis, as well as for drug screening and development of pharmaceutical products.

### INTRODUCTION

The present invention relates to the identification of proteins and protein isoforms that are associated with breast cancer and its onset and development, and of genes encoding the same, and to their use for e.g., clinical screening, diagnosis, prognosis, therapy and prophylaxis, as well as for drug screening and development of pharmaceutical products.

### BACKGROUND OF THE INVENTION

Breast cancer is the most frequently diagnosed non-skin cancer among women in the United States. It is second only to lung cancer in cancer-related deaths. Approximately 180,000 new cases of breast cancer will be diagnosed in 1997, and about 44,000 women are expected to die from the disease (National Cancer Institute, http://www.nci.nih.org, USA, 1999). In the UK, breast cancer is by far the commonest cancer in women, with 34,600 new cases in 1998 (Cancer Research Campaign, http://www.crc.org.uk, UK, 2000). Ninety-nine percent of breast cancers occur in women. The risk of developing breast cancer steadily increases with age; the lifetime risk of developing breast cancer is estimated to be 1 in 8 for women in the US. The annual cost of breast cancer treatment in the United States is approximately $10 billion (Fuqua, et. al. 2000, American Association for Cancer Research, www.aacr.org, USA). Breast cancer incidence has been rising over the past five decades, but recently it has slowed. This may reflect a period of earlier detection of breast cancers by mammography. A number of established factors can increase a woman's risk of having the disease. These include older age, history of prior breast cancer, significant radiation exposure, strong family history of breast cancer, upper socioeconomic class, nulliparity, early menarche, late menopause, or age at first pregnancy greater than 30 years. Prolonged use of oral contraceptives earlier in life appears to increase risk slightly. Prolonged postmenopausal estrogen replacement increases the risk 20 to 40%. It has been speculated that a decrease in the age at menarche, changing birth patterns, or a rise in the use of exogenous estrogens has contributed to the increase in breast cancer incidence (Fuqua, et. al. 2000, American Association for Cancer Research, www.aacr.org, USA).

Causes of Breast Cancer. Breast cancer is a heterogeneous disease. Although female hormones play a significant role in driving the origin and evolution of many breast tumors, there are a number of other recognized and unknown factors involved. Perturbations in oncogenes identified include amplification of the HER-2 and the epidermal growth factor receptor genes, and overexpression of cyclin D1. Overexpression of these oncogenes has been associated with a significantly poorer prognosis. Similarly, genetic alterations or the loss of tumor suppressor genes, such as the p53 gene, have been well documented in breast cancer and are also associated with a poorer prognosis. Researchers have identified two genes, called BRCA1 and BRCA2, which are predictive of premenopausal familial breast cancer. Genetic risk assessment is now possible, which may enhance the identification of candidates for chemoprevention trials (Fuqua, et. al. 2000, American Association for Cancer Research, www.aacr.org, USA).

Diagnosis. Early diagnosis of breast cancer is vital to secure the most favorable outcome for treatment. Many countries with advanced healthcare systems have instituted screening programs for breast cancer. This typically takes the form of regular x-ray of the breast (mammography) during the 50-60 year old age interval where greatest benefit for this intervention has been shown. Some authorities have advocated the extension of such programs beyond 60 and to the 40-49 age group. Health authorities in many countries have also promoted the importance of regular breast self-examination by women. Abnormalities detected during these screening procedures and cases presenting as symptomatic would typically be confirmed by aspiration cytology, core needle biopsy with a stereotactic or ultrasound technique for nonpalpable lesions, or incisional or excisional biopsy. At the same time other information relevant to treatment options and prognosis, such as oestrogen (ER) and progesterone receptor (PR) status would typically be determined (National Cancer Institute, USA, 2000, Breast Cancer PDQ, www.nci.org).

Disease Staging and Prognosis. Staging of breast cancer is the key to choosing the optimum treatment for each patient and to select those patients who will fare well with less intensive forms of therapy from those for whom intensive therapy is essential. Currently the process of staging involves lump and axillary lymph node biopsies, combined with extensive histopathology. Patients can be incorrectly staged with consequent over- or under-treatment. There is a need for new markers that can be correlated with disease stage and used to reliably guide treatment decisions. Such new markers would not only benefit patients and health care providers by selecting the optimum treatment, but could provide significant cost and time benefits in the histology lab.

Most important is the differentiation of ductal carcinoma in situ (DCIS), a non-invasive tumour of ductal origin, from invasive lobular carcinoma (ILC) and invasive ductal carcinoma (IDC) that have spread into surrounding breast tissue and are more likely to have invaded local lymph nodes. In turn locally invasive disease needs to be distinguished from more widely metastasised disease that requires aggressive chemotherapy.

Those patients with DCIS have been shown to have an excellent prognosis with breast conserving surgery, low dose (50Gy) breast irradiation and Tamoxifen. Patients with invasive lobular or ductal carcinomas need biopsy examination and possible removal of local lymph nodes. These patients show a benefit from radical mastectomy and local lymph node irradiation and may need chemotherapy, with or without hormone therapy. Metastatic breast disease is likely to require aggressive chemotherapy as well as surgery and irradiation. Newer chemotherapeutic agents such as the Taxanes and the immumo therapeutic anti-erbB2 drug Herceptin are likely to be reserved for such patients. There is however a considerable deficit of new drug targets for treating breast cancer. Additionally, many of the proteins that can be used to distinguish between the various categories of breast cancer described above (DCIS, ILC, IDC and metastasised disease), also have the potential to provide new targets for therapy as well.

Treatment. Surgery, radiation therapy, hormone therapy, and chemotherapy are the most common treatments for breast cancer.

Surgery. Most women with breast cancer will have some type of surgery. The purpose of surgery is to remove as much of the cancer as possible. This may be in the form of lumpectomy or more radical mastectomy with breast reconstruction. Surgery may also be combined with other treatments like chemotherapy, hormone therapy, or radiation therapy. Surgery may also be performed to find out whether breast cancer has spread to the lymph nodes under the arm (axillary dissection), to restore a more normal appearance (reconstructive surgery), or to relieve symptoms of advanced cancer.

Chemotherapy. Chemotherapy is the use of anticancer drugs to kill cancer cells. When chemotherapy is given after surgery (adjuvant therapy) it can reduce the chance of cancer recurrence. Chemotherapy can also be used as the main treatment for women whose cancer is widespread when it is found, or spreads widely after initial treatment. Neoadjuvant chemotherapy is typically given before surgery, often to shrink the tumour and make it easier to remove. Chemotherapy is given in cycles, with each period of treatment followed by a recovery period. The total course lasts three to six months. It is often more effective to use several drugs, rather than a single drug alone. The most commonly used combinations are: cyclophosphamide, methotrexate, and fluorouracil (CMF); cyclophosphamide, doxorubicin (Adriamycin), and fluorouracil (CAF); doxorubicin (Adriamycin) and cyclophosphamide (AC), with or without paclitaxel (Taxol); doxorubicin (Adriamycin), followed by CMF.

Radiation therapy. Radiation therapy is commonly applied in breast cancer treatment. It may be used to reduce the size of a tumor before surgery or to destroy cancer cells remaining in the breast, chest wall, or underarm area after surgery.

Hormone therapy and Chemoprevention. The hormone oestrogen can increase the growth of breast cancer cells in some women. A drug such as tamoxifen, which blocks the effect of estrogen, is typically given to counter this growth. Another newer drug, raloxifene, also blocks the effect of oestrogen on breast tissue and breast cancer. There is increasing evidence that these anti-oestrogen treatments may also have a role in chemoprevention of breast cancer in high-risk individuals.

Immunotherapy. Trastuzumab (Herceptin) is a new immunotherapeutic agent that interacts with a growth factor receptor known as c-erbB2/HER2/neu, which is present in small amounts on the surface of normal breast cells and at much higher levels in some breast cancers. This growth factor receptor can cause the cancer to grow and spread faster. Herceptin can inhibit the c-erbB2/HER2/neu protein from promoting breast cancer cell growth. It may also help the immune system to better attack the cancer. Herceptin is currently typically administered after standard hormonal or chemotherapy no longer appears to be working (American Cancer Society, 2000, USA, www.cancer.org).

Therapeutic Challenges. The major challenges in breast cancer treatment are to improve early detection rates, to find new non-invasive markers that can be used to follow disease progression and identify relapse, and to find improved and less toxic therapies, especially for more advanced disease where 5 year survival is still very poor. There is a great need to identify targets which are more specific to cancer cells, ideally ones which are expressed on the surface of the tumor cells so that they can be attacked by promising new approaches like immunotherapy and targeted toxins.

Therefore, a need exists to identify breast cancer associated proteins for use as sensitive and specific biomarkers for the diagnosis of breast cancer in living subjects. Additionally, there is a clear need for new therapeutic agents for breast cancer that work quickly, potently, specifically, and with fewer side effects.

### SUMMARY OF THE INVENTION

The present invention provides methods and compositions for clinical screening, diagnosis, prognosis, therapy and prophylaxis of breast cancer, for monitoring the effectiveness of breast cancer treatment, for selecting participants in clinical trials, for identifying subjects most likely to respond to a particular therapeutic treatment and for screening and development of drugs for treatment of breast cancer. A first aspect of the invention provides methods for diagnosis of breast cancer that comprise analyzing a sample of breast tissue by two-dimensional electrophoresis to detect the presence or level of at least one Breast Cancer-Associated Feature (BF), e.g., one or more of the BFs disclosed herein or any suitable combination thereof. These methods are also suitable for clinical screening, prognosis, monitoring the results of therapy, identifying subjects most likely to respond to a particular therapeutic treatment, for drug screening and development, and identification of new targets for drug treatment.

A second aspect of the invention provides methods for diagnosis of breast cancer that comprise detecting in a sample of breast tissue the presence or level of at least one Breast Cancer-Associated Protein Isoform (BPI), e.g., one or more of the BPIs disclosed herein or any combination thereof. These methods are also suitable for clinical screening, prognosis, monitoring the results of therapy, identifying subjects most likely to respond to a particular therapeutic treatment, drug screening and development, and identification of new targets for drug treatment.

A third aspect of the invention provides antibodies, e.g. monoclonal and polyclonal antibodies capable of immunospecific binding to a BPI, e.g., a BPI disclosed herein.

A fourth aspect of the invention provides a preparation comprising an isolated BPI, i.e., a BPI substantially free from proteins or protein isoforms having a significantly different isoelectric point or a significantly different apparent molecular weight from the BPI.

A fifth aspect of the invention provides methods of treating breast cancer, comprising administering to a subject a therapeutically effective amount of an agent that modulates (e.g., upregulates or downregulates) the expression or activity (e.g. enzymatic or binding activity), or both, of a BPI in subjects having breast cancer, in order to prevent or delay the onset or development of breast cancer, to prevent or delay the progression of breast cancer, or to ameliorate the symptoms of breast cancer.

A sixth aspect of the invention provides methods of screening for agents that modulate (e.g., upregulate or downregulate) a characteristic of, e.g., the expression or the enzymatic or binding activity, of a BPI, a BPI analog, or a BPI-related polypeptide.

### DEFINITIONS

As used herein, the term “feature” refers to a spot detected in a 2D gel, and the term “Breast Cancer-Associated Feature” (BF) refers to a feature that is differentially present in a sample (e.g. a sample of breast tissue) from a subject having breast cancer compared with a sample (e.g. a sample of breast tissue) from a subject free from breast cancer. As used herein, a feature (or a protein isoform of BPIs, as defined infra) is “differentially present” in a first sample with respect to a second sample when a method for detecting the feature, isoform or BPI (e.g., 2D electrophoresis or an immunoassay) gives a different signal when applied to the first and second samples. A feature, isoform or BPI is “increased” in the first sample with respect to the second if the method of detection indicates that the feature, isoform or BPI is more abundant in the first sample than in the second sample, or if the feature, isoform or BPI is detectable in the first sample and undetectable in the second sample. Conversely, a feature, isoform or BPI is “decreased” in the first sample with respect to the second if the method of detection indicates that the feature, isoform or BPI is less abundant in the first sample than in the second sample or if the feature, isoform or BPI is undetectable in the first sample and detectable in the second sample.

The term “blood” as used herein includes serum and plasma. Serum refers to the supernatant fluid produced by clotting and centrifugal sedimentation of a blood sample. Plasma refers to the supernatant fluid produced by inhibition of clotting (for example, by citrate or EDTA) and centrifugal sedimentation of a blood sample.

The term “diagnosis” as used herein refers to screening for breast cancer, differential diagnosis of breast cancer versus other cancers, assessing the stage or severity of a particular breast cancer, prognosis, selection of participants in clinical trials, selection of subjects most likely to benefit from a particular therapeutic treatment, monitoring of therapy and response to therapy.

The term “treatment” as used herein refers to prevention, prophylaxis and treatment of subjects with diagnosed breast cancer, or at risk of developing breast cancer.

The term “breast cancer stage” as used herein refers to one of the breast cancer stages among the following: invasive lobular carcinoma (ILC), invasive ductal carcinoma (IDC) and widely metastasised disease (PE).

As used herein, the terms “MW” and “pI” are defined, respectively, to mean the apparent molecular weight in Daltons and the apparent isoelectric point of a feature or protein isoform as measured in exact accordance with the Reference Protocol identified in Section 6 below.

The term “Expression Reference Feature” (“ERF”) as used herein refers to a feature whose abundance is invariant, within the limits of variability of the Preferred Technology, in the population of subjects being examined.

The term “BPI analog” as used herein refers to a polypeptide that possesses a similar or identical function as a BPI but need not necessarily comprise an amino acid sequence that is similar or identical to the amino acid sequence of the BPI, or possess a structure that is similar or identical to that of the BPI. As used herein, an amino acid sequence of a polypeptide is “similar” to that of a BPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the BPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the BPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the BPI. As used herein, a polypeptide with “similar structure” to that of a BPI refers to a polypeptide that has a similar secondary, tertiary or quartemary structure as that of the BPI. The structure of a polypeptide can be determined by methods known to those skilled in the art, including but not limited to, X-ray crystallography, nuclear magnetic resonance, and crystallographic electron microscopy.

The term “BPI fusion protein” as used herein refers to a polypeptide that comprises (i) an amino acid sequence of a BPI, a BPI fragment, a BPI-related polypeptide or a fragment of a BPI-related polypeptide and (ii) an amino acid sequence of a heterologous polypeptide (i.e., a non-BPI, non-BPI fragment or non-BPI-related polypeptide).

The term “BPI homolog” as used herein refers to a polypeptide that comprises an amino acid sequence similar to that of a BPI but does not necessarily possess a similar or identical function as the BPI.

The term “BPI ortholog” as used herein refers to a non-human polypeptide that (i) comprises an amino acid sequence similar to that of a BPI and (ii) possesses a similar or identical function to that of the BPI.

The term “BPI-related polypeptide” as used herein refers to a BPI homolog, a BPI analog, an isoform of BPI, a BPI ortholog, or any combination thereof.

The term “derivative” as used herein refers to a polypeptide that comprises an amino acid sequence of a second polypeptide which has been altered by the introduction of amino acid residue substitutions, deletions or additions. The derivative polypeptide possesses a similar or identical function as the second polypeptide.

The term “fragment” as used herein refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a BPI may or may not possess a functional activity of the a second polypeptide.

The term “fold change” includes “fold increase” and “fold decrease” and refers to the relative increase or decrease in abundance of an BF or the relative increase or decrease in expression or activity of a polypeptide (e.g. a BPI) in a first sample or sample set compared to a second sample (or sample set). An BF or polypeptide fold change may be measured by any technique known to those of skill in the art, however the observed increase or decrease will vary depending upon the technique used. Preferably, fold change is determined herein as described in the Examples infra.

The term “isoform” as used herein refers to variants of a polypeptide that are encoded by the same gene, but that differ in their pI or MW, or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that exists in only a single form, i.e., it is not expressed as several variants.

The term “modulate” when used herein in reference to expression or activity of a BPI or a BPI-related polypeptide refers to the upregulation or downregulation of the expression or activity of the BPI or a BPI-related polypeptide. Based on the present disclosure, such modulation can be determined by assays known to those of skill in the art or described herein.

The term “modulator” as used herein refers to an agent that modulates the expression or activity of a BPI or BPI-related polypeptide, such as, e.g., agonists or antagonists of a BPI or BPI-related polypeptide, and to blockers of protein-protein interactions that induce a modulation of the expression or activity of a BPI or BPI-related polypeptide.

As used herein, “breast tissue” refers to cells derived from breast tissue from the breast itself, as well as the tissue adjacent to and/or within the strata underlying the breast.

The percent identity of two amino acid sequences or of two nucleic acid sequences is generally determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions ×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

### BRIEF DESCRIPTION OF THE FIGURES

FIG. 1 is an image obtained from 2-dimensional electrophoresis of breast tissue, which has been annotated to identify thirteen landmark features, designated BT1, BT2, BT3, BT4, BT5, BT6, BT7, BT8, BT9, BT10, BT11, BT12, and BT13.

FIG. 2 is a Venn diagram showing the 7 clusters resulting from the statistical analysis of the profiles.

### DETAILED DESCRIPTION OF THE INVENTION

The invention described in detail below provides methods and compositions for diagnosis of breast cancer in a subject and for drug screening and development of pharmaceutical products. The invention also encompasses the administration of therapeutic compositions to a subject to treat or prevent breast cancer. The subject may be a non-human mammal, but is preferably human, more preferably a human adult, i.e. a human subject at least 21 (more preferably at least 35, at least 50, at least 60, at least 70, or at least 80) years old. For clarity of disclosure, and not by way of limitation, the invention will be described with respect to the analysis of breast tissue samples. However, as one skilled in the art will appreciate, the assays and techniques described below can be applied to other types of samples (e.g. blood, saliva or urine), including a tissue, a tissue sample from a subject at risk of having or developing breast cancer (e.g. a biopsy such as a lymph node biopsy) or homogenate thereof. The methods and compositions of the present invention are useful for diagnosis of a living subject, but may also be used for postmortem diagnosis in a subject, for example, to identify family members of the subject who are at risk of developing the same disease.

### Breast Cancer-Associated Features (BFs)

In one aspect of the invention, two-dimensional electrophoresis is used to analyze breast tissue from a subject, preferably a living subject, in order to detect or quantify the expression of one or more Breast Cancer-Associated Features (BFs) for prevention or diagnosis of breast cancer, or for development of pharmaceutical products. As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97GB3307 (published as WO 98/23950) and in U.S. Pat. No. 6,064,654, both filed Dec. 1, 1997, each of which is incorporated herein by reference in its entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

Preferably, the relative abundance of a feature in two samples is determined in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF), e.g. the ERFs disclosed below, or (c) more preferably to the total signal detected from all proteins in the sample.

Secondly, the normalized signal for the feature in one sample or sample set is compared with the normalized signal for the same feature in another sample or sample set in order to identify features that are “differentially present” in the first sample (or sample set) with respect to the second.

The BFs disclosed herein have been identified by comparing breast tissue samples from subjects having breast cancer against breast tissue samples from subjects free from breast cancer. Subjects free from breast cancer include subjects with no known disease or condition (normal subjects) and subjects with diseases (including mammary pathologies) other than breast cancer (“Control Subjects”).

TABLE I
BFs Identified in Subjects Having Breast Cancer

| Table I | | | Fold |
|---|---|---|---|
| BF# | pl | MW (Da) | Change * |
| BF-1 | 6.73 | 57363 | − |
| BF-2 | 4.85 | 49013 | − |
| BF-3 | 6.33 | 79913 | − |
| BF-4 | 5.45 | 53541 | − |
| BF-5 | 5.59 | 33775 | − |
| BF-6 | 5.58 | 52905 | − |
| BF-7 | 10.52 | 48758 | − |
| BF-8 | 5.35 | 53897 | − |
| BF-9 | 4.92 | 52046 | − |
| BF-10 | 6.51 | 56205 | − |
| BF-11 | 7.61 | 53837 | − |
| BF-12 | 4.91 | 42632 | − |
| BF-13 | 6.71 | 28131 | − |
| BF-14 | 5.39 | 44908 | − |
| BF-15 | 5.49 | 26295 | − |
| BF-16 | 5.41 | 51333 | − |
| BF-17 | 6.54 | 54317 | − |
| BF-19 | 5.41 | 54116 | − |
| BF-20 | 6.84 | 55209 | − |
| BF-21 | 6.40 | 55957 | − |
| BF-22 | 7.55 | 21761 | − |
| BF-23 | 5.28 | 54631 | − |
| BF-24 | 4.95 | 42165 | − |
| BF-25 | 4.63 | 30260 | − |
| BF-26 | 5.99 | 57554 | − |
| BF-27 | 5.73 | 52283 | − |
| BF-31 | 7.23 | 56167 | − |
| BF-33 | 4.67 | 47474 | + |
| BF-34 | 7.12 | 32939 | + |
| BF-35 | 6.54 | 34373 | + |
| BF-36 | 5.50 | 22620 | + |
| BF-37 | 4.99 | 30472 | + |
| BF-38 | 5.16 | 68895 | + |
| BF-39 | 4.77 | 33587 | + |
| BF-40 | 6.08 | 12362 | + |
| BF-41 | 5.46 | 34675 | + |
| BF-42 | 4.91 | 40702 | + |
| BF-43 | 5.07 | 70174 | + |
| BF-44 | 4.99 | 34791 | + |
| BF-45 | 4.84 | 13610 | + |
| BF-46 | 5.34 | 29967 | + |
| BF-47 | 4.60 | 37942 | + |
| BF-48 | 6.97 | 11833 | + |
| BF-49 | 6.22 | 12131 | + |
| BF-50 | 5.47 | 23683 | + |
| BF-51 | 5.68 | 70074 | + |
| BF-52 | 5.89 | 69864 | + |
| BF-53 | 4.68 | 41976 | + |
| BF-54 | 6.33 | 31022 | + |
| BF-55 | 5.96 | 26272 | + |
| BF-56 | 5.98 | 38417 | + |
| BF-57 | 4.68 | 14062 | + |
| BF-60 | 4.50 | 17973 | + |
| BF-62 | 4.54 | 44349 | + |
| BF-64 | 4.91 | 21294 | + |
| BF-65 | 4.60 | 54421 | + |
| BF-67 | 5.40 | 28445 | + |
| BF-71 | 5.39 | 49797 | + |
| BF-72 | 6.75 | 24466 | + |
| BF-73 | 6.65 | 36425 | − |
| BF-75 | 8.13 | 9988 | − |
| BF-76 | 5.04 | 28469 | + |
| BF-78 | 10.13 | 21133 | + |
| BF-80 | 7.86 | 82682 | + |
| BF-81 | 7.29 | 33646 | + |
| BF-83 | 6.55 | 58102 | + |
| BF-84 | 5.84 | 50081 | + |
| BF-85 | 6.32 | 28158 | − |
| BF-86 | 5.39 | 29939 | − |
| BF-89 | 5.4 | 12234 | + |
| BF-90 | 6.70 | 32475 | + |
| BF-92 | 5.97 | 49895 | + |
| BF-94 | 5.75 | 70640 | + |
| BF-99 | 6.19 | 41490 | + |
| BF-101 | 5.78 | 68948 | + |
| BF-102 | 8.94 | 32089 | + |
| BF-103 | 7.83 | 38361 | + |
| BF-106 | 8.00 | 23764 | + |
| BF-112 | 7.72 | 36705 | + |
| BF-114 | 4.67 | 16800 | − |
| BF-122 | 5.25 | 32285 | + |
| BF-123 | 5.89 | 34642 | + |
| BF-125 | 4.78 | 35147 | + |
| BF-126 | 7.54 | 13341 | + |
| BF-127 | 4.69 | 37235 | + |
| BF-131 | 5.67 | 39910 | + |
| BF-134 | 5.55 | 32551 | + |
| BF-135 | 6.38 | 25179 | + |
| BF-137 | 6.27 | 32684 | + |
| BF-138 | 8.02 | 10932 | + |
| BF-201 | 6.1 | 117340 | + |
| BF-202 | 5.94 | 97668 | + |
| BF-203 | 5.66 | 66584 | − |
| BF-204 | 6.33 | 58624 | − |
| BF-205 | 9.6 | 54437 | − |
| BF-206 | 6.04 | 53124 | + |
| BF-207 | 5.37 | 50199 | − |
| BF-208 | 6.66 | 47564 | − |
| BF-209 | 5.99 | 45443 | + |
| BF-210 | 8.39 | 43869 | + |
| BF-211 | 6.08 | 40612 | + |
| BF-212 | 4.69 | 38828 | + |
| BF-213 | 8.06 | 39259 | + |
| BF-214 | 5.77 | 36187 | + |
| BF-215 | 5.99 | 36060 | − |
| BF-216 | 6.26 | 32119 | + |
| BF-217 | 5.44 | 30182 | − |
| BF-218 | 5.21 | 29877 | − |
| BF-219 | 5.24 | 26524 | + |
| BF-220 | 4.82 | 21630 | + |
| BF-221 | 6.83 | 21393 | + |
| BF-222 | 8.52 | 21327 | + |
| BF-223 | 7.09 | 20001 | + |
| BF-224 | 4.65 | 18637 | − |
| BF-225 | 7.63 | 17346 | − |
| BF-226 | 7.89 | 12605 | + |
| BF-227 | 5.35 | 11395 | + |
| BF-228 | 5.7 | 30474 | + |
| BF-229 | 6.1 | 28798 | + |
| BF-230 | 6.08 | 49018 | + |
| BF-231 | 5.85 | 28892 | + |
| BF-232 | 9.66 | 30698 | + |
| BF-233 | 8.07 | 101343 | − |
| BF-234 | 5.77 | 96415 | + |
| BF-235 | 6.92 | 56452 | − |
| BF-236 | 7.87 | 55046 | − |
| BF-237 | 7.77 | 54027 | − |
| BF-238 | 6.46 | 53651 | − |
| BF-239 | 7.52 | 48780 | − |
| BF-240 | 5.73 | 39758 | + |
| BF-241 | 6.63 | 21542 | − |
| BF-242 | 7.44 | 19090 | + |
| BF-243 | 6.95 | 18163 | + |
| BF-244 | 5.00 | 40576 | + |
| BF-245 | 8.41 | 27823 | + |
| BF-246 | 4.61 | 17035 | − |
| BF-247 | 6.84 | 13088 | + |
| BF-248 | 4.97 | 15909 | + |
| BF-249 | 5.05 | 150793 | + |
| BF-250 | 7.77 | 118551 | + |
| BF-251 | 7.58 | 112993 | − |
| BF-252 | 6.65 | 109903 | + |
| BF-253 | 10.58 | 107058 | − |
| BF-254 | 6.29 | 100291 | − |
| BF-255 | 5.61 | 79733 | + |
| BF-256 | 6.97 | 78400 | + |
| BF-257 | 5.12 | 69570 | + |
| BF-258 | 5.60 | 62015 | − |
| BF-259 | 7.40 | 58529 | + |
| BF-260 | 8.59 | 58137 | − |
| BF-261 | 5.63 | 57668 | − |
| BF-262 | 8.31 | 56802 | − |
| BF-263 | 8.19 | 56641 | − |
| BF-264 | 8.44 | 56704 | − |
| BF-265 | 7.57 | 56133 | − |
| BF-266 | 6.09 | 54734 | − |
| BF-267 | 8.21 | 54623 | − |
| BF-268 | 8.39 | 54326 | − |
| BF-269 | 5.50 | 53757 | − |
| BF-270 | 4.95 | 53870 | − |
| BF-271 | 4.78 | 50377 | − |
| BF-272 | 7.89 | 49724 | − |
| BF-273 | 5.68 | 47695 | + |
| BF-274 | 9.28 | 46617 | + |
| BF-275 | 9.90 | 44863 | + |
| BF-276 | 9.47 | 44888 | + |
| BF-277 | 6.49 | 43639 | + |
| BF-278 | 5.01 | 42154 | + |
| BF-279 | 10.31 | 37137 | − |
| BF-280 | 6.65 | 35639 | − |
| BF-281 | 8.05 | 35007 | − |
| BF-282 | 5.97 | 35101 | + |
| BF-283 | 4.51 | 33998 | − |
| BF-284 | 5.24 | 33933 | + |
| BF-285 | 7.51 | 32133 | + |
| BF-286 | 6.46 | 31977 | + |
| BF-287 | 6.35 | 31866 | + |
| BF-288 | 5.03 | 30887 | + |
| BF-289 | 6.54 | 29745 | + |
| BF-290 | 4.97 | 29618 | − |
| BF-291 | 11.61 | 28279 | − |
| BF-292 | 5.54 | 23266 | − |
| BF-293 | 5.40 | 22590 | + |
| BF-294 | 9.43 | 21085 | − |
| BF-295 | 4.71 | 19697 | − |
| BF-296 | 4.74 | 18536 | + |
| BF-297 | 5.97 | 17794 | − |
| BF-298 | 4.60 | 16974 | + |
| BF-299 | 6.36 | 15927 | + |
| BF-300 | 6.56 | 15911 | + |
| BF-301 | 9.67 | 13895 | + |
| BF-302 | 4.57 | 13644 | + |
| BF-303 | 6.49 | 13149 | + |
| BF-304 | 7.09 | 12765 | + |
| BF-305 | 5.07 | 11927 | + |
| BF-306 | 6.39 | 11989 | + |
| BF-307 | 7.57 | 11987 | + |
| BF-308 | 8.13 | 11900 | + |
| BF-309 | 4.91 | 11597 | + |
| BF-310 | 9.64 | 11633 | + |
| BF-311 | 4.59 | 11454 | + |
| BF-312 | 8.12 | 11393 | + |
| BF-313 | 8.85 | 11298 | + |
| BF-314 | 9.70 | 11115 | + |
| BF-315 | 4.56 | 10783 | + |
| BF-316 | 4.86 | 10597 | + |
| BF-317 | 6.45 | 10483 | + |
| BF-318 | 5.84 | 57392 | − |
| BF-319 | 4.83 | 52210 | − |
| BF-320 | 7.89 | 42419 | + |
| BF-321 | 6.71 | 42419 | − |
| BF-322 | 8.08 | 41976 | + |
| BF-323 | 9.13 | 34376 | + |
| BF-324 | 4.85 | 32089 | + |
| BF-325 | 4.50 | 32089 | + |
| BF-326 | 6.67 | 29298 | + |
| BF-327 | 5.46 | 28516 | + |
| BF-328 | 5.75 | 22611 | + |
| BF-329 | 4.65 | 21268 | + |
| BF-330 | 5.46 | 20005 | + |
| BF-331 | 7.94 | 17904 | + |
| BF-332 | 7.17 | 13562 | + |
| BF-333 | 4.81 | 11957 | + |
| BF-334 | 5.52 | 10749 | + |
| BF-335 | 6.09 | 38455 | + |
| BF-336 | 7.79 | 36150 | + |
| BF-337 | 5.07 | 32640 | + |
| BF-338 | 8.05 | 19328 | + |
| BF-339 | 8.17 | 17431 | + |
| BF-340 | 6.55 | 16585 | + |
| BF-341 | 5.92 | 12692 | + |
| BF-342 | 4.98 | 58155 | + |
| BF-343 | 9.25 | 29936 | + |
| BF-344 | 5.96 | 29803 | + |
| BF-345 | 5.18 | 9650 | + |
| BF-346 | 6.43 | 31755 | + |
| BF-347 | 7.68 | 28267 | + |
| BF-348 | 6.65 | 11445 | + |
| BF-349 | 7.96 | 54540 | − |
| BF-350 | 4.65 | 23162 | − |
| BF-351 | 4.77 | 40050 | + |
| BF-352 | 6.29 | 36762 | − |
| BF-353 | 7.41 | 55021 | − |
| BF-354 | 8.65 | 54440 | − |
| BF-355 | 4.80 | 23721 | − |
| BF-356 | 4.85 | 17566 | − |
| BF-357 | 7.36 | 11451 | + |
| BF-358 | 4.83 | 41551 | + |
| BF-359 | 6.18 | 57756 | − |
| BF-360 | 6.24 | 57140 | − |
| BF-361 | 7.44 | 56631 | − |
| BF-362 | 7.93 | 56475 | − |
| BF-363 | 10.19 | 55984 | − |
| BF-364 | 6.16 | 53276 | − |
| BF-365 | 5.85 | 52603 | − |
| BF-366 | 4.95 | 50611 | − |
| BF-367 | 5.63 | 50779 | − |
| BF-368 | 5.10 | 30895 | + |
| BF-369 | 5.34 | 28080 | + |
| BF-370 | 6.84 | 25632 | + |
| BF-371 | 4.70 | 23016 | − |
| BF-372 | 7.52 | 20516 | − |
| BF-373 | 7.16 | 12109 | + |
| BF-374 | 7.64 | 54567 | − |
| BF-375 | 5.58 | 11246 | + |
| BF-376 | 5.92 | 15600 | − |
| BF-377 | 5.74 | 11237 | + |
| BF-378 | 6.14 | 127960 | − |
| BF-379 | 5.24 | 117050 | − |
| BF-380 | 6.03 | 83606 | − |
| BF-381 | 8.07 | 74827 | − |
| BF-382 | 8.43 | 74810 | − |
| BF-383 | 6.24 | 72679 | − |
| BF-384 | 6.54 | 71457 | − |
| BF-385 | 10.39 | 63571 | − |
| BF-386 | 6.35 | 62181 | − |
| BF-387 | 5.02 | 58032 | − |
| BF-388 | 5.66 | 57297 | − |
| BF-389 | 6.34 | 55702 | − |
| BF-390 | 5.65 | 53588 | − |
| BF-391 | 6.60 | 53462 | − |
| BF-392 | 6.08 | 52448 | − |
| BF-393 | 5.17 | 49168 | − |
| BF-394 | 5.85 | 48991 | + |
| BF-395 | 4.74 | 48818 | − |
| BF-396 | 6.89 | 46381 | + |
| BF-397 | 5.45 | 40176 | + |
| BF-398 | 4.68 | 37020 | − |
| BF-399 | 8.43 | 36084 | − |
| BF-400 | 5.70 | 32253 | − |
| BF-401 | 8.29 | 32300 | + |
| BF-402 | 4.53 | 25717 | + |
| BF-403 | 9.31 | 23662 | + |
| BF-404 | 7.25 | 22880 | + |
| BF-405 | 4.68 | 17543 | − |
| BF-406 | 5.55 | 17447 | + |
| BF-407 | 4.63 | 17126 | − |
| BF-408 | 5.69 | 15919 | + |
| BF-409 | 4.95 | 15725 | + |
| BF-410 | 4.60 | 15224 | + |
| BF-411 | 5.09 | 14959 | + |
| BF-412 | 7.00 | 14079 | − |
| BF-413 | 8.57 | 10730 | − |
| BF-414 | 8.75 | 23225 | + |
| BF-415 | 5.79 | 53955 | − |
| BF-416 | 5.10 | 38537 | + |
| BF-417 | 5.02 | 31271 | + |
| BF-418 | 10.27 | 28882 | − |
| BF-419 | 9.42 | 21438 | + |
| BF-420 | 10.22 | 24662 | − |
| BF-421 | 9.49 | 72204 | − |

*“−“ indicates a feature that is decreased in the breast tissue of subjects having breast cancer as compared with the breast tissue of subjects free from breast cancer.

For any given BF, the signal obtained upon analyzing breast tissue from subjects having breast cancer relative to the signal obtained upon analyzing breast tissue from subjects free from breast cancer will depend upon the particular analytical protocol and detection technique that is used. Accordingly, the present invention contemplates that each laboratory will, based on the present description, establish a reference range for each BF in subjects free from breast cancer according to the analytical protocol and detection technique in use, as is conventional in the diagnostic art. Preferably, at least one positive control breast tissue sample from a subject known to have breast cancer or at least one negative control breast tissue sample from a subject known to be free from breast cancer (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no discemable protein feature.

In a preferred embodiment, the signal associated with an BF in the breast tissue of a subject (e.g., a subject suspected of having or known to have breast cancer) is normalized with reference to one or more ERFs detected in the same 2D gel. As will be apparent to one of ordinary skill in the art, such ERFs may readily be determined by comparing different samples using the Preferred Technology. Suitable ERFs include (but are not limited to) that described in the following Tables.

TABLE II
ERFs in Breast Tissue of Subjects Having
Invasive Ductal Carcinoma

| Table II | | |
|---|---|---|
| ERF# | pI | MW |
| ERF-1 | 4.94 | 39358 |
| ERF-2 | 5.63 | 28502 |

TABLE III
ERFs in Pleural Effusion Cells of Subjects
Having Metastasized Breast Cancer

| Table III | | |
|---|---|---|
| ERF# | pI | MW |
| ERF-3 | 6.03 | 39797 |
| ERF-4 | 5.07 | 37690 |

TABLE IV
ERFs in Breast Tissue of Subjects
Having Invasive Lobular Carcinoma

| Table IV | | |
|---|---|---|
| ERF# | pI | MW |
| ERF-5 | 7.29 | 33646 |
| ERF-6 | 6.63 | 28806 |

TABLE V
ERFs in Breast Tissue of Control Subjects

| Table V | | |
|---|---|---|
| ERF# | pI | MW |
| ERF-7 | 8.05 | 35007 |
| ERF-8 | 6.97 | 11833 |

As those of skill in the art will readily appreciate, the measured MW and pI of a given feature or protein isoform will vary to some extent depending on the precise protocol used for each step of the 2D electrophoresis and for landmark matching. When the Reference Protocol is followed and when samples are run in duplicate or a higher number of replicates, variation in the measured mean pI of a BF or BPI is typically less than 3% and variation in the measured mean MW of a BF or BPI is typically less than 5%. Where the skilled artisan wishes to deviate from the Reference Protocol, calibration experiments should be performed to compare the MW and pI for each BF or protein isoform as detected (a) by the Reference Protocol and (b) by the deviant protocol.

As will be apparent from the statistical analysis described in Section 6 below, the BFs listed in Table I above can be used for diagnosis or for development of pharmaceutical products.

In one embodiment of the invention, breast tissue from a subject (e.g., a subject suspected of having breast cancer) is analyzed by 2D electrophoresis for quantitative detection of one or more of the following BFs: BF-1, BF-2, BF-3, BF-4, BF-5, BF-6, BF-7, BF-8, BF-9, BF-10, BF-11, BF-12, BF-13, BF-14, BF-15, BF-16, BF-17, BF-19, BF-20, BF-21, BF-22, 4 BF-23, BF-24, BF-26, BF-73, BF-114, BF-233, BF-235, BF-236, BF-237, BF-238, BF-239, BF-241, BF-246, BF-251, BF-253, BF-254, BF-258, BF-260, BF-261, BF-263, BF-264, BF-265, BF-266, BF-267, BF-268, BF-269, BF-270, BF-271, BF-272, BF-279, BF-280, BF-281, BF-283, BF-290, BF-291, BF-292, BF-294, BF-295, BF-297, BF-319, BF-321, BF-349, BF-350, BF-353, BF-354, BF-355, BF-356, BF-359, BF-360, BF-361, BF-362, BF-363, BF-364, BF-365, BF-366, BF-367, BF-371, BF-372, BF-374, BF-376. A decreased abundance of said one or more BFs in the breast tissue from the subject relative to breast tissue from a subject or subjects free from breast cancer (e.g., a control sample or a previously determined reference range) indicates the presence of invasive ductal carcinoma.

In another embodiment of the invention, breast tissue from a subject is analyzed by 2D electrophoresis for quantitative detection of one or more of the following BFs: BF-33, BF-34, BF-35, BF-36, BF-37, BF-38, BF-39, BF-40, BF-41, BF-42, BF-43, BF-44, BF-46, BF-47, BF-48, BF-49, BF-50, BF-51, BF-52, BF-53, BF-54, BF-55, BF-57, BF-62, BF-64, BF-65, BF-67, BF-71, BF-72, BF-78, BF-90, BF-99, BF-101, BF-102, BF-106, BF-122, BF-123, BF-125, BF-126, BF-127, BF-131, BF-134, BF-138, BF-234, BF-240, BF-242, BF-243, BF-244, BF-245, BF-247, BF-248, BF-249, BF-250, BF-252, BF-256, BF-257, BF-259, BF-273, BF-274, BF-275, BF-276, BF-277, BF-278, BF-282, BF-284, BF-285, BF-286, BF-287, BF-288, BF-289, BF-293, BF-296, BF-298, BF-299, BF-300, BF-301, BF-302, BF-303, BF-304, BF-305, BF-306, BF-307, BF-308, BF-309, BF-310, BF-311, BF-312, BF-313, BF-314, BF-315, BF-316, BF-317, BF-320, BF-322, BF-323, BF-325, BF-326, BF-327, BF-328, BF-329, BF-330, BF-331, BF-332, BF-333, BF-334, BF-335, BF-336, BF-337, BF-338, BF-339, BF-340, BF-341, BF-342, BF-343, BF-344, BF-345, BF-346, BF-347, BF-348, BF-357, BF-358, BF-368, BF-369, BF-370, BF-373, BF-375, BF-377. An increased abundance of said one or more BFs in the breast tissue from the subject relative to breast tissue from a subject or subjects free from breast cancer (e.g., a control sample or a previously determined reference range) indicates the presence of invasive ductal carcinoma.

In yet another embodiment, breast tissue from a subject is analyzed by 2D electrophoresis for quantitative detection of (a) one or more BFs or any combination of them, whose decreased abundance indicates the presence of invasive ductal carcinoma, i.e., BF-1, BF-2, BF-3, BF-4, BF-5, BF-6, BF-7, BF-8, BF-9, BF-10, BF-1 1, BF-12, BF-15, BF-16, BF-17, BF-19, BF-20, BF-21, BF-22, BF-23, BF-24, BF-26, BF-73, BF-233, BF-235, BF-236, BF-237, BF-238, BF-239, BF-241, BF-246, BF-251, BF-253, BF-254 BF-258, BF-260, BF-261, BF-262, BF-263, BF-264, BF-265, BF-266, BF-267, BF-268, BF-269, BF-270, BF-271, BF-272, BF-279, BF-280, BF-281, BF-283, BF-290, BF-294, BF-295, BF-297, BF-318, BF-319, BF-321, BF-349, BF-350, BF-353, BF-354, BF-355, BF-356, BF-359, BF-360, BF-361, BF-362, BF-363, BF-364, BF-365, BF-366, BF-367, BF-371, BF-372, BF-374, BF-376, and (b) one or more BFs or any combination of them, whose increased abundance indicates the presence of invasive ductal carcinoma i.e., BF-33, BF-34, BF-35, BF-36, BF-37, BF-38, BF-39, BF-40, BF-41, BF-42, BF-43, BF-44, BF-46, BF-47, BF-48, BF-49, BF-50, BF-51, BF-52, BF-53, BF-54, BF-55, BF-57, BF-62, BF-64, BF-65, BF-67, BF-71, BF-72, BF-78, BF-90, BF-99, BF-101, BF-102, BF-106, BF-122, BF-123, BF-125, BF-126, BF-127, BF-131, BF-134, BF-138, BF-234, BF-240. BF-242, BF-243, BF-244, BF-245, BF-247, BF-248, BF-249, BF-250, BF-252, BF-255, BF-256, BF-257, BF-259, BF-273, BF-275, BF-276, BF-277, BF-278, BF-282, BF-285, BF-286, BF-287, BF-288, BF-289, BF-293, BF-296, BF-298, BF-299, BF-300, BF-301, BF-302, BF-303, BF-304, BF-305, BF-306, BF-307, BF-308, BF-309, BF-310, BF-312, BF-313, BF-314, BF-315, BF-316, BF-317, BF-320, BF-322, BF-323, BF-324, BF-326, BF-327, BF-328, BF-329, BF-330, BF-331, BF-332, BF-333, BF-334, BF-335, BF-336, BF-346, BF-347, BF-348, BF-357, BF-358, BF-368, BF-369, BF-370, BF-373, BF-375, BF-377.

In yet another embodiment of the invention, breast tissue from a subject is analyzed by 2D electrophoresis for quantitative detection of one or more of the following BFs: BF-1, BF-2, BF-3, BF-4, BF-5, BF-6, BF-7, BF-8, BF-9, BF-10, BF-11, BF-12, BF-13 BF-14, BF-15, BF-16, BF-17, BF-19, BF-20, BF-21, BF-22, BF-23, BF-24, BF-24, BF-34, BF-35, BF-36, BF-37, BF-38, BF-39, BF-40, BF-41, BF-42, BF-43, BF-44, BF-46, BF-47, BF-48, BF-49, BF-50, BF-51, BF-52, BF-53, BF-54, BF-55, BF-57, BF-62, BF-64, BF-65, BF-67, BF-71, BF-72, BF-73, BF-78, BF-90, BF-99, BF-101, BF-106, BF-114, BF-122, BF-123, BF-125, BF-126, BF-127, BF-131, BF-134, BF-138, BF-233, BF-234, BF-235, BF-236, BF-237, BF-238, BF-239, BF-240, BF-241, BF-242, BF-243, BF-244, BF-245, BF-246, BF-247, BF-248, BF-249, BF-250, BF-251, BF-252, BF-253, BF-254, BF-255, BF-256, BF-257, BF-258, BF-259, BF-260, BF-261, BF-262, BF-263, BF-265, BF-266, BF-267, BF-268, BF-269, BF-270, BF-271, BF-272, BF-273, BF-274, BF-275, BF-276, BF-277, BF-278, BF-279, BF-280, BF-281, BF-282, BF-283, BF-284, BF-286, BF-287, BF-288, BF-289, BF-290, BF-291, BF-292, BF-293, BF-294, BF-295, BF-296, BF-297, BF-298, BF-299, BF-300, BF-301, BF-302, BF-303, BF-304, BF-305, BF-307, BF-308, BF-309, BF-310, BF-311, BF-312, BF-313, BF-314, BF-315, BF-317, BF-318, BF-319, BF-320, BF-321, BF-322, BF-323, BF-324, BF-325, BF-326, BF-328, BF-329, BF-330, BF-331, BF-332, BF-333, BF-334, BF-335, BF-336, BF-337, BF-338, BF-339, BF-340, BF-341, BF-342, BF-343, BF-344, BF-345, BF-346, BF-347, BF-349, BF-350, BF-353, BF-354, BF-355, BF-356, BF-357, BF-358, BF-359, BF-360, BF-361, BF-362, BF-363, BF-364, BF-365, BF-366, BF-367, BF-368, BF-369, BF-370, BF-372, BF-373, BF-374, BF-375, BF-376, BF-377 wherein the ratio of the one or more BFs relative to an Expression Reference Feature (ERF) indicates whether invasive ductal carcinoma is present. In a specific embodiment, a decrease in one or more BF/ERF ratios in a test sample relative to the BF/ERF ratios in a control sample or a reference range indicates the presence of invasive ductal carcinoma; BF-1, BF-2, BF-3, BF-4, BF-5, BF-6, BF-7, BF-8, BF-9, BF-10, BF-11, BF-12, BF-13, BF-14, BF-15, BF-16, BF-17, BF-19, BF-20, BF-22, BF-23, BF-24, BF-26, BF-73, BF-1 14, BF-233, BF-235, BF-236, BF-237, BF-238, BF-239, BF-241, BF-246, BF-251, BF-253, BF-254, BF-258, BF-260, BF-261, BF-262, BF-263, BF-264, BF-265, BF-266, BF-267, BF-268, BF-269, BF-270, BF-271, BF-272, BF-280, BF-281, BF-283, BF-290, BF-291, BF-292, BF-294, BF-295, BF-297, BF-318, BF-319, BF-321, BF-349, BF-350, BF-353, BF-354, BF-355, BF-356, BF-359, BF-360, BF-361, BF-362, BF-363, BF-364, BF-365, BF-366, BF-367, BF-371, BF-372, BF-374, BF-375, BF-376, suitable BFs for this purpose. In another specific embodiment, an increase in one or more BF/ERF ratios in a test sample relative to the BF/ERF ratios in a control sample or a reference range indicates the presence of invasive ductal carcinoma; BF-33, BF-34, BF-35, BF-36, BF-37, BF-38, BF-39, BF-40, BF-41, BF-42, BF-43, BF-44, BF-45, BF-46, BF-48, BF-49, BF-50, BF-51, BF-52, BF-53, BF-54, BF-55, BF-57, BF-60, BF-62, BF-65, BF-67, BF-71, BF-72, BF-78, BF-90, BF-99, BF-101, BF-102, BF-106, BF-122, BF-125, BF-126, BF-127, BF-131, BF-134, BF-138, BF-234, BF-240, BF-242, BF-244, BF-245, BF-247, BF-248, BF-249, BF-250, BF-252, BF-255, BF-256, BF-257, BF-259, BF-273, BF-274, BF-275, BF-276, BF-277, BF-278, BF-282, BF-284, BF-285, BF-287, BF-288, BF-289, BF-293, BF-296, BF-298, BF-299, BF-300, BF-301, BF-302, BF-303, BF-304, BF-305, BF-306, BF-307, BF-308, BF-309, BF-310, BF-311, BF-312, BF-314, BF-315, BF-316, BF-317, BF-320, BF-322, BF-323, BF-324, BF-325, BF-326, BF-327, BF-328, BF-329, BF-330, BF-331, BF-332, BF-333, BF-334, BF-335, BF-336, BF-338, BF-339, BF-340, BF-341, BF-342, BF-343, BF-344, BF-345, BF-346, BF-347, BF-348, BF-357, BF-358, BF-368, BF-369, BF-370, BF-373, BF-375, BF-377 are suitable BFs for this purpose.

In a further embodiment of the invention, breast tissue from a subject is analyzed by 2D electrophoresis for quantitative detection of (a) one or more BFs, or any combination of them, whose decreased BF/ERF ratio(s) in a test sample relative to the BF/ERF ratio(s) in a control sample indicates the presence of invasive ductal carcinoma, i.e., BF-1, BF-2, BF-3, BF-4, BF-5, BF-6, BF-7, BF-8, BF-9, BF-10, BF-1l, BF-12, BF-13, BF-14, BF-17, BF-19, BF-20, BF-21, BF-22, BF-23, BF-24, BF-26, BF-73, BF-114, BF-233, BF-235, BF-236, BF-237, BF-238, BF-239, BF-241, BF-246, BF-251, BF-253, BF-254, BF-260, BF-261, BF-262, BF-263, BF-264, BF-265, BF-266, BF-267, BF-268, BF-269, BF-271, BF-272, BF-279, BF-280, BF-281, BF-283, BF-290, BF-291, BF-292, BF-294, BF-295, BF-297, BF-318, BF-319, BF-321, BF-349, BF-350, BF-353, BF-354, BF-355, BF-356, BF-359, BF-360, BF-361, BF-362, BF-363, BF-364, BF-365, BF-366, BF-367, BF-372, BF-374, BF-376; (b) one or more BFs, or any combination of them, whose increased BF/ERF ratio(s) in a test sample relative to the BF/ERF ratio(s) in a control sample indicates the presence of invasive ductal carcinoma, i.e., BF-33, BF-34, BF-35, BF-36, BF-37, BF-38, BF-39, BF-40, BF-41, BF-42, BF-43, BF-44, BF-45, BF-46, BF-47, BF-48, BF-49, BF-51, BF-52, BF-53, BF-54, BF-55, BF-57, BF-60, BF-62, BF-64, BF-65, BF-67, BF-72, BF-78, BF-90, BF-99, BF-101, BF-102, BF-106, BF-122, BF-123, BF-125, BF-127, BF-131, BF-134, BF-138, BF-234, BF-240, BF-242, BF-243, BF-244, BF-245, BF-247, BF-248, BF-249, BF-250, BF-252, BF-255, BF-256, BF-257, BF-259, BF-273, BF-275, BF-276, BF-277, BF-278, BF-282, BF-284, BF-285, BF-286, BF-287, BF-288, BF-289, BF-293, BF-296, BF-298, BF-299, BF-300, BF-301, BF-302, BF-303, BF-304, BF-306, BF-307, BF-308, BF-309, BF-310, BF-311, BF-312, BF-313, BF-314, BF-315, BF-316, BF-317, BF-320, BF-322, BF-323, BF-324, BF-325, BF-326, BF-327, BF-328, BF-330, BF-331, BF-332, BF-333, BF-334, BF-335, BF-336, BF-337, BF-338, BF-339, BF-340, BF-341, BF-342, BF-343, BF-344, BF-345, BF-346, BF-347, BF-348, BF-357, BF-368, BF-369, BF-370, BF-373, BF-375, BF-377.

In another embodiment of the invention, cells sedimented from pleural effusions from a subject (e.g., a subject suspected of having breast cancer) are analyzed by 2D electrophoresis for quantitative detection of one or more of the following BFs: BF-1, BF-2, BF-4, BF-5, BF-7, BF-9, BF-10, BF-13, BF-14, BF-15, BF-16, BF-17, BF-19, BF-24, BF-26, BF-27, BF-31, BF-73, BF-75, BF-352, BF-353, BF-354, BF-355, BF-356, BF-359, BF-360, BF-361, BF-362, BF-363, BF-364, BF-365, BF-366, BF-367, BF-371, BF-372, BF-374, BF-376, BF-378, BF-379, BF-380, BF-381, BF-382, BF-383, BF-384, BF-386, BF-387, BF-388, BF-389, BF-390, BF-391, BF-392, BF-393, BF-395, BF-398, BF-399, BF-400, BF-405, BF-407, BF-412, BF-413, BF-415, BF-418, BF-420, BF-421. A decreased abundance of said one or more BFs in the breast tissue from the subject relative to breast tissue from a subject or subjects free from breast cancer (e.g., a control sample or a previously determined reference range) indicates the presence of metastatic breast cancer.

In another embodiment of the invention, cells sedimented from pleural effusions from a subject are analyzed by 2D electrophoresis for quantitative detection of one or more of the following BFs: BF-38, BF-39, BF-40, BF-46, BF-52, BF-56, BF-57, BF-60, BF-65, BF-67, BF-80, BF-81, BF-83, BF-84, BF-90, BF-101, BF-135, BF-351, BF-357, BF-358, BF-368, BF-369, BF-370, BF-373, BF-375, BF-377, BF-394, BF-396, BF-397, BF-401, BF-403, BF-404, BF-406, BF-408, BF-409, BF-410, BF-411, BF-414, BF-416, BF-417, BF-419. An increased abundance of said one or more BFs in the breast tissue from the subject relative to breast tissue from a subject or subjects free from breast cancer (e.g., a control sample or a previously determined reference range) indicates the presence of metastatic breast cancer.

In yet another embodiment, cells sedimented from pleural effusions from a subject are analyzed by 2D electrophoresis for quantitative detection of (a) one or more BFs or any combination of them, whose decreased abundance indicates the presence of metastatic breast cancer, i.e., BF-1, BF-2, BF-4, BF-5, BF-7, BF-9, BF-10, BF-13, BF-14, BF-15, BF-17, BF-19, BF-21, BF-22, BF-24, BF-26, BF-27, BF-31, BF-73, BF-75, BF-353, BF-354, BF-355, BF-356, BF-359, BF-360, BF-361, BF-362, BF-363, BF-364, BF-366, BF-367, BF-371, BF-372, BF-374, BF-376, BF-378, BF-379, BF-380, BF-381, BF-382, BF-383, BF-384, BF-385, BF-386, BF-387, BF-388, BF-389, BF-390, BF-391, BF-393, BF-395, BF-398, BF-399, BF-400, BF-405, BF-407, BF-412, BF-413, BF-415, BF-418, BF-420, BF-421, and (b) one or more BFs or any combination of them, whose increased abundance indicates the presence of metastatic breast cancer i.e., BF-38, BF-39, BF-40, BF-46, BF-52, BF-56, BF-57, BF-60, BF-65, BF-67, BF-80, BF-81, BF-83, BF-84, BF-101, BF-135, BF-351, BF-357, BF-358, BF-368, BF-369, BF-370, BF-373, BF-375, BF-394, BF-396, BF-397, BF-401, BF-402, BF-403, BF-404, BF-406, BF-408, BF-409, BF-410, BF-411, BF-414, BF-416, BF-417, BF-419.

In yet another embodiment of the invention, cells sedimented from pleural effusions from a subject are analyzed by 2D electrophoresis for quantitative detection of one or more of the following BFs: BF-1, BF-2, BF-4, BF-5, BF-7, BF-9, BF-10, BF-13, BF-14, BF-15, BF-16, BF-17, BF-19, BF-21, BF-22, BF-24, BF-26, BF-27, BF-31, BF-38, BF-40, BF-46, BF-52, BF-56, BF-57, BF-60, BF-65, BF-67, BF-73, BF-75, BF-80, BF-83, BF-84, BF-90, BF-101, BF-135, BF-351, BF-352, BF-353, BF-354, BF-355, BF-357, BF-358, BF-359, BF-360, BF-361, BF-362, BF-363, BF-364, BF-365, BF-366, BF-367, BF-368, BF-369, BF-370, BF-371, BF-372, BF-373, BF-374, BF-375, BF-376, BF-378, BF-379, BF-380, BF-381, BF-382, BF-383, BF-384, BF-385, BF-386, BF-387, BF-388, BF-389, BF-390, BF-391, BF-392, BF-393, BF-394, BF-395, BF-396, BF-397, BF-399, BF-400, BF-401, BF-402, BF-403, BF-404, BF-405, BF-406, BF-407, BF-408, BF-409, BF-410, BF-411, BF-412, BF-413, BF-414, BF-415, BF-416, BF-417, BF-418, BF-420, BF-421, wherein the ratio of the one or more BFs relative to an Expression Reference Feature (ERF) indicates whether metastatic breast cancer is present. In a specific embodiment, a decrease in one or more BF/ERF ratios in a test sample relative to the BF/ERF ratios in a control sample or a reference range indicates the presence of metastatic breast cancer; BF-1, BF-2, BF-4, BF-5, BF-7, BF-9, BF-10, BF-13, BF-14, BF-15, BF-16, BF-19, BF-21, BF-22, BF-24, BF-26, BF-27, BF-31, BF-73, BF-75, BF-352, BF-354, BF-355, BF-356, BF-359, BF-360, BF-361, BF-362, BF-363, BF-364, BF-365, BF-367, BF-371, BF-372, BF-374, BF-376, BF-378, BF-379, BF-380, BF-381, BF-382, BF-383, BF-384, BF-385, BF-386, BF-387, BF-388, BF-389, BF-390, BF-391, BF-392, BF-393, BF-395, BF-398, BF-399, BF-400, BF-405, BF-407, BF-412, BF-413, BF-415, BF-420, BF-421 are suitable BFs for this purpose. In another specific embodiment, an increase in one or more BF/ERF ratios in a test sample relative to the BF/ERF ratios in a control sample or a reference range indicates the presence of metastatic breast cancer; BF-38, BF-39, BF-40, BF-46, BF-52, BF-56, BF-57, BF-60, BF-65, BF-67, BF-80, BF-81, BF-83, BF-90, BF-101, BF-135, BF-351, BF-357, BF-358, BF-368, BF-369, BF-370, BF-373, BF-375, BF-377, BF-394, BF-396, BF-397, BF-401, BF-402, BF-403, BF-404, BF-406, BF-408, BF-409, BF-410, BF-411, BF-414, BF-416, BF-417, BF-419 are suitable BFs for this purpose.

In a further embodiment of the invention, cells sedimented from pleural effusions from a subject are analyzed by 2D electrophoresis for quantitative detection of (a) one or more BFs, or any combination of them, whose decreased BF/ERF ratio(s) in a test sample relative to the BF/ERF ratio(s) in a control sample indicates the presence of metastatic breast cancer, i.e., BF-1, BF-2, BF-4, BF-5, BF-7, BF-9, BF-10, BF-13, BF-14, BF-15, BF-17, BF-19, BF-21, BF-22, BF-24, BF-26, BF-27, BF-31, BF-73, BF-75, BF-352, BF-354, BF-355, BF-356, BF-359, BF-360, BF-361, BF-362, BF-363, BF-364, BF-365, BF-366, BF-367, BF-371, BF-372, BF-374, BF-376, BF-378, BF-379, BF-380, BF-381, BF-383, BF-384, BF-385, BF-386, BF-387, BF-388, BF-389, BF-390, BF-391, BF-392, BF-393, BF-395, BF-398, BF-399, BF-400, BF-405, BF-407, BF-412, BF-413, BF-415, BF-420, BF-42 1; (b) one or more BFs, or any combination of them, whose increased BF/ERF ratio(s) in a test sample relative to the BF/ERF ratio(s) in a control sample indicates the presence of metastatic breast cancer, i.e., BF-38, BF-39, BF-40, BF-46, BF-52, BF-56, BF-57, BF-60, BF-65, BF-67, BF-80, BF-81, BF-83, BF-84, BF-90, BF-101, BF-135, BF-357, BF-358, BF-368, BF-369, BF-370, BF-373, BF-375, BF-377, BF-394, BF-396, BF-397, BF-401, BF-402, BF-403, BF-404, BF-406, BF-408, BF-409, BF-410, BF-411, BF-416, BF-417, BF-419.

In another embodiment of the invention, breast tissue from a subject (e.g., a subject suspected of having breast cancer) is analyzed by 2D electrophoresis for quantitative detection of one or more of the following BFs: BF-1, BF-2, BF-3, BF-7, BF-9, BF-11, BF-15, BF-17, BF-22, BF-23, BF-25, BF-73, BF-85, BF-86, BF-114, BF-203, BF-204, BF-205, BF-207, BF-208, BF-215, BF-217, BF-218, BF-224, BF-225, BF-233, BF-235, BF-238, BF-239, BF-241, BF-246, BF-352, BF-353, BF-354, BF-355, BF-356. A decreased abundance of said one or more BFs in the breast tissue from the subject relative to breast tissue from a subject or subjects free from breast cancer (e.g., a control sample or a previously determined reference range) indicates the presence of invasive lobular carcinoma.

In another embodiment of the invention, breast tissue from a subject is analyzed by 2D electrophoresis for quantitative detection of one or more of the following BFs: BF-33, BF-34, BF-36, BF-38, BF-40, BF-42, BF-46, BF-47, BF-51, BF-52, BF-60, BF-64, BF-76, BF-83, BF-89, BF-90, BF-92, BF-94, BF-99, BF-101, BF-103, BF-106, BF-112, BF-135, BF-137, BF-201, BF-202, BF-206, BF-209, BF-210, BF-211, BF-212, BF-213, BF-214, BF-229, BF-230, BF-231, BF-232, BF-234, BF-240, BF-242, BF-243, BF-244, BF-245, BF-248, BF-249, BF-351, BF-357, BF-358. An increased abundance of said one or more BFs in the breast tissue from the subject relative to breast tissue from a subject or subjects free from breast cancer (e.g., a control sample or a previously determined reference range) indicates the presence of invasive lobular carcinoma.

In yet another embodiment, breast tissue from a subject is analyzed by 2D electrophoresis for quantitative detection of (a) one or more BFs or any combination of them, whose decreased abundance indicates the presence of invasive lobular carcinoma, i.e., BF-1, BF-2, BF-3, BF-7, BF-9, BF-11, BF-15, BF-17, BF-22, BF-23, BF-25, BF-73, BF-75, BF-76, BF-114, BF-203, BF-204, BF-205, BF-207, BF-208, BF-215, BF-217, BF-218, BF-224, BF-225, BF-233, BF-235, BF-236, BF-237, BF-238, BF-239, BF-241, BF-246, BF-354, BF-355, BF-356, and (b) one or more BFs or any combination of them, whose increased abundance indicates the presence of invasive lobular carcinoma i.e., BF-33, BF-34, BF-36, BF-38, BF-40, BF-42, BF-46, BF-47, BF-51, BF-52, BF-60, BF-64, BF-65, BF-83, BF-89, BF-90, BF-92, BF-94, BF-99, BF-101, BF-103, BF-106, BF-112, BF-123, BF-135, BF-137, BF-201, BF-202, BF-206, BF-209, BF-210, BF-211, BF-212, BF-213, BF-214, BF-216, BF-219, BF-220, BF-221, BF-222, BF-223, BF-226, BF-227, BF-228, BF-230, BF-231, BF-232, BF-234, BF-240, BF-242, BF-243, BF-244, BF-245, BF-247, BF-248, BF-249, BF-351, BF-357, BF-358.

In yet another embodiment of the invention, breast tissue from a subject is analyzed by 2D electrophoresis for quantitative detection of one or more of the following BFs: BF-1, BF-2, BF-3, BF-7, BF-9, BF-11, BF-15, BF-17, BF-22, BF-23, BF-25, BF-34, BF-36, BF-38, BF-40, BF-42, BF-46, BF-47, BF-51, BF-52, BF-60, BF-64, BF-73, BF-76, BF-83, BF-85, BF-86, BF-89, BF-90, BF-92, BF-94, BF-99, BF-101, BF-106, BF-112, BF-114, BF-123, BF-135, BF-137, BF-201, BF-202, BF-203, BF-204, BF-205, BF-206, BF-207, BF-208, BF-209, BF-210, BF-211, BF-212, BF-213, BF-214, BF-216, BF-217, BF-218, BF-219, BF-220, BF-221, BF-222, BF-223, BF-224, BF-225, BF-226, BF-227, BF-228, BF-229, BF-230, BF-231, BF-232, BF-233, BF-234, BF-235, BF-237, BF-238, BF-239, BF-240, BF-241, BF-242, BF-243, BF-244, BF-245, BF-246, BF-247, BF-248, BF-249, BF-351, BF-352, BF-353, BF-354, BF-355, BF-356, BF-357, wherein the ratio of the one or more BFs relative to an Expression Reference Feature (ERF) indicates whether invasive lobular carcinoma is present. In a specific embodiment, a decrease in one or more BF/ERF ratios in a test sample relative to the BF/ERF ratios in a control sample or a reference range indicates the presence of invasive lobular carcinoma; BF-9, BF-2, BF-3, BF-7, BF-9, BF-11, BF-15, BF-17, BF-22, BF-23, BF-25, BF-73, BF-86, BF-114, BF-203, BF-204, BF-205, BF-207, BF-208, BF-215, BF-217, BF-218, BF-225, BF-233, BF-235, BF-236, BF-237, BF-238, BF-239, BF-241, BF-246, BF-352, BF-353, BF-354, BF-355, BF-356 are suitable BFs for this purpose. In another specific embodiment, an increase in one or more BF/ERF ratios in a test sample relative to the BF/ERF ratios in a control sample or a reference range indicates the presence of invasive lobular carcinoma; BF-33, BF-34, BF-36, BF-38, BF-40, BF-42, BF-46, BF-47, BF-51, BF-52, BF-60, BF-64, BF-65, BF-76, BF-83, BF-89, BF-90, BF-92, BF-94, BF-99, BF-103, BF-106, BF-112, BF-123, BF-135, BF-137, BF-201, BF-202, BF-206, BF-209, BF-211, BF-212, BF-213, BF-214, BF-216, BF-219, BF-220, BF-221, BF-222, BF-223, BF-226, BF-227, BF-228, BF-229, BF-230, BF-231, BF-232, BF-234, BF-240, BF-242, BF-244, BF-245, BF-247, BF-248, BF-249, BF-351, BF-357, BF-358 are suitable BFs for this purpose.

In a further embodiment of the invention, breast tissue from a subject is analyzed by 2D electrophoresis for quantitative detection of (a) one or more BFs, or any combination of them, whose decreased BF/ERF ratio(s) in a test sample relative to the BF/ERF ratio(s) in a control sample indicates the presence of invasive lobular carcinoma, i.e., BF-1, BF-2, BF-3, BF-7, BF-9, BF-1 1, BF-15, BF-17, BF-22, BF-23, BF-25, BF-73, BF-85, BF-86, BF-203, BF-204, BF-205, BF-207, BF-208, BF-215, BF-217, BF-218, BF-224, BF-225, BF-233, BF-235, BF-236, BF-237, BF-238, BF-239, BF-241, BF-246, BF-352, BF-353, BF-355, BF-356; (b) one or more BFs, or any combination of them, whose increased BF/ERF ratio(s) in a test sample relative to the BF/ERF ratio(s) in a control sample indicates the presence of invasive lobular carcinoma, i.e., BF-33, BF-34, BF-36, BF-40, BF-48, BF-50, BF-51, BF-53, BF-58, BF-60, BF-64, BF-76, BF-82, BF-83, BF-87, BF-88, BF-89, BF-91, BF-92, BF-93, BF-94, BF-95, BF-96, BF-97, BF-98, BF-99, BF-100, BF-102, BF-103, BF-104, BF-105, BF-106, BF-107, BF-108, BF-109, BF-110, BF-111, BF-112, BF-139, BF-140, BF-144.

In a preferred embodiment, breast tissue from a subject is analyzed for quantitative detection of a plurality of BFs.

The methods and compositions for clinical screening, diagnosis and prognosis of breast cancer in a subject may be diagnostic of a breast cancer stage or indicative of a breast cancer stage.

Diagnostic methods and compositions are preferably based on Breast Cancer-Associated Features (BFs) and Breast Cancer-Associated Protein Isoforms (BPIs) which are specifically and particularly associated with a given breast cancer stage and are generally not associated with other breast cancer stages. Such diagnostic BFs or BPIs are useful in screening, diagnosis and prognosis as indicators of this given breast cancer stage. The administration of therapeutic compositions which are directed against or lead to modulation of diagnostic markers may have therapeutic value particularly in a given breast cancer stage.

Indicative methods and compositions are based on Breast Cancer-Associated Features (BFs) and Breast Cancer-Associated Protein Isoforms (BPIs) which are associated with a given breast cancer stage but may not be specific only for this given breast cancer stage, and may be associated with one or more other breast cancer stages. Such indicative BFs or BPIs, which are associated with a given breast cancer stage, but not only with this given breast cancer stage, are useful in screening, diagnosis and prognosis as indicators of this given breast cancer stage. Indicative methods and compositions are particularly useful in the initial or general screening, diagnosis and prognosis of an individual subject, whereby a first indication of a subset of conditions or diseases, including the given breast cancer stage, is thereby provided. Additional assessment utilizing diagnostic or particular breast cancer BFs or BPIs may then be undertaken to provide specific, diagnostic screening, diagnosis and prognosis of the individual subject. The administration of therapeutic compositions which are directed against or lead to modulation of indicative markers may have therapeutic value in a given breast cancer stage and other breast cancer stages as well, or may be useful therapeutically in more than one breast cancer stage.

Thus, a diagnostic marker changes (increases, decreases or otherwise alters form or character) significantly in only a single breast cancer stage. Such diagnostic markers are provided in Section 6 below. In particular, the diagnostic markers of the Venn diagram section A are diagnostic of Invasive Lobular Carcinoma. Moreover, the diagnostic markers of the Venn diagram section C are diagnostic of Invasive Ductal Carcinoma. Finally, the diagnostic markers of the Venn diagram section G are diagnostic of metastatic breast cancer.

An indicative marker changes (increases, decreases or otherwise alters form or character) significantly in more than one breast cancer stage. Such indicative markers are provided in Section 6 below. In particular, the indicative markers of the Veim diagram section B are indicative of Invasive Lobular Carcinoma or Invasive Ductal Carcinoma. Moreover, the indicative markers of the Venn diagram section F are indicative of Invasive Ductal Carcinoma or metastatic breast cancer. Moreover, the indicative markers of the Venn diagram section D are indicative of metastic breast cancer or Invasive Lobular Carcinoma. Finally, the indicative markers of the Venn diagram section E are indicative of Invasive Ductal Carcinoma or metastatic breast cancer or Invasive Lobular Carcinoma.

### Breast Cancer-Associated Protein Isoforms (BPIs)

In another aspect of the invention, breast tissue from a subject, preferably a living subject, is analyzed for quantitative detection of one or more Breast Cancer-Associated Protein Isoforms (BPIs) for diagnosis of breast cancer or for development of pharmaceutical products. As is well known in the art, a given protein may be expressed as variants (isoforms) that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pl, MW, or both. It follows that differential presence of a protein isoform does not require differential expression of the gene encoding the protein in question. As used herein, the term “Breast Cancer-Associated Protein Isoform” refers to a protein isoform that is differentially present in breast tissue from a subject having breast cancer compared with breast tissue from a subject free from breast cancer. As used herein, the term “isoform” also refers to a protein that exists in only a single form, i.e., it is not expressed as several variants.

Eight groups of BPIs have been identified by amino acid sequencing of BFs. BPIs were isolated, subjected to proteolysis, and analyzed by mass spectrometry using the methods and apparatus of the Preferred Technology. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.com/, and the European Molecular Biology Laboratory web site at www.mann.embl-heidelberg.de/Services/PeptideSearch/. Identification of BPIs was performed primarily using the SEQUEST search program (Eng et al., 1994, J. Am. Soc. Mass Spectrom. 5:976-989) with raw, uninterpreted tandem mass spectra of tryptic digest peptides as described in the Examples, infra. The amino acid sequences of tryptic digest peptides of these BPIs identified by tandem mass spectrometry and database searching as described in the Examples, infra, are listed in Table VI, in addition to their corresponding pIs and MWs.

TABLE VI

BPIs Identified in Subjects Having Breast Cancer

| | | | | | | |
|---|---|---|---|---|---|---|
| Table | | | | | SEQ | Fold |
| VI | | | MW | Amino Acid Sequences of | ID | Change |
| BF# | BPI# | pI | (Da) | Tryptic Digest Peptides | NO | * |

| | | | | | | |
|---|---|---|---|---|---|---|
| BF-1 | BPI-254 | 6.73 | 57363 | ISISTSGGSFR, | 1 | − |
| BF-2 | BPI-301 | 4.85 | 49013 | ASLEGNLAETENR, | 2 | |
| | | | | LLEGEDAHLTQYK, | 3 | |
| | | | | NHEEEMNALR, | 4 | |
| | | | | TMQALEIELQSQLSMK, | 5 | |
| BF-3 | BPI-1 | 6.33 | 79913 | ALQLEEER, | 6 | − |
| | | | | APDFVFYAPR, | 7 | |
| | | | | EKEELMLR, KENPLQFK, | 8, 9 | |
| BF-4 | BPI-21 | 5.45 | 53541 | GQLEALQVDGGR, | 10 | |
| | | | | QEELEAALQR, | 11 | |
| | | | | SAYGGPVGAGIR, | 12 | |
| | | | | SMQDVVEDFK, | 13 | |
| | | | | TAAENEFVVLK, | 14 | |
| BF-5 | BPI-2 | 5.59 | 337775 | ALLTLADGR, | 15 | − |
| | | | | DYPDFSPSVDAEAIQK, | 16 | |
| | | | | FTEILCLR, | 17 | |
| | | | | GAGTNEDALIEILTTR, | 18 | |
| | | | | GIGTDEFTLNR, | 19, | |
| | | | | MLISILTER, | 20, | |
| | | | | NTPAFLAER, SEIDLLDIR, | 21, | |
| | | | | SLGDDISSETSGDFR, | 22 | |
| | | | | | 23 | |
| BF-6 | BPI-302 | 5.58 | 52905 | LEAELGNMQGLVEDFK, | 24 | − |
| | | | | LEGLTDEINFLR, | 25 | |
| | | | | LSELEAALQR, QLYEEEIR, | 26, | |
| | | | | SNMDNMFESYINNLR, | 27 | |
| | | | | TEMENEFVLIK, | 28 | |
| | | | | YEELQSLAGK, | 29, | |
| | | | | | 30 | |
| BF-7 | BPI-303 | 10.52 | 48758 | IGGIGTVPVGR, | 31, | − |
| | | | | LPLQDVYK, | 32 | |
| BF-7 | BPI-304 | 10.52 | 48758 | VGAENVAIVEPSER, | 33 | − |
| | | | | YADALQEIIQER, | 34 | |
| BF-8 | BPI-3 | 5.35 | 53897 | GQLEALQVDGGR, | 10 | − |
| | | | | QEELEAALQR, | 11 | |
| | | | | SAYGGPVGAGIR, | 12 | |
| | | | | SMQDVVEDFK, | 13 | |
| BF-10 | BPI-4 | 6.51 | 56205 | AQYEEIANR, HEISEMNR, | 35, | − |
| | | | | LAELEEALQK, | 36 | |
| | | | | ISISTSGGSFR, | 37, | |
| | | | | QLDSIVGER, | 1 | |
| | | | | QNLEPLFEQYINNLR, | 38 | |
| | | | | SFSTASAITPSVSR, | 39 | |
| | | | | TTAENEFVMLK, | 40 | |
| | | | | VSLAGACGVGGYGSR, | 41 | |
| | | | | YEELQQTAGR, | 42 | |
| | | | | | 43 | |
| BF-11 | BPI-5 | 7.61 | 53837 | IWGEDLR, SYSFDEIR, | 44, | − |
| | | | | TPAGNFVTLEEGK, | 45 | |
| | | | | VYSYFECR, | 46, | |
| | | | | | 47 | |
| BF-12 | BPI-306 | 4.91 | 42632 | DYSHYYTTIQDLR, | 48 | − |
| | | | | NHEEEISTLR, | 49 | |
| | | | | QSSATSSFGGLGGGSVR, | 50 | |
| | | | | SQYEVMAEQNR, | 51 | |
| BF-13 | BPI-308 | 6.71 | 28131 | AQIFANTVDNAR, | 52 | − |
| | | | | QSVENDIHGLR, | 53 | |
| BF-14 | BPI-309 | 5.39 | 44908 | AQIFANTVDNAR, | 52, | − |
| | | | | AQYDELAR, KVIDDTNITR, | 54, | |
| | | | | QAQEYEALLNIK, | 55, | |
| | | | | QSVENDIHGLR, | 56 | |
| | | | | SLGSVQAPSYGAR, | 53 | |
| | | | | STFSTNYR, | 57 | |
| | | | | TVQSLEIDLDSMR, | 58 | |
| | | | | | 59 | |
| BF-15 | BPI-6 | 5.49 | 26295 | DYSHYYTTIQDLR, | 48, | − |
| | | | | EELAYLKK, | 60, | |
| | | | | FETEQALR, | 61, | |
| | | | | IVLQIDNAR, NHEEEISTLR, | 62, | |
| | | | | QSSATSSFGGLGGGSVR, | 49 | |
| | | | | VLDELTLAR, | 50 | |
| | | | | | 63 | |
| BF-16 | BPI-311 | 5.41 | 51333 | EYQELMNVK, | 64 | − |
| | | | | LEAELGNMQGLVEDFK, | 24 | |
| | | | | LEGLTDEINFLR, | 25, | |
| | | | | LSELEAALQR, | 26 | |
| | | | | SNMDNMFESYINNLR, | 28 | |
| | | | | YEELQSLAGK, | 30 | |
| BF-16 | BPI-312 | 5.41 | 51333 | AVFVDLEPTVIDEVR, | 65 | − |
| BF-17 | BPI-313 | 6.54 | 54317 | TAAENEFVILK, | 66 | − |
| BF-17 | BPI-314 | 6.54 | 54317 | LDPSIFESLQK, | 67, | − |
| | | | | TEVLMENFR, | 68 | |
| | | | | VEAGDVIYIEANSGAVK, | 69 | |
| BF-19 | BPI-7 | 5.41 | 54116 | AKQEELEAALQR, | 70 | − |
| | | | | GQLEALQVDGGR, | 10 | |
| | | | | QEELEAALQR, | 11 | |
| | | | | SAYGGPVGAGIR, | 12 | |
| | | | | VDALNDEINFLR, | 71, | |
| | | | | VGFLEQQNK, | 72 | |
| BF-20 | BPI-315 | 6.84 | 55209 | GMQDLVEDFK, | 73 | − |
| | | | | QNLEPLFEQYINNLR, | 39 | |
| | | | | SGFSSISVSR, | 74, | |
| | | | | TAAENEFVILK, | 66 | |
| BF-20 | BPI-316 | 6.84 | 55209 | VFVEEQVYSEFVR, | 75 | − |
| BF-21 | BPI-317 | 6.40 | 55957 | AQYEEIANR, | 35 | − |
| | | | | GLGVGFGSGGGSSSVK, | 76 | |
| | | | | QNLEPLFEQYINNLR, | 39 | |
| | | | | SFSTASAITPSVSR, | 40 | |
| | | | | TEAESWYQTK, | 77 | |
| | | | | TTAENEFVMLK, | 41 | |
| BF-21 | BPI-318 | 6.40 | 55957 | MVSSYVGENAEFER, | 78 | − |
| BF-22 | BPI-23 | 7.55 | 21761 | GELLEAIKR, | 79 | − |
| | | | | HHAAYVNNLNVTEEK, | 80 | |
| | | | | RDFGSFDK, | 81 | |
| BF-22 | BPI-24 | 7.55 | 21761 | APSWFDTGLSEMR, | 82 | − |
| | | | | QDEHGFISR, | 83 | |
| BF-23 | BPI-319 | 5.28 | 54631 | LSELEAALQR, QLYEEEIR, | 26, | − |
| | | | | SNMDNMFESYINNLR, | 27 | |
| | | | | TEMENEFVLIK, | 28 | |
| | | | | | 29 | |
| BF-23 | BPI-320 | 5.28 | 54631 | EQEELLAPADGTVELVR, | 84 | − |
| BF-24 | BPI-8 | 4.95 | 42165 | DYSHYYTTIQDLR, | 48, | − |
| | | | | FETEQALR, | 61, | |
| | | | | LEQEIATYR, | 85, | |
| | | | | IVLQIDNAR, | 62, | |
| | | | | NHEEEISTLR, | 49 | |
| | | | | QSSATSSFGGLGGGSVR, | 50 | |
| | | | | SQYEVMAEQNR, | 51 | |
| | | | | TDLEMQIEGLK, | 86 | |
| | | | | VLDELTLAR, | 87 | |
| BF-25 | BPI-205 | 4.63 | 30260 | AVTEQGAELSNEER, | 88 | − |
| | | | | DSTLIMQLLR, NLLSVAYK, | 89, | |
| | | | | YLAEVACGDDR, | 90 | |
| | | | | | 91 | |
| BF-26 | BPI-25 | 5.99 | 57554 | GLGVGFGSGGGSSSSVK, | 76 | − |
| | | | | TTAENEFVMLK, | 41 | |
| | | | | VSLAGACGVGGYGSR, | 42 | |
| BF-27 | BPI-282 | 5.73 | 52283 | GMQDLVEDFK, | 73 | − |
| BF-31 | BPI-272 | 7.23 | 56167 | ADTLTDEINFLR, | 92 | − |
| | | | | ISIGGGSCAISGGYGSR, | 93 | |
| | | | | QEIAEINR, QLDSIVGER, | 94, | |
| | | | | SGFSSISVSR, | 38 | |
| | | | | TAAENEFVTLK, | 74, | |
| | | | | | 66 | |
| BF-33 | BPI-11 | 4.67 | 47474 | FADLSEAANR, | 95, | + |
| | | | | LGDLYEEEMR, | 96, | |
| | | | | LQEEMLQR, MALDIEIATYR, | 97, | |
| | | | | NLQEAEEWYK, | 98 | |
| | | | | VELQELNDR, | 99, | |
| | | | | | 100 | |
| BF-33 | BPI-28 | 4.67 | 47474 | ELCQGLGQPGSVLR, | 101 | + |
| | | | | ELSLAGNELGDEGAR, | 102 | |
| | | | | ELTVSNNDINEAGVR, | 103 | |
| | | | | VNPALAELNLR, | 104 | |
| BF-33 | BPI-216 | 4.67 | 47474 | LKYENEVALR, | 105 | + |
| | | | | SLLEGEGSSGGGGR, | 106 | |
| BF-34 | BPI-30 | 7.12 | 32939 | DAGTIAGLNVMR, | 107 | + |
| | | | | ITPSYVAFTPEGER, | 108 | |
| | | | | VEIIANDQGNR, | 109 | |
| BF-35 | BPI-31 | 6.54 | 34373 | HLVDEPQNLIK, | 110 | + |
| BF-36 | BPI-12 | 5.50 | 22620 | ATAVVDGAFK, | 111 | + |
| | | | | EGLGPLNIPLLADVTR, | 112 | |
| | | | | GLFIIDGK, | 113 | |
| | | | | LSEDYGVLK, | 114 | |
| | | | | QITVNDLPVGR, | 115 | |
| | | | | TDEGIAYR, | 116 | |
| BF-37 | BPI-321 | 4.99 | 30472 | LQVSQQEDITK, | 117 | + |
| | | | | SLLVTELGSSR, | 118 | |
| BF-38 | BPI-33 | 5.16 | 68895 | FSLVGIGGQDLNEGNR, | 119 | + |
| | | | | LSPEELLLR, | 120, | |
| | | | | NEALIALLR, | 121 | |
| | | | | QFVTATDVVR, | 122 | |
| BF-39 | BPI-322 | 4.77 | 33587 | SYELPDGQVITIGNER, | 123 | + |
| BF-40 | BPI-257 | 6.08 | 12362 | VVAGVANALAHK, | 124 | + |
| BF-41 | BPI-323 | 5.46 | 34675 | QEYDESGPSIVHR, | 125 | + |
| | | | | SYELPDGQVITIGNER, | 126 | |
| BF-42 | BPI-34 | 4.91 | 40702 | SQYEVMAEQNR, | 51 | + |
| BF-42 | BPI-217 | 4.91 | 40702 | FEELNADLFR, | 127 | + |
| | | | | NSLESYAFNMK, | 128 | |
| | | | | SQIHDIVLVGGSTR, | 129 | |
| BF-42 | BPI-218 | 4.91 | 40702 | YEELQSLAGK, | 30 | + |
| BF-43 | BPI-222 | 5.07 | 70174 | FSLVGIGGQDLNEGNR, | 119 | + |
| | | | | LSPEELLLR, | 120, | |
| | | | | NEALIALLR, | 121 | |
| | | | | QFVTATDVVR, | 122 | |
| BF-43 | BPI-223 | 5.07 | 70174 | ALYETELADAR, | 129 | + |
| | | | | SLETENSALQLQVTER, | 130 | |
| BF-44 | BPI-35 | 4.99 | 34791 | EVDEQMLNVQNK, | 131 | + |
| | | | | FPGQLNADLR, | 132 | |
| | | | | LAVNMVPFPR, | 133 | |
| | | | | ISEQFTAMFR, | 134 | |
| BF-45 | BPI-36 | 4.84 | 13610 | AGFAGDDAPR, | 135 | + |
| BF-46 | BPI-37 | 5.34 | 29967 | GADFLVTEVENGGSLGSK, | 136 | + |
| | | | | LDIDSPPITAR, | 137 | |
| | | | | NTGIICTIGPASR, | 138 | |
| | | | | TATESFASDPILYR, | 139 | |
| BF-47 | BPI-38 | 4.60 | 37942 | KLVILEGELER, | 140 | + |
| | | | | IQLVEEELDR, | 141, | |
| | | | | LVILEGELER, | 142 | |
| | | | | QLEEELR, | 143 | |
| BF-48 | BPI-39 | ; 6.97 | 11833 | VNHVTLSQPK, | 144 | + |
| BF-49 | BPI-238 | 6.22 | 12131 | EFTPPVQAAYQK, | 145 | + |
| | | | | LLVVYPWTQR, | 146 | |
| | | | | VVAGVANALAHK, | 124 | |
| BF-50 | BPI-40 | 5.47 | 23683 | GYSFTTTAER, | 147 | + |
| BF-50 | BPI-231 | 5.47 | 23683 | ATAVVDGAFK, | 111 | + |
| | | | | EGGLGPLNIPLLADVTR, | 112 | |
| | | | | TDEGIAYR, | 116 | |
| BF-51 | BPI-324 | 5.68 | 70074 | DAFLGSFLYEYSR, | 148 | + |
| | | | | HLVDEPQNLIK, | 110 | |
| | | | | LGEYGFQNALIVR, | 149 | |
| | | | | TVMENFVAFVDK, | 150 | |
| BF-51 | BPI-325 | 5.68 | 70074 | HIVTFDGQNFK, | 151 | + |
| BF-52 | BPI-253 | 5.89 | 69864 | DVFLGMFLYEYAR, | 152 | + |
| | | | | FQNALLVR, | 153 | |
| | | | | VPQVSTPTLVEVSR, | 154 | |
| BF-53 | BPI-42 | 4.68 | 41976 | LGDLYEEEMR, | 96 | + |
| | | | | VELQELNDR, | 100 | |
| BF-62 | BPI-245 | 4.54 | 44349 | LGDLYEEEMR, | 96, | + |
| | | | | LQEEMLQR, | 97 | |
| | | | | NLQEAEEWYK, | 99 | |
| | | | | VELQELNDR, | 100 | |
| BF-65 | BPI-47 | 4.60 | 54421 | YEELQITAGR, | 155 | + |
| BF-67 | BPI-265 | 5.40 | 28445 | TVLSGGTTMYPGIADR, | 156 | + |
| BF-73 | BPI-255 | 6.65 | 36425 | DITSDTSGDFR, | 157 | − |
| | | | | FIENEEQEYVQTVK, | 158 | |
| | | | | GTDVNVFNTILTTR, | 159 | |
| | | | | TPAQFDADELR, | 160 | |
| BF-75 | BPI-280 | 8.13 | 9988 | ALNSIIDVYHK, | 161, | − |
| | | | | GNFHAVYR, | 162 | |
| BF-76 | BPI-206 | 5.04 | 28469 | LNYKPPPQK, | 163 | + |
| | | | | TLLGDGPVVTDPK, | 164 | |
| BF-80 | BPI-326 | 7.86 | 82682 | EEVGEEAIVELVENGK, | 165 | + |
| BF-81 | BPI-327 | 7.29 | 33646 | SEDFGVNEDLADSDAR, | 166 | + |
| BF-84 | BPI-274 | 5.84 | 50081 | GDYPLEAVR, | 167, | + |
| | | | | LDIDSPPITAR, | 137 | |
| | | | | TATESFASDPILYR, | 139 | |
| BF-85 | BPI-328 | 6.32 | 28158 | VTNGAFTGEISPGMIK, | 168 | − |
| | | | | VVLAYEPVWAIGTGK, | 169 | |
| BF-86 | BPI-329 | 5.39 | 29939 | SSYYMIGEQK, | 170 | − |
| BF-90 | BPI-330 | 6.70 | 32475 | IINEPTAAAIAYGLDR, | 171 | + |
| | | | | LVNHFVEEFK, | 172 | |
| | | | | TTPSYVAFTDTER, | 173 | |
| BF-94 | BPI-331 | 5.75 | 70640 | LGEYGFQNALIVR, | 149 | + |
| | | | | LVNELTEFAK, | 174 | |
| BF-101 | BPI-259 | 5.78 | 68948 | FQNALLVR, | 153 | + |
| BF-102 | BPI-332 | 8.94 | 32089 | AEDGSVIDYELIDQDAR, | 175 | + |
| | | | | DIISDTSGDFR, | 176, | |
| | | | | QDIAFAYQR, | 177 | |
| | | | | TNQELQEINR, | 178 | |
| | | | | TPAQYDASELK, | 179 | |
| BF-102 | BPI-333 | 8.94 | 32089 | LTFDSSFSPNTGK, | 180 | + |
| | | | | WTEYGLTFTEK, | 181 | |
| BF-106 | BPI-334 | 8.00 | 23764 | LPLQDVYK, | 32 | + |
| BF-112 | BPI-57 | 7.72 | 36705 | SSPVDLVTATDQK, | 182 | + |
| BF-114 | BPI-58 | 4.67 | 16800 | EAFNMIDQNR, | 183 | − |
| | | | | FTDEEVDELYR, | 184 | |
| | | | | GNFNYIEFTR, | 185 | |
| BF-122 | BPI-20 | 5.25 | 32285 | GYSFTTTAER, | 147 | + |
| | | | | SYELPDGQVITIGNER, | 123 | |
| BF-123 | BPI-64 | 5.89 | 34642 | SGFSSVSVSR, | 186 | + |
| BF-126 | BPI-335 | 7.54 | 13341 | VFVGGLSPDTSEEQIK, | 187 | + |
| BF-126 | BPI-336 | 7.54 | 13341 | IDTIEIITDR, | 188 | + |
| | | | | LFIGGLSFETTEESLR, | 189 | |
| BF-127 | BPI-65 | 4.69 | 37235 | NLQEAEEWYK, | 99 | + |
| BF-131 | BPI-68 | 5.67 | 39910 | SYELPDGQVITIGNER, | 123 | + |
| BF-134 | BPI-69 | 5.55 | 32551 | KLEVEANNAFDQYR, | 190 | + |
| | | | | LEVEANNAFDQYR, | 191 | |
| | | | | YDPPLEDGAMPSAR, | 192 | |
| BF-134 | BPI-70 | 5.55 | 32551 | SEDFGVNEDLADSDAR, | 166 | + |
| BF-138 | BPI-71 | 8.02 | 10932 | MFLSFPTTK, | 193 | + |
| | | | | TYFPHFDLSHGGAQVK, | 194 | |
| | | | | VGAHAGEYGAEALER, | 195 | |
| BF-138 | BPI-72 | 8.02 | 10932 | EFTPPVQAAYQK, | 145 | + |
| | | | | LLVVYPWTQR, | 146 | |
| | | | | VVAGVANALAHK, | 124 | |
| BF-212 | BPI-201 | 4.69 | 38828 | SQYEVMAEQNR, | 51 | + |
| BF-214 | BPI-202 | 5.77 | 36187 | SLGLSLSGGDQEDAGR, | 196 | + |
| BF-215 | BPI-203 | 5.99 | 36060 | CFIVGADNVGSK, | 197 | − |
| | | | | GHLENNPALEK, | 198 | |
| | | | | GNVGFVFTK, | 199, | |
| | | | | IIQLLDDYPK, | 200 | |
| BF-216 | BPI-204 | 6.26 | 32119 | LTEENGFWYLDQIR, | 201 | + |
| | | | | NIFNISLQR, | 202 | |
| BF-216 | BPI-283 | 6.26 | 32119 | YQETFNVIER, | 203 | + |
| BF-216 | BPI-376 | 6.26 | 32119 | IILQIDNAR, | 204 | + |
| BF-219 | BPI-207 | 5.24 | 26524 | QSGEAFVELGSEDDVK, | 205 | + |
| BF-224 | BPI-208 | 4.65 | 18637 | FTDEEVDELYR, | 184 | − |
| | | | | GNFNYIEFTR, | 185 | |
| BF-227 | BPI-209 | 5.35 | 11395 | NMINTFVPSGK, | 206 | + |
| | | | | STYPPSGPTYR, | 207 | |
| | | | | WHELMLDK, | 208 | |
| BF-228 | BPI-210 | 5.70 | 30474 | APLDIPVPDPVK, | 209 | + |
| | | | | EPALNEANLSNLK, | 210 | |
| | | | | IEDGNNFGVAVQEK, | 211 | |
| | | | | LEGFHTQISK, | 212, | |
| | | | | LMVMEIR, | 213 | |
| | | | | ISELDAFLK, | 214 | |
| | | | | QLVHELDEAEYR, | 215 | |
| BF-234 | BPI-211 | 5.77 | 96415 | NMQDMVEDYR, | 216 | + |
| BF-235 | BPI-337 | 6.92 | 56452 | QNLEPLFEQYINNLR, | 39 | − |
| | | | | SFSTASAITPSVSR, | 40 | |
| BF-236 | BPI-212 | 7.87 | 55046 | GMQDLVEDFK, | 73 | − |
| | | | | SGFSSVSVSR, | 186 | |
| BF-237 | BPI-213 | 7.77 | 54027 | AHLVAVFNEYQR, | 217 | |
| | | | | GTITDAPGFDPLR, | 218 | |
| | | | | SLYHDISGDTSGDYR, | 219 | |
| BF-238 | BPI-214 | 6.46 | 53651 | GMQDLVEDFK, | 73 | |
| | | | | ISIGGGSCAISGGYGSR, | 93 | |
| | | | | QCANLQAAIADAEQR, | 220 | |
| | | | | SGFSSISVSR, | 74 | |
| | | | | TAAENEFVTLK, | 66 | |
| BF-239 | BPI-215 | 7.52 | 48780 | AAVPSGASTGIYEALELR, | 221 | − |
| | | | | GNPTVEVDLFTSK, | 222 | |
| | | | | LAQANGWGVMVSHR, | 223 | |
| | | | | IGAEVYHNLK, | 224 | |
| | | | | VNQIGSVTESLQACK, | 225 | |
| | | | | VVIGMDVAASEFFR, | 226 | |
| | | | | YISPDQLADLYK, | 227 | |
| BF-244 | BPI-219 | 5.00 | 40576 | AQIHDLVLVGGSTR, | 228 | + |
| | | | | FELSGIPPAPR, | 229 | |
| | | | | NALESYAFNMK, | 230 | |
| BF-246 | BPI-338 | 4.61 | 17035 | AALEDTLAETEAR, | 221 | − |
| | | | | SQYEVMAEQNR, | 51 | |
| BF-248 | BPI-220 | 4.97 | 15909 | AGFAGDDAPR, | 135, | + |
| | | | | AVFPSIVGR, | 232 | |
| | | | | VAPEEHPVLLTEAPLNPK, | 233 | |
| BF-251 | BPI-339 | 7.58 | 112993 | AYLPVNESFGTADLR, | 234 | − |
| BF-256 | BPI-221 | 6.97 | 78400 | LEQEIATYR, | 85 | + |
| BF-257 | BPI-224 | 5.12 | 69570 | EITENLMATGDLDQDGR, | 235 | + |
| | | | | FSLVGIGGQDLNEGNR, | 119 | |
| | | | | GSVSDEEMMELR, | 236 | |
| BF-260 | BPI-340 | 8.59 | 58137 | DPVQEAWAEDVDLR, | 237 | − |
| BF-260 | BPI-341 | 8.59 | 58137 | THNLEPYFESFINNLR, | 238 | − |
| BF-260 | BPI-342 | 8.59 | 58137 | NYSPYYNTIDDLK, | 239 | − |
| BF-262 | BPI-343 | 8.31 | 56802 | GLGVGFGSGGGSSSSVK, | 76 | − |
| | | | | QNLEPLFEQYINNLR, | 39 | |
| BF-262 | BPI-344 | 8.31 | 56802 | GGAEQFMEETER, | 240 | − |
| | | | | LPIGDVATQYFADR, | 241 | |
| | | | | NDSVVAGGGAIEMELSK, | 242 | |
| BF-263 | BPI-225 | 8.19 | 56641 | ISISTSGGSFR, | 1, 38 | − |
| | | | | QLDSIVGER, | 40 | |
| | | | | SFSTASAITPSVSR, | 42 | |
| | | | | VSLAGACGVGGYGSR, | 43 | |
| | | | | YEELQQTAGR, | | |
| BF-265 | BPI-346 | 7.57 | 56133 | QNLEPLFEQYINNLR, | 39 | − |
| | | | | VDALMDEINFMK, | 243 | |
| BF-265 | BPI-347 | 7.57 | 56133 | VFVEEQVYSEFVR, | 75 | − |
| BF-266 | BPI-348 | 6.09 | 54734 | NMQDLVEDFK, | 244 | − |
| | | | | QNLEPLFEQYINNLR, | 39 | |
| | | | | SFSTASAITPSVSR, | 40 | |
| BF-268 | BPI-226 | 8.39 | 54326 | GMQDLVEDFK, | 73 | − |
| BF-270 | BPI-227 | 4.95 | 53870 | FPGQLNADLR, | 132 | − |
| BF-276 | BPI-349 | 9.47 | 44888 | IGGIGTVPVGR, | 31, | + |
| | | | | LPLQDVYK, | 32 | |
| BF-281 | BPI-228 | 8.05 | 35007 | AEDGSVIDYELIDQDAR, | 175 | − |
| | | | | AYTNFDAER, | 245 | |
| | | | | DIISDTSGDFR, | 176 | |
| | | | | GLGTDEDSLIEIICSR, | 246 | |
| | | | | LSLEGDHSTPPSAYGSVK, | 247 | |
| | | | | QDIAFAYQR, | 177 | |
| | | | | SYSPYDMLESIR, | 248 | |
| | | | | TNQELQEINR, | 178 | |
| BF-282 | BPI-350 | 5.97 | 35101 | GNVGFVFTK, | 199, | + |
| | | | | IIQLLDDYPK, | 200 | |
| BF-287 | BPI-229 | 6.35 | 31866 | EQHLYYQDQLLPVSR, | 249 | + |
| | | | | YHLGAYTGDDVR, | 250 | |
| BF-295 | BPI-232 | 4.71 | 19697 | ASLEGNLAETENR, | 2 | − |
| | | | | LEQEIATYR, | 85 | |
| BF-298 | BPI-233 | 4.60 | 16974 | ISEQFTAMFR, | 134 | + |
| BF-299 | BPI-352 | 6.36 | 15927 | KEDLVFIFWAPESAPLK, | 251 | + |
| | | | | YALYDATYETK, | 252 | |
| BF-302 | BPI-234 | 4.57 | 13644 | DQGTYEDYVEGLR, | 253 | + |
| | | | | EAFQLFDR, | 254 | |
| | | | | EGNGTVMGAEIR, | 255 | |
| BF-302 | BPI-235 | 4.57 | 13644 | ILDSVGIEADDDR, | 256 | + |
| BF-302 | BPI-236 | 4.57 | 13644 | AQAELVGTADEATR, | 257 | + |
| BF-303 | BPI-353 | 6.49 | 13149 | NQVALNPQNTVFDAK, | 258 | + |
| BF-303 | BPI-354 | 6.49 | 13149 | EFTPPVQAAYQK, | 145 | + |
| BF-304 | BPI-237 | 7.09 | 12765 | VVAGVANALAHK, | 124 | + |
| BF-306 | BPI-355 | 6.39 | 11989 | GFDEYMK, | 259 | + |
| BF-307 | BPI-239 | 7.57 | 11987 | EFTPPVQAAYQK, | 145 | + |
| | | | | LLVVYPWTQR, | 146 | |
| | | | | VLGAFSDGLAHLDNLK, | 260 | |
| | | | | WAGVANALAHK, | 124 | |
| BF-308 | BPI-240 | 8.13 | 11900 | EFTPPVQAAYQK, | 145 | + |
| | | | | GTFATLSELHCDK, | 261 | |
| | | | | LLVVYPWTQR, | 146 | |
| | | | | VVAGVANALAHK, | 124 | |
| BF-309 | BPI-241 | 4.91 | 11597 | ISEQFTAMFR, | 134 | + |
| BF-310 | BPI-356 | 9.64 | 11633 | VNVDAVGGEALGR, | 262 | + |
| | | | | VVAGVANALAHK, | 124 | |
| BF-310 | BPI-357 | 9.64 | 11633 | DSLLQDGEFSMDLR, | 263 | + |
| BF-312 | BPI-242 | 8.12 | 11393 | EFTPPVQAAYQK, | 145 | + |
| | | | | LLVVYPWTQR, | 146 | |
| | | | | VLGAFSDGLAHLDNLK, | 260 | |
| | | | | WAGVANALAHK, | 124 | |
| BF-313 | BPI-358 | 8.85 | 11298 | MFLSFPTTK, | 193 | + |
| | | | | TYFPHFDLSHGSAQVK, | 194 | |
| | | | | VGAHAGEYGAEALER, | 195 | |
| BF-314 | BPI-243 | 9.70 | 11115 | MFLSPTTK, | 193 | + |
| BF-314 | BPI-244 | 9.70 | 11115 | DSLLQDGEFSMDLR, | 263 | + |
| | | | | SSFYVNGLTLGGQK, | 264 | |
| | | | | STGGAPTFNVTVTK, | 265 | |
| BF-315 | BPI-359 | 4.56 | 10783 | ALEESNYELEGK, | 266 | + |
| | | | | QSLEASLAETEGR, | 267 | |
| BF-315 | BPI-360 | 4.56 | 10783 | TAAENDFVTLK, | 66 | + |
| BF-324 | BPI-246 | 4.85 | 32089 | SYELPDGQVITIGNER, | 123 | + |
| BF-329 | BPI-247 | 4.65 | 21268 | GASGIQGLAR, | 269 | + |
| | | | | NGSGTLDLEEFLR, | 270 | |
| | | | | SLDADEFR, | 271 | |
| BF-332 | BPI-248 | 7.17 | 13562 | QMEQISQFLQAAER, | 272 | + |
| | | | | TLMNLGGLAVAR, | 273 | |
| BF-334 | BPI-249 | 5.52 | 10749 | LISWYDNEFGYSNR, | 274 | + |
| BF-334 | BPI-378 | 5.52 | 10749 | LLVVYPWTQR, | 146, | + |
| | | | | VHLTPEEK, | 275 | |
| BF-338 | BPI-250 | 8.05 | 19328 | GNDISSGTVLSDYVGSGPP | 276 | + |
| | | | | K, LYTLVLTDPDAPSR, | 277 | |
| BF-342 | BPI-251 | 4.98 | 58155 | SLYASSPGGVYATR, | 278 | + |
| | | | | VELQELNDR, | 100 | |
| BF-347 | BPI-252 | 7.68 | 28267 | EFTPPVQAAYQK, | 145 | + |
| | | | | LLVVYPWTQR, | 146 | |
| | | | | VLGAFSDGLAHLDNLK, | 260 | |
| BF-349 | BPI-361 | 7.96 | 54540 | AIGGGLSSVGGGSSTIK, | 279 | − |
| | | | | SLYGLGGSK, | 280 | |
| BF-353 | BPI-362 | 7.41 | 55021 | GLGVGFGSGGGSSSSVK, | 76 | − |
| | | | | QNLEPLFEQYINNLR, | 39 | |
| | | | | SFSTASAITPSVSR, | 40 | |
| BF-353 | BPI-363 | 7.41 | 55021 | VFVEEQVYSEFVR, | 75 | − |
| BF-354 | BPI-364 | 8.65 | 54440 | QNLEPLFEQYINNLR, | 39 | − |
| | | | | TAAENEFVTLK, | 66 | |
| BF-355 | BPI-365 | 4.80 | 23721 | EVATNSELVQSGK, | 281 | − |
| | | | | TMQALEIELQSQLSMK, | 5 | |
| BF-355 | BPI-366 | 4.80 | 23721 | LAAIAESGVER, | 282 | − |
| BF-356 | BPI-256 | 4.85 | 17566 | LTMQNLNDR, | 283 | − |
| BF-357 | BPI-258 | 7.36 | 11451 | EFTPPVQAAYQK, | 145 | + |
| | | | | LLVVYPWTQR, | 146 | |
| | | | | NVDEVGGEALGR, | 284 | |
| | | | | VVAGVANALAHK, | 124 | |
| BF-359 | BPI-260 | 6.18 | 57756 | TTAENEFVMLK, | 41 | − |
| BF-360 | BPI-261 | 6.24 | 57140 | SFSTASAITPSVSR, | 40 | − |
| | | | | YEELQQTAGR, | 43 | |
| BF-361 | BPI-367 | 7.44 | 56631 | GLGVGFGSGGGSSSSVK, | 76 | − |
| | | | | QNLEPLFEQYINNLR, | 39 | |
| | | | | SFSTASAITPSVSR, | 40 | |
| BF-361 | BPI-368 | 7.44 | 56631 | VFVEEQVYSEFVR, | 75 | − |
| BF-362 | BPI-369 | 7.93 | 56475 | QNLEPLFEQYINNLR, | 39 | − |
| | | | | TAAENEFVTLK, | 66 | |
| BF-364 | BPI-262 | 6.16 | 53276 | GMQDLVEDFK, | 73 | − |
| | | | | LEGLEDALQK, | 285 | |
| | | | | ISIGGGSCAISGGYGSR, | 93 | |
| | | | | QLDSIVGER, | 38, | |
| | | | | SGFSSVSVSR, | 186 | |
| | | | | TAAENEFVTLK, | 66 | |
| BF-365 | BPI-263 | 5.85 | 52603 | GMQDLVEDFK, | 73 | − |
| BF-367 | BPI-264 | 5.63 | 50779 | GMQDLVEDFK, | 73 | − |
| BF-368 | BPI-370 | 5.10 | 30895 | SYELPDGQVITIGNER, | 123 | + |
| BF-370 | BPI-266 | 6.84 | 25632 | EIEQEAAVELSQLR, | 286 | + |
| | | | | VPDFSEYR, | 287 | |
| BF-371 | BPI-267 | 4.70 | 23016 | ASLEGNLAETENR, | 2 | − |
| | | | | CEMEQQNQEYK, | 288 | |
| | | | | LEQEIATYR, | 85 | |
| | | | | LLEGEDAHLTQYK, | 3 | |
| BF-372 | BPI-268 | 7.52 | 20516 | APSWFDTGLSEMR, | 82 | − |
| | | | | FSVNLDVK, | 289, | |
| | | | | HFSPEELK, | 290 | |
| | | | | QDEHGFISR, | 83 | |
| | | | | RPFFPFHSPSR, | 291 | |
| BF-373 | BPI-269 | 7.16 | 12109 | EFTPPVQAAYQK, | 145 | + |
| | | | | LLVVYPWTQR, | 146, | |
| | | | | VHLTPEEK, | 275 | |
| | | | | VVAGVANALAHK, | 124 | |
| BF-373 | BPI-270 | 7.16 | 12109 | EGMNIVEAMER, | 292 | + |
| | | | | FEDENFILK, | 293, | |
| | | | | VSFELFADK, | 294 | |
| BF-374 | BPI-271 | 7.64 | 54567 | GMQDLVEDFK, | 73 | − |
| BF-379 | BPI-371 | 5.24 | 117050 | TINEVENQILTR, | 295 | − |
| BF-383 | BPI-372 | 6.24 | 72679 | EQADFAIEALAK, | 296 | − |
| | | | | IMGIPEEEQMGLLR, | 297 | |
| | | | | VSHLLGINVTDFTR, | 298 | |
| BF-390 | BPI-273 | 5.65 | 53588 | ALEESNYELEGK, | 266 | − |
| BF-404 | BPI-276 | 7.25 | 22880 | YYTPTISR, | 299 | + |
| BF-404 | BPI-379 | 7.25 | 22880 | QITVNDLPVGR, | 115 | + |
| BF-405 | BPI-277 | 4.68 | 17543 | FTDEEVDELYR, | 184 | − |
| BF-408 | BPI-278 | 5.69 | 15919 | GDGPVQGIINFEQK, | 300 | + |
| | | | | HVGDLGNVTADK, | 301 | |
| BF-408 | BPI-380 | 5.69 | 15919 | ASGQAFELILSPR, | 302 | + |
| BF-409 | BPI-279 | 4.95 | 15725 | FITIFGTR, | 303, | + |
| | | | | SEIDLFNIR, | 304 | |
| | | | | YMTISGFQIEETIDR, | 305 | |
| BF-414 | BPI-281 | 8.75 | 23225 | TVAGQDAVIVLLGTR, | 306 | + |
| BF-415 | BPI-374 | 5.79 | 53955 | QNLEPLFEQYINNLR, | 39 | − |
| | | | | TAAENEFVTLK, | 66 | |

As will be evident to one of skill in the art, based upon the present description, a given BPI can be described according to the data provided for the BPI in Table VI. The BPI is a protein comprising a peptide sequence described for the BPI (preferably comprising a plurality of, more preferably all of, the peptide sequences described for that BPI) and has a pI of about the value stated for the BPI (preferably within about 10%, more preferably within about 5% still more preferably within about 1% of the stated value) and has a MW of about the value stated for that BPI (preferably within about 10%, more preferably within about 5%, still more preferably within about 1% of the stated value).

In one embodiment, breast tissue from a subject is analyzed for quantitative detection of one or more of the following BPIs: BPI-1, BPI-2, BPI-3, BPI-4, BPI-5, BPI-6, BPI-7, BPI-8, BPI-21, BPI-23, BPI-24, BPI-25, BPI-58, BPI-212, BPI-213, BPI-214, BF-215, BPI-225, BPI-226, BPI-227, BPI-228, BPI-232, BPI-254, BPI-255, BPI-256, BPI-260, BPI-261, BPI-262, BPI-263, BPI-264, BPI-267, BPI-268, BPI-271, BPI-301, BPI-302, BF-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-337, BPI-338, BPI-339, BF-340, BF-341, BPI-342, BPI-343, BPI-344, BPI-346, BPI-347, BPI-348, BPI-361, BPI-362, BPI-364, BPI-365, BPI-366, BPI-367, BPI-368, BPI-369, or any combination of them, wherein a decreased abundance of the BPI or BPIs (or any combination of them) in the breast tissue from the subject relative to breast tissue from a subject or subjects free from breast cancer (e.g., a control sample or a previously determined reference range) indicates the presence of invasive ductal carcinoma.

In another embodiment of the invention, breast tissue from a subject is analyzed for quantitative detection of one or more of the following BPIs: BPI-l 1, BPI-12, BPI-20, BPI-28, BPI-30, BPI-31, BPI-33, BPI-34, BPI-35, BPI-36, BPI-37, BPI-38, BPI-39, BPI-42, BPI-47, BPI-64, BPI-65, BPI-68, BPI-69, BPI-70, BPI-71, BPI-72, BPI-211, BPI-216, BPI-217, BPI-218, BPI-219, BPI-220, BPI-221, BPI-222, BPI-223, BPI-224, BPI-229, BPI-231, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-257, BPI-258, BPI-259, BPI-265, BPI-266, BPI-269, BPI-270, BPI-321, BPI-322, BPI-323, BPI-324, BPI-325, BPI-330, BPI-332, BPI-333, BPI-335, BPI-336, BPI-349, BPI-350, BPI-352, BPI-353, BPI-354, BPI-355, BPI-356, BPI-357, BPI-358, BPI-359, BPI-360, BPI-370, BPI-378, or any combination of them, wherein an increased abundance of the BPI or BPIs (or any combination of them) in breast tissue from the subject relative to breast tissue from a subject or subjects free from breast cancer (e.g., a control sample or a previously determined reference range) indicates the presence of invasive ductal carcinoma.

In a further embodiment, breast tissue from a subject is analyzed for quantitative detection of (a) one or more BPIs, or any combination of them, whose decreased abundance indicates the presence of invasive ductal carcinoma, i.e., BPI-1, BPI-2, BPI-3, BPI-4, BPI-5, BPI-6, BPI-7, BPI-8, BPI-21, BPI-23, BPI-24, BPI-25, BPI-58, BPI-212, BPI-213, BPI-215, BPI-225, BPI-226, BPI-227, BPI-228, BPI-232, BPI-254, BPI-255, BPI-256, BPI-260, BPI-261, BPI-262, BPI-263, BPI-264, BPI-267, BPI-268, BPI-271, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-337, BPI-338, BPI-339, BPI-340, BPI-341, BPI-342, BPI-343, BPI-344, BPI-346, BPI-347, BPI-348, BPI-361, BPI-362, BPI-363, BPI-364, BPI-365, BPI-366, BPI-367, BPI-368, BPI-369; and (b) one or more BPIs, or any combination of them, whose increased abundance indicates the presence of invasive ductal carcinoma, i.e., BPI-l 1, BPI-12, BPI-20, BPI-28, BPI-30, BPI-31, BPI-33, BPI-34, BPI-35, BPI-36, BPI-37, BPI-38, BPI-39, BPI-40, BPI-42, BPI-47, BPI-64, BPI-65, BPI-69, BPI-70, BPI-71, BPI-72, BPI-21 1, BPI-216, BPI-217, BPI-218, BPI-219, BPI-220, BPI-221, BPI-222, BPI-223, BPI-224, BPI-229, BPI-231, BPI-233, BPI-234, BPI-235, BPI-236, BPI-237, BPI-238, BPI-239, BPI-240, BPI-241, BPI-242, BPI-243, BPI-244, BPI-246, BPI-247, BPI-248, BPI-249, BPI-250, BPI-251, BPI-252, BPI-253, BPI-257, BPI-258, BF-258, BPI-259, BPI-265, BPI-266, BPI-269, BPI-270, BPI-321, BPI-322, BPI-323, BPI-324, BPI-325, BPI-330, BPI-332, BPI-333, BPI-334, BPI-335, BPI-336, BPI-349, BPI-350, BPI-352, BPI-353, BPI-354, BPI-355, BPI-356, BPI-357, BPI-358, BPI-359, BPI-360, BPI-370, BPI-378.

In another embodiment, cells sedimented from pleural effusions from a subject are analyzed for quantitative detection of one or more of the following BPIs: BPI-2, BPI-4, BPI-6, BPI-7, BPI-8, BPI-21, BPI-23, BPI-24, BPI-25, BPI-254, BPI-255, BPI-256, BPI BPI-261, BPI-262, BPI-263, BPI-264, BPI-267, BPI-268, BPI-271, BPI-272, BPI-273, BPI-277, BPI-280, BPI-282, BPI-301, BPI-303, BPI-304, BPI-308, BPI-309, BPI-311, BPI-312, BPI-313, BPI-314, BPI-317, BPI-318, BPI-362, BPI-363, BPI-364, BPI-365, BPI-366, BPI-367, BPI-368, BPI-369, BPI-371, BPI-372, BPI-374, or any combination of them, wherein a decreased abundance of the BPI or BPIs (or any combination of them) in the cells sedimented from pleural effusions from the subject relative to breast tissue from a subject or subjects free from breast cancer (e.g., a control sample or a previously determined reference range) indicates the presence of metastatic breast cancer.

In another embodiment of the invention, cells sedimented from pleural effusions from a subject are analyzed for quantitative detection of one or more of the following BPIs: BPI-33, BPI-37, BPI-47, BPI-253, BPI-257, BPI-258, BPI-259, BPI-265, BPI-266, BPI-269, BPI-270, BPI-274, BPI-276, BPI-278, BPI-279, BPI-281, BPI-322, BPI-326, BPI-327, BPI-330, BPI-370, BPI-379, BPI-380, or any combination of them, wherein an increased abundance of the BPI or BPIs (or any combination of them) in cells sedimented from pleural effusions from the subject relative to breast tissue from a subject or subjects free from breast cancer (e.g., a control sample or a previously determined reference range) indicates the presence of metastatic breast cancer.

In a further embodiment, cells sedimented from pleural effusions from a subject are analyzed for quantitative detection of (a) one or more BPIs, or any combination of them, whose decreased abundance indicates the presence of metastatic breast cancer, i.e., BPI-2, BPI-4, BPI-6, BPI-7, BPI-8, BPI-21, BPI-23, BPI-24, BPI-25, BPI-254, BPI-255, BPI-260, BPI-261, BPI-262, BPI-263, BPI-264, BPI-267, BPI-268, BPI-271, BPI-272, BPI-273, BPI-277, BPI-280, BPI-282, BPI-301, BPI-303, BPI-304, BPI-308, BPI-309, BPI-311, BPI-312, BPI-313, BPI-314, BPI-317, BPI-318, BPI-362, BPI-363, BPI-364, BPI-365, BPI-366, BPI-367, BPI-368, BPI-369, BPI-371, BPI-372, BPI-374; and (b) one or more BPIs, or any combination of them, whose increased abundance indicates the presence of metastatic breast cancer, i.e., BPI-33, BPI-37, BPI-47, BPI-253, BPI-257, BPI-258, BPI-259, BPI-265, BPI-266, BPI-269, BPI-270, BPI-274, BPI-276, BPI-278, BPI-279, BPI-281, BPI-322, BPI-326, BPI-327, BPI-330, BPI-370, BPI-379, BPI-380.

In another embodiment of the invention, breast tissue from a subject is analyzed for quantitative detection of the following BPI: BPI-1, BPI-5, BPI-6, BPI-23, BPI-24, BPI-58, BPI-203, BPI-205, BPI-208, BPI-212, BPI-213, BPI-214, BPI-215, BPI-254, BPI-255, BPI-256, BPI-301, BPI-303, BPI-304, BPI-313, BPI-314, BPI-319, BPI-320, BPI-328, BPI-329, BPI-337, BPI-338, BPI-362, BPI-363, BPI-364, BPI-365, BPI-366, wherein an decreased abundance of the BPI in breast tissue from the subject relative to breast tissue from a subject or subjects free from breast cancer (e.g., a control sample or a previously determined reference range) indicates the presence of invasive lobular carcinoma.

In another embodiment, breast tissue from a subject is analyzed for quantitative detection of one or more of the following BPIs: BPI-l 1, BPI-12, BPI-28, BPI-30, BPI-33, BPI-34, BPI-37, BPI-38, BPI-47, BPI-57, BPI-64, BPI-201, BPI-202, BPI-204, BPI-206, BPI-207, BPI-209, BPI-210, BPI-211, BPI-216, BPI-217, BPI-218, BPI-219, BPI-220, BPI-253, BPI-257, BPI-258, BPI-259, BPI-283, BPI-324, BPI-325, BPI-330, BPI-331, BPI-334, BPI-376, or any combination of them, wherein an increased abundance of the BPI or BPIs (or any combination of them) in the breast tissue from the subject relative to breast tissue from a subject or subjects free from breast cancer (e.g., a control sample or a previously determined reference range) indicates the presence of invasive lobular carcinoma.

In a further embodiment, breast tissue from a subject is analyzed for quantitative detection of (a) a BPI whose decreased abundance indicates the presence of invasive lobular carcinoma, i.e., BPI-I, BPI-5, BPI-6, BPI-23, BPI-24, BPI-58, BPI-203, BPI-205, BPI-208, BPI-212, BPI-213, BPI-214, BPI-215, BPI-254, BPI-255, BPI-256, BPI-301, BPI-303, BPI-304, BPI-313, BPI-314, BPI-319, BPI-320, BPI-328, BPI-329, BPI-337, BPI-338, BPI-362, BPI-363, BPI-364, BPI-365, BPI-366; and (b) one or more BPIs, or any combination of them, whose increased abundance indicates the presence of invasive lobular carcinoma, i.e., BPI-11, BPI-12, BPI-28, BPI-30, BPI-33, BPI-34, BPI-37, BPI-38, BPI-47, BPI-57, BPI-201, BP1-202, BPI-204, BPI-206, BPI-207, BPI-209, BPI-210, BPI-211, BPI-216, BPI-218, BPI-219, BPI-220, BPI-253, BPI-257, BPI-258, BPI-259, BPI-283, BPI-324, BPI-325, BPI-330, BPI-331, BPI-334, BPI-376.

In yet a further embodiment, breast tissue from a subject is analyzed for quantitative detection of one or more BPIs and one or more previously known biomarkers of breast cancer. In accordance with this embodiment, the abundance of each BPI and known biomarker relative to a control or reference range indicates whether a subject has breast cancer.

Preferably, the abundance of a BPI is normalized to an Expression Reference Protein Isoform (ERPI). ERPIs can be identified by partial amino acid sequencing of ERFs, which are described above, using the methods and apparatus of the Preferred Technology. The partial amino acid sequences of ERPIs, in addition to their corresponding pIs and MWs, are presented in Tables VII to X.

TABLE VII
ERPIs in Breast Tissue of Subjects Having Invasive
Ductal Carcinoma

| | | | | Amino Acid |
|---|---|---|---|---|
| | | | | Sequences of Tryptic |
| Table VII | | | MW | Digest Peptides |
| ERF# | ERPI# | pI | (Da) | (SEQ ID NO:307) |
| ERF-2 | ERPI-1 | 5.63 | 28502 | QITLNDLPVGR |

TABLE VIII
ERPIs in Pleural Effusion Cells of Subjects Having
Metastasized Breast Cancer

| Table | | | | |
|---|---|---|---|---|
| VIII | | | | Amino Acid Sequences of |
| ERF | | | | Tryptic Digest Peptides |
| # | ERPI# | pI | MW (Da) | (SEQ ID NO:308) |
| ERF-3 | ERPI-2 | 6.03 | 39797 | WIDETPPVDQPSR |

TABLE IX
ERPIs in Breast Tissue of Subjects Having Invasive
Lobular Carcinoma

| | | | | Amino Acid Sequences |
|---|---|---|---|---|
| | | | | of Tryptic Digest |
| Table IX | | | | Peptides |
| ERF# | ERPI# | pI | MW (Da) | (SEQ ID NO:166) |
| ERF-5 | ERPI-3 | 7.29 | 33646 | SEDFGVNEDLADSDAR |

TABLE X
ERPIs in Breast Tissue of Control Subjects

| | | | | Amino Acid Sequences | SEQ |
|---|---|---|---|---|---|
| Table X | | | MW | of Tryptic Digest | ID |
| ERF# | ERPI# | pI | (Da) | Peptides | NO |
| ERF-7 | ERPI-4 | 8.05 | 35007 | LSLEGDHSTPPSAYGSVK | 247 |
| | | | | AYTNFDAER, | 245 |
| | | | | QDIAFAYQR, | 177 |
| | | | | GLGTDEDSLIEIICSR, | 246 |
| | | | | TNQELQEINR, | 178 |
| | | | | DIISDTSGDFR, | 176 |
| | | | | AEDGSVIDYELIDQDAR, | 175 |
| | | | | SYSPYDMLESIR, | 248 |
| ERF-8 | ERPI-5 | 6.97 | 11833 | VNHVTLSQPK | 144 |

As shown above, the BPIs described herein include isoforms of known proteins where the isoforms were not previously known to be associated with breast cancer. For each BPI, the present invention additionally provides: (a) a preparation comprising the isolated BPI; (b) a preparation comprising one or more fragments of the BPI; and (c) antibodies that bind to said BPI, to said fragments, or both to said BPI and to said fragments. As used herein, a BPI is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein or protein isoform having a significantly different pI or MW from those of the isolated BPI, as determined by 2D electrophoresis. As used herein, a “significantly different” pI or MW is one that permits the contaminating protein to be resolved from the BPI on 2D electrophoresis, performed according to the Reference Protocol.

In one embodiment, an isolated protein is provided, said protein comprising a peptide with the amino acid sequence identified in Table VI for a BPI, said protein having a pI and MW within 10% (preferably within 5%, more preferably within 1%) of the values identified in Table VI for that BPI.

The BPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the BPIs are separated on a 2-D gel by virtue of their MWs and pls and visualized by staining the gel. In one embodiment, the BPIs are stained with a fluorescent dye and imaged with a fluorescence scanner. Sypro Red (Molecular Probes, Inc., Eugene, Oregon) is a suitable dye for this purpose. A preferred fluorescent dye is Pyridinium, 4-[2-[4-(dipentylamino)-2-trifluoromethylphenyl] ethenyl]-1-(sulfobutyl)-, inner salt. See U.S. application Ser. No. 09/412,168, filed on Oct. 5, 1999, which is incorporated herein by reference in its entirety.

Alternatively, BPIs can be detected in an immunoassay. In one embodiment, an immunoassay is performed by contacting a sample from a subject to be tested with an anti-BPI antibody under conditions such that immunospecific binding can occur if the BPI is present, and detecting or measuring the amount of any immunospecific binding by the antibody. Anti-BPI antibodies can be produced by the methods and techniques taught herein; examples of such antibodies known in the art are set forth in Table XI. These antibodies shown in Table XI are already known to bind to the protein of which the BPI is itself a family member. Preferably, the anti-BPI antibody preferentially binds to the BPI rather than to other isoforms of the same protein. In a preferred embodiment, the anti-BPI antibody binds to the BPI with at least 2-fold greater affinity, more preferably at least 5-fold greater affinity, still more preferably at least 10-fold greater affinity, than to said other isoforms of the same protein.

BPIs can be transferred from the gel to a suitable membrane (e.g. a PVDF membrane) and subsequently probed in suitable assays that include, without limitation, competitive and non-competitive assay systems using techniques such as western blots and “sandwich” immunoassays using anti-BPI antibodies as described herein, e.g., the antibodies identified in Table XI, or others raised against the BPIs of interest. The immunoblots can be used to identify those anti-BPI antibodies displaying the selectivity required to immuno-specifically differentiate a BPI from other isoforms encoded by the same gene.

TABLE XI
Known Antibodies That Recognize BPIs
or BPI-Related Polypeptides

| Table XI | | | | |
|---|---|---|---|---|
| BF# | BPI # | Antibody | Manufacturer* | Cat. No. |
| BF-21 | BPI-317 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-26 | BPI-25 | Cytokeratin 5, | ACCURATE | BMD-V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-33 | BPI-216 | Anti- | RDI RESEARCH | RDI-CBL196 |
| | | Cytokeratin | DIAGNOSTICS, | |
| | | Type 10 | INC | |
| BF-40 | BPI-257 | Hemoglobin, | ACCURATE | BMD- J16 |
| | | Goat | CHEMICAL & | |
| | | anti-Human | SCIENTIFIC | |
| | | | CORPORATION | |
| BF-49 | BPI-238 | Hemoglobin, | ACCURATE | BMD- J16 |
| | | Goat | CHEMICAL & | |
| | | anti-Human | SCIENTIFIC | |
| | | | CORPORATION | |
| BF-52 | BPI-253 | Albumin, | ACCURATE | IMS- 01-026- |
| | | Human, | CHEMICAL & | 02 |
| | | Chicken anti- | SCIENTIFIC | |
| | | | CORPORATION | |
| BF-101 | BPI-259 | Albumin, | ACCURATE | IMS- 01 -026- |
| | | Human, | CHEMICAL & | 02 |
| | | Chicken anti- | SCIENTIFIC | |
| | | | CORPORATION | |
| BF-102 | BPI-332 | Rabbit anti- | BIODESIGN | K80100R |
| | | Annexin II | INTER- | |
| | | monomer | NATIONAL | |
| BF-138 | BPI-72 | Hemoglobin, | ACCURATE | BMD- J16 |
| | | Goat | CHEMICAL & | |
| | | anti-Human | SCIENTIFIC | |
| | | | CORPORATION | |
| BF-234 | BPI-211 | Polyclonal | RDI RESEARCH | RDI- |
| | | Rabbit | DIAGNOSTICS, | CYTOK1abr |
| | | anti-Human | INC | |
| | | Cytokeratin 1 | | |
| | | (Keratin 1) | | |
| BF-235 | BPI-337 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-260 | BPI-341 | Polylconal | RDI RESEARCH | RDI- |
| | | Rabbit | DIAGNOSTICS, | CYTOK1abr |
| | | anti-Human | INC | |
| | | Cytokeratin 1 | | |
| | | (Keratin 1) | | |
| BF-260 | BPI-342 | Monoclonal | BIODESIGN | M42107M |
| | | Anti- | INTER- | |
| | | Cytokeratin | NATIONAL | |
| BF-262 | BPI-343 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-265 | BPI-346 | Cytokeratin 5, | ACCURATE | BMD-V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-266 | BPI-348 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-281 | BPI-228 | Rabbit anti- | BIODESIGN | K80100R |
| | | Annexin II | INTER- | |
| | | monomer | NATIONAL | |
| BF-303 | BPI-354 | Hemoglobin, | ACCURATE | BMD- J16 |
| | | Goat | CHEMICAL & | |
| | | anti-Human | SCIENTIFIC | |
| | | | CORPORATION | |
| BF-304 | BPI-237 | Hemoglobin, | ACCURATE | BMD- J16 |
| | | Goat | CHEMICAL & | |
| | | anti-Human | SCIENTIFIC | |
| | | | CORPORATION | |
| BF-307 | BPI-239 | Hemoglobin, | ACCURATE | BMD- J16 |
| | | Goat | CHEMICAL & | |
| | | anti-Human | SCIENTIFIC | |
| | | | CORPORATION | |
| BF-308 | BPI-240 | Hemoglobin, | ACCURATE | BMD- J16 |
| | | Goat | CHEMICAL & | |
| | | anti-Human | SCIENTIFIC | |
| | | | CORPORATION | |
| BF-312 | BPI-242 | Hemoglobin, | ACCURATE | BMD- J16 |
| | | Goat | CHEMICAL & | |
| | | anti-Human | SCIENTIFIC | |
| | | | CORPORATION | |
| BF-315 | BPI-359 | Anti- | RDI RESEARCH | RDI-CBL196 |
| | | Cytokeratin | DIAGNOSTICS, | |
| | | Type 10 | INC | |
| BF-334 | BPI-249 | Glyceral- | BIODESIGN | H86504M |
| | | dehyde-3- | INTER- | |
| | | Phosphate | NATIONAL | |
| | | Dehydrogenase | | |
| BF-334 | BPI-378 | Hemoglobin, | ACCURATE | BMD- J16 |
| | | Goat | CHEMICAL & | |
| | | anti-Human | SCIENTIFIC | |
| | | | CORPORATION | |
| BF-347 | BPI-252 | Hemoglobin, | ACCURATE | BMD- J16 |
| | | Goat | CHEMICAL & | |
| | | anti-Human | SCIENTIFIC | |
| | | | CORPORATION | |
| BF-353 | BPI-362 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-359 | BPI-260 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-360 | BPI-261 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-361 | BPI-367 | Cytokeratin 5, | ACCURATE | BMD- V1073 |
| | | 6 & 18, 56 & | CHEMICAL & | |
| | | 45 kD, | SCIENTIFIC | |
| | | Clone: LP34, | CORPORATION | |
| | | Mab | | |
| | | anti-Human | | |
| BF-373 | BPI-269 | Hemoglobin, | ACCURATE | BMD- J16 |
| | | Goat | CHEMICAL & | |
| | | anti-Human | SCIENTIFIC | |
| | | | CORPORATION | |
| BF-390 | BPI-273 | Anti- | RDI RESEARCH | RDI-CBL196 |
| | | Cytokeratin | DIAGNOSTICS, | |
| | | Type 10 | INC | |

*Further information about these antibodies can be obtained from their commercial sources at: ACCURATE CHEMICAL & SCIENTIFIC CORPORATION http://www.accuratechemical.com/; BIODESIGN INTERNATIONAL - http://www.biodesign.com/; RDI RESEARCH DIAGNOSTICS, INC - http://www.researchd.com/;SANTA CRUZ BIOTECHNOLOGY, INC - http://www.scbt.com/.

In one embodiment, binding of antibody in tissue sections can be used to detect aberrant BPI localization or an aberrant level of one or more BPIs. In a specific embodiment, antibody to a BPI can be used to assay a tissue sample (e.g., a lymph node biopsy) from a subject for the level of the BPI where an aberrant level of BPI is indicative of breast cancer. As used herein, an “aberrant level” means a level that is increased or decreased compared with the level in a subject free from breast cancer or a reference level. If desired, the comparison can be performed with a matched sample from the same subject, taken from a portion of the body not affected by breast cancer.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a BPI can be detected in a fluid sample (e.g., blood, urine, or breast tissue homogenate) by means of a two-step sandwich assay. In the first step, a capture reagent (e.g., an anti-BPI antibody) is used to capture the BPI. Examples of such antibodies known in the art are set forth in Table XI. The capture reagent can optionally be immobilized on a solid phase. In the second step, a directly or indirectly labeled detection reagent is used to detect the captured BPI. In one embodiment, the detection reagent is a lectin. Any lectin can be used for this purpose that preferentially binds to the BPI rather than to other isoforms that have the same core protein as the BPI or to other proteins that share the antigenic determinant recognized by the antibody. In a preferred embodiment, the chosen lectin binds to the BPI with at least 2-fold greater affinity, more preferably at least 5-fold greater affinity, still more preferably at least 10-fold greater affinity, than to said other isoforms that have the same core protein as the BPI or to said other proteins that share the antigenic determinant recognized by the antibody. Based on the present description, a lectin that is suitable for detecting a given BPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the BPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P1 1230-050/Pl 1230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding a BPI, a related gene, or related nucleic acid sequences or subsequences, including complementary sequences, can also be used in hybridization assays. A nucleotide encoding a BPI, or subsequences thereof comprising at least 8 nucleotides, preferably at least 12 nucleotides, and most preferably at least 15 nucleotides can be used as a hybridization probe. Hybridization assays can be used for diagnosis of conditions, disorders, or disease states, associated with aberrant expression of genes encoding BPIs. In particular, such a hybridization assay can be carried out by a method comprising contacting a subject's sample containing nucleic acid with a nucleic acid probe capable of hybridizing to a DNA or RNA that encodes a BPI, under conditions such that hybridization can occur, and detecting or measuring any resulting hybridization. Nucleotides can be used for therapy of subjects having breast cancer, as described below.

The invention also provides diagnostic kits, comprising an anti-BPI antibody. In addition, such a kit may optionally comprise one or more of the following: (1) instructions for using the anti-BPI antibody for diagnosis, prognosis, therapeutic monitoring or any combination of these applications; (2) a labeled binding partner to the antibody; (3) a solid phase (such as a reagent strip) upon which the anti-BPI antibody is immobilized; and (4) a label or insert indicating regulatory approval for diagnostic, prognostic or therapeutic use or any combination thereof. If no labeled binding partner to the antibody is provided, the anti-BPI antibody itself can be labeled with a detectable marker, e.g., a chemiluminescent, enzymatic, fluorescent, or radioactive moiety.

The invention also provides a kit comprising a nucleic acid probe capable of hybridizing to RNA encoding a BPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a BPI, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of BPIs or a plurality of nucleic acids each encoding a BPI. A kit can optionally further comprise a predetermined amount of an isolated BPI protein or a nucleic acid encoding a BPI, e.g., for use as a standard or control.

Statistical Techniques for Identifying BPIs and BPI Clusters

Uni-variate differential analysis tools, including but not limited to fold changes, Wilcoxon Rank-Sum test and T-test, are useful in identifying individual BFs or BPIs that may be diagnostically associated with breast cancer or in identifying individual BPIs that may modulate the disease process. In most cases, however, those skilled in the art will appreciate that the disease process may be associated with a combination of BFs or BPIs (and may be regulated by a combination of BPIs), rather than individual BFs and BPIs acting in isolation. The strategies for discovering such combinations of BFs and BPIs differ from those for discovering individual BFs and BPIs. In such cases, each individual BF and BPIs can be regarded as one variable and the disease can be regarded as a joint, multi-variate effect caused by interaction of these variables. The following steps can be used to identify markers from data produced by the Preferred Technology.

The first step is to identify a collection of BFs or BPIs that individually show significant association with breast cancer. Any of the tests discussed above (fold changes, wilcoxon rank sum test, etc.) can be used at this stage. Additionally, a BFs or BPIs can be subjected to a sophisticated multi-variate analysis capable of identifying clusters of BFs or BPIs which display significant multivariate associations with breast cancer. The BFs and BPIs selected and analysed in this fashion do not necessarily need to show statistical significance when subjected to univariate analysis techniques.

Linear Discriminant Analysis (LDA) is one such procedure, which can be used to detect significant association between a cluster of variables (i.e., BFs or BPIs) and breast cancer. In performing LDA, a set of weights is associated with each variable (i.e., BF or BPI) so that the linear combination of weights and the measured values of the variables can identify the disease state by discriminating between subjects having breast cancer and subjects free from breast cancer. Enhancements to the LDA allow stepwise inclusion (or removal) of variables to optimize the discriminant power of the model. The result of the LDA is therefore a cluster of BFs or BPIs which can be used, without limitation, for diagnosis, therapy or development of pharmaceutical products. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of BFs or BPIs can be identified by qualitative measures by comparing the percentage feature presence of an BF or BPI of one group of samples (e.g., samples from diseased subjects) with the percentage feature presence of an BF or BPI in another group of samples (e.g., samples from control subjects). The “percentage feature presence” of an BF or BPI is the percentage of samples in a group of samples in which the BF or BPI is detectable by the detection method of choice. For example, if an BF is detectable in 95 percent of samples from diseased subjects, the percentage feature presence of that BF in that sample group is 95 percent. If only 5 percent of samples from non-diseased subjects have detectable levels of the same BF, detection of that BF in the sample of a subject would suggest that it is likely that the subject suffers from breast cancer.

Use in Clinical Studies

The diagnostic methods and compositions of the present invention can assist in monitoring a clinical study, e.g. to evaluate drugs for therapy of breast cancer. In one embodiment, candidate molecules are tested for their ability to restore BF or BPI levels in a subject having breast cancer to levels found in subjects free from breast cancer or, in a treated subject, to preserve BF or BPI levels at or near non-breast cancer values. The levels of one or more BFs or BPIs can be assayed.

In another embodiment, the methods and compositions of the present invention are used to screen candidates for a clinical study to identify individuals having breast cancer; such individuals can then be either excluded from or included in the study or can be placed in a separate cohort for treatment or analysis. If desired, the candidates can concurrently be screened to identify individuals with breast cancer; procedures for these screens are well known in the art.

Purification of BPIs

In particular aspects, the invention provides isolated mammalian BPI, preferably human BPI, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) BPI, e.g., binding to a BPI substrate or BPI binding partner, antigenicity (binding to an anti-BPI antibody), immunogenicity, enzymatic activity and the like.

In specific embodiments, the invention provides fragments of a BPI comprising at least 5 amino acids, at least 10 amino acids, at least 50 amino acids, or at least 75 amino acids. Fragments lacking some or all of the regions of a BPI are also provided, as are proteins (e.g., fusion proteins) comprising such fragments. Nucleic acids encoding the foregoing are provided.

Once a recombinant nucleic acid which encodes the BPI, a portion of the BPI, or a precursor of the BPI is identified, the gene product can be analyzed. This is achieved by assays based on the physical or functional properties of the product, including radioactive labeling of the product followed by analysis by gel electrophoresis, immunoassay, etc.

The BPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the BPI is identified, the entire amino acid sequence of the BPI can be deduced from the nucleotide sequence of the gene coding region contained in the recombinant nucleic acid. As a result, the protein can be synthesized by standard chemical methods known in the art (e.g. see Hunkapiller et al., 1984, Nature 310:105-111).

In another alternative embodiment, native BPIs can be purified from natural sources, by standard methods such as those described above (e.g., immunoaffinity purification).

In a preferred embodiment, BPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated BPIs that can be recovered from the gel. When used in this way for preparative-scale runs, the Preferred Technology typically provides up to 100 ng, and can provide up to 1000 ng, of an isolated BPI in a single run. Those of skill in the art will appreciate that a zoom gel can be used in any separation strategy which employs gel isoelectric focusing.

The invention thus provides an isolated BPI, an isolated BPI-related polypeptide, and an isolated derivative or fragment of a BPI or a BPI-related polypeptide; any of the foregoing can be produced by recombinant DNA techniques or by chemical synthetic methods.

Isolation of DNA Encoding a BPI

Specific embodiments for the cloning of a gene encoding a BPI, are presented below by way of example and not of limitation.

The nucleotide sequences of the present invention, including DNA and RNA, and comprising a sequence encoding a BPI or a fragment thereof, or a BPI-related polypeptide, may be synthesized using methods known in the art, such as using conventional chemical approaches or polymerase chain reaction (PCR) amplification. The nucleotide sequences of the present invention also permit the identification and cloning of the gene encoding a BPI homolog or BPI ortholog including, for example, by screening cDNA libraries, genomic libraries or expression libraries.

For example, to clone a gene encoding a BPI by PCR techniques, anchored degenerate oligonucleotides (or a set of most likely oligonucleotides) can be designed for all BPI peptide fragments identified as part of the same protein. PCR reactions under a variety of conditions can be performed with relevant cDNA and genomic DNAs (e.g., from breast tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer KD, Biotechniques, 1995,1 9(4):550-2). Vectorette PCR may be performed with probes that are, for example, anchored degenerate oligonucleotides (or most likely oligonucleotides) coding for BPI peptide fragments, using as a template a genomic library or cDNA library pools.

Anchored degenerate oligonucleotides (and most likely oligonucleotides) can be designed for all BPI peptide fragments. These oligonucleotides may be labeled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding a BPI or BPI fragment of the present invention are useful for their ability to hybridize selectively with complementary stretches of genes encoding other proteins. Depending on the application, a variety of hybridization conditions may be employed to obtain nucleotide sequences at least 30%, 35%, 40%, 45%, 50%, 55%, 60%, 65%, 70%, 75%, 80%, 85%, 90%, identical, or 100% identical, to the sequence of a nucleotide encoding a BPI.

For a high degree of selectivity, relatively stringent conditions are used to form the duplexes, such as low salt or high temperature conditions. As used herein, “highly stringent conditions” means hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% fornmamide are: 42° C. for a probe which is 95 to 100% identical to the fragment of a gene encoding a BPI, 37° C. for 90 to 95% identity and 32° C. for 70 to 90% identity.

In the preparation of genomic libraries, DNA fragments are generated, some of which will encode parts or the whole of a BPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. 1, II; Ausubel F. M. et al., eds., 1989, Current Protocols in Molecular Biology, Vol. 1, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labeled degenerate oligonucleotide probes corresponding to the amino acid sequence of any peptide of the BPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Table VI above, the BPIs disclosed herein were found to correspond to isoforms of previously identified proteins encoded by genes whose sequences are publicly known. (Sequence analysis and protein identification of BPIs was carried out using the methods described in Section 6.1.14). To screen such a gene, any probe may be used that is complementary to the gene or its complement; preferably the probe is 10 nucleotides or longer, more preferably 15 nucleotides or longer. The SWISS-PROT and trEMBL databases (held by the Swiss Institute of Bioinformatics (SIB) and the European Bioinformatics Institute (EBI) which are available at http://www.expasy.chl) and the GenBank database (held by the National Institute of Health (NIH) which is available at http://www.ncbi.nlm.nih.gov/) provide protein sequences for the BPIs listed in Table VI under the following accession numbers and each sequence is incorporated herein by reference:

TABLE XII
Nucleotide sequences encoding BPIs,
BPI-Related Proteins or ERPIs

| Table XII | | Accession Numbers of |
|---|---|---|
| BF# | BPI# | Identified Sequences |
| BF-1 | BPI-254 | 88052 |
| BF-2 | BPI-301 | Q04695 |
| BF-3 | BPI-1 | P15311 |
| BF-4 | BPI-21 | AK025881.1 |
| BF-5 | BPI-2 | P12429 |
| BF-6 | BPI-302 | P05787 |
| BF-7 | BPI-303 | P04720 |
| BF-7 | BPI-304 | 4929557 |
| BF-8 | BPI-3 | AK025881.1 |
| BF-10 | BPI-4 | M28496.1 |
| BF-11 | BPI-5 | P43490 |
| BF-12 | BPI-306 | P08727 |
| BF-13 | BPI-308 | P05783 |
| BF-14 | BPI-309 | P05783 |
| BF-15 | BPI-6 | P08727 |
| BF-16 | BPI-311 | P05787 |
| BF-16 | BPI-312 | P05216 |
| BF-17 | BPI-313 | P48668, P48669 |
| BF-17 | BPI-314 | 4151525 |
| BF-19 | BPI-7 | 386851 |
| BF-20 | BPI-315 | P48666 |
| BF-20 | BPI-316 | P47895 |
| BF-21 | BPI-317 | P13647 |
| BF-21 | BPI-318 | P55809 |
| BF-22 | BPI-23 | P04179 |
| BF-22 | BPI-24 | P02511 |
| BF-23 | BPI-319 | P05787 |
| BF-23 | BPI-320 | P19971 |
| BF-24 | BPI-8 | P08727 |
| BF-25 | BPI-205 | P27348 |
| BF-26 | BPI-25 | P13647 |
| BF-27 | BPI-282 | P02538, P48666 |
| BF-31 | BPI-272 | P04259, P48669 |
| BF-33 | BPI-11 | P08670 |
| BF-33 | BPI-28 | P13489 |
| BF-33 | BPI-216 | P13645 |
| BF-34 | BPI-30 | P11021 |
| BF-35 | BPI-31 | P02769 |
| BF-36 | BPI-12 | P32119 |
| BF-37 | BPI-321 | P29218 |
| BF-38 | BPI-33 | P13796 |
| BF-39 | BPI-322 | X00351.1, X04098.1 |
| BF-40 | BPI-257 | P02023 |
| BF-41 | BPI-323 | X04098.1 |
| BF-42 | BPI-34 | P08727 |
| BF-42 | BPI-217 | P11142 |
| BF-42 | BPI-218 | P05787 |
| BF-43 | BPI-222 | P13796 |
| BF-43 | BPI-223 | P20700 |
| BF-44 | BPI-35 | P07437 |
| BF-45 | BPI-36 | X00351.1 |
| BF-46 | BPI-37 | P14618, P14786 |
| BF-47 | BPI-38 | P06468 |
| BF-48 | BPI-39 | P01884 |
| BF-49 | BPI-238 | P02023 |
| BF-50 | BPI-40 | M10277.1 |
| BF-50 | BPI-231 | P32119 |
| BF-51 | BPI-324 | P02769 |
| BF-51 | BPI-325 | P04275 |
| BF-52 | BPI-253 | P02768 |
| BF-53 | BPI-42 | P08670 |
| BF-62 | BPI-245 | P08670 |
| BF-65 | BPI-47 | 386854, P48669 |
| BF-67 | BPI-265 | 2724046 |
| BF-73 | BPI-255 | P04083 |
| BF-75 | BPI-280 | P05109 |
| BF-76 | BPI-206 | P52566 |
| BF-80 | BPI-326 | P35579 |
| BF-81 | BPI-327 | P04083 |
| BF-84 | BPI-274 | P14618 |
| BF-85 | BPI-328 | P00938 |
| BF-86 | BPI-329 | P40261 |
| BF-90 | BPI-330 | P08107 |
| BF-94 | BPI-331 | P02769 |
| BF-101 | BPI-259 | P02768 |
| BF-102 | BPI-332 | P07355 |
| BF-102 | BPI-333 | P21796 |
| BF-106 | BPI-334 | P04720 |
| BF-112 | BPI-57 | P29218 |
| BF-114 | BPI-58 | P19105 |
| BF-122 | BPI-20 | X00351.1 |
| BF-123 | BPI-64 | P02538 |
| BF-126 | BPI-335 | 7446333 |
| BF-126 | BPI-336 | P22626 |
| BF-127 | BPI-65 | P08670 |
| BF-131 | BPI-68 | X00351.1 |
| BF-134 | BPI-69 | P47756 |
| BF-134 | BPI-70 | P04083 |
| BF-138 | BPI-71 | P01922 |
| BF-138 | BPI-72 | P02023 |
| BF-212 | BPI-201 | P08727 |
| BF-214 | BPI-202 | 7023317 |
| BF-215 | BPI-203 | P05388 |
| BF-216 | BPI-204 | P18440 |
| BF-216 | BPI-283 | Q13011 |
| BF-216 | BPI-376 | 6087993 |
| BF-219 | BPI-207 | P52597 |
| BF-224 | BPI-208 | P19105 |
| BF-227 | BPI-209 | Q13510 |
| BF-228 | BPI-210 | Q06323 |
| BF-234 | BPI-211 | P04264 |
| BF-235 | BPI-337 | P13647 |
| BF-236 | BPI-212 | P02538 |
| BF-237 | BPI-213 | P50995 |
| BF-238 | BPI-214 | P48666, P48669 |
| BF-239 | BPI-215 | P06733 |
| BF-244 | BPI-219 | P08107 |
| BF-246 | BPI-338 | P08727 |
| BF-248 | BPI-220 | P02571 |
| BF-251 | BPI-339 | M19997.1 |
| BF-256 | BPI-221 | P19012 |
| BF-257 | BPI-224 | P13796 |
| BF-260 | BPI-340 | P14618 |
| BF-260 | BPI-341 | P04264 |
| BF-260 | BPI-342 | P35527 |
| BF-262 | BPI-343 | P13647 |
| BF-262 | BPI-344 | Q99832 |
| BF-263 | BPI-225 | 88052 |
| BF-265 | BPI-346 | P13647 |
| BF-265 | BPI-347 | P47895 |
| BF-266 | BPI-348 | P13647 |
| BF-268 | BPI-226 | P02538, P48666 |
| BF-270 | BPI-227 | Q13509 |
| BF-276 | BPI-349 | P04720 |
| BF-281 | BPI-228 | P07355 |
| BF-282 | BPI-350 | P05388 |
| BF-287 | BPI-229 | P20231 |
| BF-295 | BPI-232 | Q04695 |
| BF-298 | BPI-233 | P07437 |
| BF-299 | BPI-352 | P23528 |
| BF-302 | BPI-234 | P16475 |
| BF-302 | BPI-235 | P05387 |
| BF-302 | BPI-236 | P30049 |
| BF-303 | BPI-353 | P08107 |
| BF-303 | BPI-354 | P02023 |
| BF-304 | BPI-237 | P02023 |
| BF-306 | BPI-355 | Q01469 |
| BF-307 | BPI-239 | P02023 |
| BF-308 | BPI-240 | P02023 |
| BF-309 | BPI-241 | P07437 |
| BF-310 | BPI-356 | P02042 |
| BF-310 | BPI-357 | P07737 |
| BF-312 | BPI-242 | P02023 |
| BF-313 | BPI-358 | P01922 |
| BF-314 | BPI-243 | P01922 |
| BF-314 | BPI-244 | P07737 |
| BF-315 | BPI-359 | P13645 |
| BF-315 | BPI-360 | P35908 |
| BF-324 | BPI-246 | P02571 |
| BF-329 | BPI-247 | Q13938 |
| BF-332 | BPI-248 | P37802 |
| BF-334 | BPI-249 | P04406 |
| BF-334 | BPI-378 | P02023 |
| BF-338 | BPI-250 | P30086 |
| BF-342 | BPI-251 | P08670 |
| BF-347 | BPI-252 | P02023 |
| BF-349 | BPI-361 | P02538, P48666 |
| BF-353 | BPI-362 | P13647 |
| BF-353 | BPI-363 | P47895 |
| BF-354 | BPI-364 | P04259, P48669 |
| BF-355 | BPI-365 | Q04695 |
| BF-355 | BPI-366 | P28072 |
| BF-356 | BPI-256 | P08727 |
| BF-357 | BPI-258 | 6003532 |
| BF-359 | BPI-260 | P13647 |
| BF-360 | BPI-261 | P13647 |
| BF-361 | BPI-367 | P13647 |
| BF-361 | BPI-368 | P47895 |
| BF-362 | BPI-369 | P48669 |
| BF-364 | BPI-262 | P02538, P48666 |
| BF-365 | BPI-263 | P02538, P48666 |
| BF-367 | BPI-264 | P02538, P48666 |
| BF-368 | BPI-370 | M10277.1 |
| BF-370 | BPI-266 | P47985 |
| BF-371 | BPI-267 | 004695 |
| BF-372 | BPI-268 | P02511 |
| BF-373 | BPI-269 | P02023 |
| BF-373 | BPI-270 | P05092 |
| BF-374 | BPI-271 | P48666 |
| BF-379 | BPI-371 | 4826639 |
| BF-383 | BPI-372 | P35579 |
| BF-390 | BPI-273 | P13645 |
| BF-404 | BPI-276 | P49721 |
| BF-404 | BPI-379 | Q06830 |
| BF-405 | BPI-277 | P19105 |
| BF-408 | BPI-278 | P00441 |
| BF-408 | BPI-380 | P16949 |
| BF-409 | BPI-279 | P08758 |
| BF-414 | BPI-281 | P30043 |
| BF-415 | BPI-374 | P02538, P48666, P48668 |
| ERF-2 | ERPI-1 | Q13162 |
| ERF-3 | ERPI-2 | 7259793 |
| ERF-5 | ERPI-3 | P04083 |
| ERF-7 | ERPI-4 | P07355 |
| ERF-8 | ERPI-5 | P01884 |

For any BPI, degenerate probes, or probes taken from the sequences described above by accession number may be used for screening. In the case of degenerate probes, they can be constructed from the partial amino sequence information obtained from tandem mass spectra of tryptic digest peptides of the BPI. To screen such a gene, any probe may be used that is complementary to the gene or its complement; preferably the probe is 10 nucleotides or longer, more preferably 15 nucleotides or longer. When a library is screened, clones with insert DNA encoding the BPI or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridization.

In yet another aspect of the invention, clones containing nucleotide sequences encoding the entire BPI, a fragment of a BPI, a BPI-related polypeptide, or a fragment of a BPI-related polypeptide any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed BPI or BPI-related polypeptides. In one embodiment, the various anti-BPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes a BPI, a fragment of a BPI, a BPI-related polypeptide, or a fragment of a BPI-related polypeptide can be detected using DYNA Beads according to Olsvick et al., 29th ICAAC, Houston, Tex. 1989, incorporated herein by reference. Anti-BPI antibodies are crosslinked to tosylated DYNA Beads M280, and these antibody-containing beads are then contacted with colonies or plaques expressing recombinant polypeptides. Colonies or plaques expressing a BPI or BPI-related polypeptide are identified as any of those that bind the beads.

Alternatively, the anti-BPI antibodies can be nonspecifically immobilized to a suitable support, such as silica or Celite® resin. This material is then used to adsorb to bacterial colonies expressing the BPI protein or BPI-related polypeptide as described herein.

In another aspect, PCR amplification may be used to isolate from genomic DNA a substantially pure DNA (i.e., a DNA substantially free of contaminating nucleic acids) encoding the entire BPI or a part thereof. Preferably such a DNA is at least 95% pure, more preferably at least 99% pure. Oligonucleotide sequences, degenerate or otherwise, that correspond to peptide sequences of BPIs disclosed herein can be used as primers.

PCR can be carried out, e.g., by use of a Perkin-Elmer Cetus thermal cycler and Taq polymerase (Gene Amp® or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding a BPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding a BPI can also be identified by mRNA selection by nucleic acid hybridization followed by in vitro translation. In this procedure, fragments are used to isolate complementary mRNAs by hybridization. Such DNA fragments may represent available, purified DNA encoding a BPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize a BPI. A radiolabeled cDNA encoding a BPI can be synthesized using the selected mRNA (from the adsorbed polysomes) as a template. The radiolabeled mRNA or cDNA may then be used as a probe to identify the DNA fragments encoding a BPI from among other genomic DNA fragments.

Alternatives to isolating genomic DNA encoding a BPI include, but are not limited to, chemically synthesizing the gene sequence itself from a known sequence or making cDNA to the mRNA which encodes the BPI. For example, RNA for cDNA cloning of the gene encoding a BPI can be isolated from cells which express the BPI. Those skilled in the art will understand from the present description that other methods may be used and are within the scope of the invention.

Any suitable eukaryotic cell can serve as the nucleic acid source for the molecular cloning of the gene encoding a BPI. The nucleic acid sequences encoding the BPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U. K. Vol. 1, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding a BPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the BPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native BPI, nucleotide sequences encoding amino acid sequences with functionally equivalent amino acids, nucleotide sequences encoding BPIs, fragments of BPTs, BPI-related polypeptides, or fragments of BPI-related polypeptides.

In a specific embodiment, an isolated nucleic acid molecule encoding a BPI-related polypeptide can be created by introducing one or more nucleotide substitutions, additions or deletions into the nucleotide sequence of a BPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding BPIs

The nucleotide sequence coding for a BPI, a BPI analog, a BPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the BPI or its flanking regions, or the native gene encoding the BPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human BPI) is expressed. In yet another embodiment, a fragment of a BPI comprising a domain of the BPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding a BPI or fragment thereof may be regulated by a second nucleic acid sequence so that the BPI or fragment is expressed in a host transformed with the recombinant DNA molecule. For example, expression of a BPI may be controlled by any promoter or enhancer element known in the art. Promoters which may be used to control the expression of the gene encoding a BPI or a BPI-related polypeptide include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the P-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a BPI-encoding nucleic acid, one or more origins of replication, and, optionally, one or more selectable markers (e.g., an antibiotic resistance gene).

In a specific embodiment, an expression construct is made by subcloning a BPI or a BPI-related polypeptide coding sequence into the EcoRl restriction site of each of the three pGEX vectors (Glutathione S-Transferase expression vectors; Smith and Johnson, 1988, Gene 7:31-40). This allows for the expression of the BPI product or BPI-related polypeptide from the subdlone in the correct reading frame.

In mammalian host cells, a number of viral-based expression systems may be utilized. In cases where an adenovirus is used as an expression vector, the BPI coding sequence or BPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a BPI or a BPI-related polypeptide can be identified by three general approaches: (a) nucleic acid hybridization, (b) presence or absence of “marker” gene functions, and (c) expression of inserted sequences. In the first approach, the presence of a gene encoding a BPI inserted in an expression vector can be detected by nucleic acid hybridization using probes comprising sequences that are homologous to an inserted gene encoding a BPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a BPI in the vector. For example, if the gene encoding the BPI is inserted within the marker gene sequence of the vector, recombinants containing the gene encoding the BPI insert can be identified by the absence of the marker gene function. In the third approach, recombinant expression vectors can be identified by assaying the gene product (i.e., BPI) expressed by the recombinant. Such assays can be based, for example, on the physical or functional properties of the BPI in in vitro assay systems, e.g., binding with anti-BPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered BPI or BPI-related polypeptide may be controlled. Furthermore, different host cells have characteristic and specific mechanisms for the translational and post-translational processing and modification (e.g., glycosylafion, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150: 1); and hygro, which confers resistance to hygromycin (Santerre, et al., 1984, Gene 30:147) genes.

In other specific embodiments, the BPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding a BPI, a fragment of a BPI, a BPI-related polypeptide, or a fragment of a BPI-related polypeptide can be fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesizer. Both cDNA and genomic sequences can be cloned and expressed.

Domain Structure of BPIs

Domains of some BPIs are known in the art and have been described in the scientific literature. Moreover, domains of a BPI can be identified using techniques known to those of skill in the art. For example, one or more domains of a BPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, the skilled artisan can identify domains of a BPI having enzymatic or binding activity, and further can identify nucleotide sequences encoding such domains. These nucleotide sequences can then be used for recombinant expression of a BPI fragment that retains the enzymatic or binding activity of the BPI.

Based on the present description, the skilled artisan can identify domains of a BPI having enzymatic or binding activity, and further can identify nucleotide sequences encoding such domains. These nucleotide sequences can then be used for recombinant expression of BPI fragments that retain the enzymatic or binding activity of the BPI.

In one embodiment, a BPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A BPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay.

Production of Antibodies to BPIs

According to the invention a BPI, BPI analog, BPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA ) or subclass of immunoglobulin molecule.

In one embodiment, antibodies that recognize gene products of genes encoding BPIs are publicly available. For example, antibodies that recognize these BPIs and/or their isoforms include antibodies recognizing BPI-25, BPI-72, BPI-21 1, BPI-216, BPI-228, BPI-237, BPI-238, BPI-239, BPI-240, BPI-242, BPI-249, BPI-252, BPI-253, BPI-257, BPI-259, BPI-260, BPI-261, BPI-269 and BPI-273, which antibodies can be purchased from commercial sources as shown in Table XI above. In another embodiment, methods known to those skilled in the art are used to produce antibodies that recognize a BPI, a BPI analog, a BPI-related polypeptide, or a derivative or fragment of any of the foregoing.

In one embodiment of the invention, antibodies to a specific domain of a BPI are produced. In a specific embodiment, hydrophilic fragments of a BPI are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of a BPI, one may assay generated hybridomas for a product which binds to a BPI fragment containing such domain. For selection of an antibody that specifically binds a first BPI homolog but which does not specifically bind to (or binds less avidly to) a second BPI homolog, one can select on the basis of positive binding to the first BPI homolog and a lack of binding to (or reduced binding to) the second BPI homolog. Similarly, for selection of an antibody that specifically binds a BPI but which does not specifically bind to (or binds less avidly to) a different isoform of the same protein (such as a different glycoform having the same core peptide as the BPI), one can select on the basis of positive binding to the BPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to a BPI than to a different isoform or isoforms (e.g., glycoforms) of the BPI.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a BPI, a fragment of a BPI, a BPI-related polypeptide, or a fragment of a BPI-related polypeptide. In a particular embodiment, rabbit polyclonal antibodies to an epitope of a BPI or a BPI-related polypeptide can be obtained. For example, for the production of polyclonal or monoclonal antibodies, various host animals can be immunized by injection with the native or a synthetic (e.g., recombinant) version of a BPI, a fragment of a BPI, a BPI-related polypeptide, or a fragment of a BPI-related polypeptide, including but not limited to rabbits, mice, rats, etc. The Preferred Technology described herein provides isolated BPIs suitable for such immunization. If the BPI is purified by gel electrophoresis, the BPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward a BPI, a fragment of a BPI, a BPI-related polypeptide, or a fragment of a BPI-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Patent 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a BPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragrnent, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of fuill length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Trauneckeretal., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3,1994. For further details for generating bispecific antibodies see, for example, Suresh et al., Methods in Enzymology,1986, 121:210.

The invention provides functionally active fragments, derivatives or analogs of the anti-BPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the BPIs of the invention, e.g., for imaging these proteins, measuring levels thereof in appropriate physiological samples, in diagnostic methods, etc.

Expression Of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa califomica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+nitriloacetic acid-agarose columns and histidine-tagged proteins are selectively eluted with imidazole-containing buffers.

Conjugated Antibodies

In a preferred embodiment, anti-BPI antibodies or fragments thereof are conjugated to a diagnostic or therapeutic moiety. The antibodies can be used for diagnosis. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No.4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

Anti-BPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, (α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

Diagnosis of Breast Cancer

In accordance with the present invention, test samples of breast tissue, blood or urine obtained from a subject suspected of having or known to have breast cancer can be used for diagnosis. In one embodiment, a decreased abundance of one or more BFs or BPIs (or any combination of them) in a test sample relative to a control sample (from a subject or subjects free from breast cancer) or a previously determined reference range indicates the presence of breast cancer; BFs and BPIs suitable for this purpose are identified by a “−” sign in the last column in Tables I and VI, respectively, as described in detail above. In another embodiment of the invention, an increased abundance of one or more BFs or BPIs (or any combination of them) in a test sample compared to a control sample or a previously determined reference range indicates the presence of breast cancer; BFs and BPIs suitable for this purpose are identified by a “+” sign in the last column in Tables I and VI, respectively, as described in detail above. In another embodiment, the relative abundance of one or more BFs or BPIs (or any combination of them) in a test sample compared to a control sample or a previously determined reference range indicates a subtype of breast cancer (e.g., familial or sporadic breast cancer). In yet another embodiment, the relative abundance of one or more BFs or BPIs (or any combination of them) in a test sample relative to a control sample or a previously determined reference range indicates the degree or severity of breast cancer. In any of the aforesaid methods, detection of one or more BPIs described herein may optionally be combined with detection of one or more additional biomarkers for breast cancer. Any suitable method in the art can be employed to measure the level of BFs and BPIs, including but not limited to the Preferred Technology described herein, kinase assays, immunoassays to detect and/or visualize the BPIs (e.g., Western blot, immunoprecipitation followed by sodium dodecyl sulfate polyacrylamide gel electrophoresis, immunocytochemistry, etc.). In cases where a BPI has a known function, an assay for that function may be used to measure BPI expression. In a further embodiment, a decreased abundance of mRNA encoding one or more BPIs identified in Table VI by a “−” sign in the last column in a test sample relative to a control sample or a previously determined reference range indicates the presence of breast cancer. In yet a further embodiment, an increased abundance of MRNA encoding one or more BPIs identified in Table VI by a “+” sign in the last column in a test sample relative to a control sample or previously determined reference range indicates the presence of breast cancer. Any suitable hybridization assay can be used to detect BPI expression by detecting and/or visualizing mRNA encoding the BPI (e.g., Northern assays, dot blots, in situ hybridization, etc.).

In another embodiment of the invention, labeled antibodies, derivatives and analogs thereof, which specifically bind to a BPI can be used for diagnostic purposes to detect, diagnose, or monitor breast cancer. Preferably,breast cancer is detected in an animal, more preferably in a mammal and most preferably in a human.

Screening Assays

The invention provides methods for identifying agents (e.g., candidate compounds or test compounds) that bind to a BPI or have a stimulatory or inhibitory effect on the expression or activity of a BPI. The invention also provides methods of identifying agents, candidate compounds or test compounds that bind to a BPI-related polypeptide or a BPI fusion protein or have a stimulatory or inhibitory effect on the expression or activity of a BPI-related polypeptide or a BPI fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. Nos. 5,738,996; and 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i. e., bind to) a BPI, a BPI fragment (e.g. a functionally active fragment), a BPI-related polypeptide, a fragment of a BPI-related polypeptide, or a BPI fusion protein are identified in a cell-based assay system. In accordance with this embodiment, cells expressing a BPI, a fragment of a BPI, a BPI-related polypeptide, a fragment of a BPI-related polypeptide, or a BPI fusion protein are contacted with a candidate compound or a control compound and the ability of the candidate compound to interact with the BPI is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the BPI, fragment of the BPI, BPI-related polypeptide, a fragment of the BPI-related polypeptide, or a BPI fusion protein endogenously or be genetically engineered to express the BPI, fragment of the BPI, BPI-related polypeptide, a fragment of the BPI-related polypeptide, or a BPI fusion protein. In certain instances, the BPI, fragment of the BPI, BPI-related polypeptide, a fragment of the BPI-related polypeptide, or a BPI fusion protein or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a BPI and a candidate compound. The ability of the candidate compound to interact directly or indirectly with a BPI, a fragment of a BPI, a BPI-related polypeptide, a fragment of a BPI-related polypeptide, or a BPI fusion protein can be determined by methods known to those of skill in the art. For example, the interaction between a candidate compound and a BPI, a fragment of a BPI, a BPI-related polypeptide, a fragment of a BPI-related polypeptide, or a BPI fusion protein can be determined by flow cytometry, a scintillation assay, immunoprecipitation or western blot analysis.

In another embodiment, agents that interact with (i.e., bind to) a BPI, a BPI fragment (e.g., a functionally active fragment) a BPI-related polypeptide, a fragment of a BPI-related polypeptide, or a BPI fusion protein are identified in a cell-free assay system. In accordance with this embodiment, a native or recombinant BPI or fragment thereof, or a native or recombinant BPI-related polypeptide or fragment thereof, or a BPI-fusion protein or fragment thereof, is contacted with a candidate compound or a control compound and the ability of the candidate compound to interact with the BPI or BPI-related polypeptide, or BPI fusion protein is determined. If desired, this assay may be used to screen a plurality (e.g. a library) of candidate compounds. Preferably, the BPI, BPI fragment, BPI-related polypeptide, a fragment of a BPI-related polypeptide, or a BPI-fusion protein is first immobilized, by, for example, contacting the BPI, BPI fragment, BPI-related polypeptide, a fragment of a BPI-related polypeptide, or a BPI fusion protein with an immobilized antibody which specifically recognizes and binds it, or by contacting a purified preparation of the BPI, BPI fragment, BPI-related polypeptide, fragment of a BPI-related polypeptide, or a BPI fusion protein with a surface designed to bind proteins. The BPI, BPI fragment, BPI-related polypeptide, a fragment of a BPI-related polypeptide, or a BPI fusion protein may be partially or completely purified (e.g., partially or completely free of other polypeptides) or part of a cell lysate. Further, the BPI, BPI fragment, BPI-related polypeptide, a fragment of a BPI-related polypeptide may be a fusion protein comprising the BPI or a biologically active portion thereof, or BPI-related polypeptide and a domain such as glutathionine-S-transferase. Alternatively, the BPI, BPI fragment, BPI-related polypeptide, fragment of a BPI-related polypeptide or BPI fusion protein can be biotinylated using techniques well known to those of skill in the art (e.g., biotinylation kit, Pierce Chemicals; Rockford, Ill.). The ability of the candidate compound to interact with a BPI, BPI fragment, BPI-related polypeptide, a fragment of a BPI-related polypeptide, or a BPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a BPI or is responsible for the post-translational modification of a BPI. In a primary screen, a plurality (e.g., a library) of compounds are contacted with cells that naturally or recombinantly express: (i) a BPI, an isoform of a BPI, a BPI homolog a BPI-related polypeptide, a BPI fusion protein, or a biologically active fragment of any of the foregoing; and (ii) a protein that is responsible for processing of the BPI, BPI isoform, BPI homolog, BPI-related polypeptide, BPI fusion protein, or fragment in order to identify compounds that modulate the production, degradation, or post-translational modification of the BPI, BPI isoform, BPI homolog, BPI-related polypeptide, BPI fusion protein or fragment. If desired, compounds identified in the primary screen can then be assayed in a secondary screen against cells naturally or recombinantly expressing the specific BPIs of interest. The ability of the candidate compound to modulate the production, degradation or post-translational modification of a BPI, isoform, homolog, BPI-related polypeptide, or BPI fusion protein can be determined by methods known to those of skill in the art, including without limitation, flow cytometry, a scintillation assay, immunoprecipitation and western blot analysis.

In another embodiment, agents that competitively interact with (i.e., bind to) a BPI, BPI fragment, BPI-related polypeptide, a fragment of a BPI-related polypeptide, or a BPI fusion protein are identified in a competitive binding assay. In accordance with this embodiment, cells expressing a BPI, BPI fragment, BPI-related polypeptide, a fragment of a BPI-related polypeptide, or a BPI fusion protein are contacted with a candidate compound and a compound known to interact with the BPI, BPI fragment, BPI-related polypeptide, a fragment of a BPI-related polypeptide or a BPI fusion protein; the ability of the candidate compound to competitively interact with the BPI, BPI fragment, BPI-related polypeptide, fragment of a BPI-related polypeptide, or a BPI fusion protein is then determined. Alternatively, agents that competitively interact with (i.e., bind to) a BPI, BPI fragment, BPI-related polypeptide or fragment of a BPI-related polypeptide are identified in a cell-free assay system by contacting a BPI, BPI fragment, BPI-related polypeptide, fragment of a BPI-related polypeptide, or a BPI fusion protein with a candidate compound and a compound known to interact with the BPI, BPI-related polypeptide or BPI fusion protein. As stated above, the ability of the candidate compound to interact with a BPI, BPI fragment, BPI-related polypeptide, a fragment of a BPI-related polypeptide, or a BPI fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of a BPI, or a BPI-related polypeptide are identified by contacting cells (e.g., cells of prokaryotic origin or eukaryotic origin) expressing the BPI, or BPI-related polypeptide with a candidate compound or a control compound (e.g., phosphate buffered saline (PBS)) and determining the expression of the BPI, BPI-related polypeptide, or BPI fusion protein, mRNA encoding the BPI, or mRNA encoding the BPI-related polypeptide. The level of expression of a selected BPI, BPI-related polypeptide, mRNA encoding the BPI, or mRNA encoding the BPI-related polypeptide in the presence of the candidate compound is compared to the level of expression of the BPI, BPI-related polypeptide, mRNA encoding the BPI, or mRNA encoding the BPI-related polypeptide in the absence of the candidate compound (e.g., in the presence of a control compound). The candidate compound can then be identified as a modulator of the expression of the BPI, or a BPI-related polypeptide based on this comparison. For example, when expression of the BPI or mRNA is significantly greater in the presence of the candidate compound than in its absence, the candidate compound is identified as a stimulator of expression of the BPI or mRNA. Alternatively, when expression of the BPI or mRNA is significantly less in the presence of the candidate compound than in its absence, the candidate compound is identified as an inhibitor of the expression of the BPI or mRNA. The level of expression of a BPI or the mRNA that encodes it can be determined by methods known to those of skill in the art. For example, mRNA expression can be assessed by Northern blot analysis or RT-PCR, and protein levels can be assessed by western blot analysis.

In another embodiment, agents that modulate the activity of a BPI, or a BPI-related polypeptide are identified by contacting a preparation containing the BPI or BPI-related polypeptide, or cells (e.g., prokaryotic or eukaryotic cells) expressing the BPI or BPI-related polypeptide with a test compound or a control compound and determining the ability of the test compound to modulate (e.g., stimulate or inhibit) the activity of the BPI or BPI-related polypeptide. The activity of a BPI or a BPI-related polypeptide can be assessed by detecting induction of a cellular signal transduction pathway of the BPI or BPI-related polypeptide (e.g., intracellular Ca2+, diacylglycerol, IP3, etc.), detecting catalytic or enzymatic activity of the target on a suitable substrate, detecting the induction of a reporter gene ( e.g., a regulatory element that is responsive to a BPI or a BPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate compound can then be identified as a modulator of the activity of a BPI or BPI-related polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of a BPI or BPI-related polypeptide are identified in an animal model. Examples of suitable animals include, but are not limited to, mice, rats, rabbits, monkeys, guinea pigs, dogs and cats. Preferably, the animal used represent a model of breast cancer (e.g., animal models of breast cancer include, but are not limited to xenografts of human breast cancer cell lines such as MDA-MB-435 in estrogen deprived Severe Combined Immunodeficient (SCID) mice (Eccles et al., 1994 Cell Biophysics 24(25):279). ). In accordance with this embodiment, the test compound or a control compound is administered (e.g., orally, rectally or parenterally such as intraperitoneally or intravenously) to a suitable animal and the effect on the expression, activity or both expression and activity of the BPI or BPI-related polypeptide is determined. Changes in the expression of a BPI or BPI-related polypeptide can be assessed by the methods outlined above.

In yet another embodiment, a BPI or BPI-related polypeptide is used as a “bait protein” in a two-hybrid assay or three hybrid assay to identify other proteins that bind to or interact with a BPI or BPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the BPIs of the invention as, for example, upstream or downstream elements of a signaling pathway involving the BPIs of the invention.

This invention further provides novel agents identified by the above-described screening assays and uses thereof for treatments as described herein.

Therapeutic Uses of BPIs

The invention provides for treatment of various diseases and disorders by administration of a therapeutic compound. Such compounds include but are not limited to: BPIs, BPI analogs, BPI-related polypeptides and derivatives (including fragments) thereof; antibodies to the foregoing; nucleic acids encoding BPIs, BPI analogs, BPI-related polypeptides and fragments thereof; antisense nucleic acids to a gene encoding a BPI or BPI-related polypeptide; and modulator of a gene encoding a BPI or BPI-related polypeptide. An important feature of the present invention is the identification of genes encoding BPIs involved in breast cancer. Breast cancer can be treated (e.g. to ameliorate symptoms or to retard onset or progression) or prevented by administration of a therapeutic compound that promotes function or expression of one or more BPIs that are decreased in the breast tissue of subjects having breast cancer, or by administration of a therapeutic compound that reduces function or expression of one or more BPIs that are increased in the breast tissue of subjects having breast cancer.

In one embodiment, one or more antibodies each specifically binding to a BPI are administered alone or in combination with one or more additional therapeutic compounds or treatments. Examples of such therapeutic compounds or treatments include, but are not limited to, taxol, cyclophosphamide, tamoxifen, fluorouracil and doxorubicin.

Preferably, a biological product such as an antibody is allogeneic to the subject to which it is administered. In a preferred embodiment, a human BPI or a human BPI-related polypeptide, a nucleotide sequence encoding a human BPI or a human BPI-related polypeptide, or an antibody to a human BPI or a human BPI-related polypeptide, is administered to a human subject for therapy (e.g. to ameliorate symptoms or to retard onset or progression) or prophylaxis.

Treatment And Prevention Of Breast Cancer

Breast cancer is treated or prevented by administration to a subject suspected of having or known to have breast cancer or to be at risk of developing breast cancer of a compound that modulates (i.e., increases or decreases) the level or activity (i.e., function) of one or more BPIs or the level of one or more BFs that are differentially present in the breast tissue of subjects having breast cancer compared with breast tissue of subjects free from breast cancer. In one embodiment, breast cancer is treated or prevented by administering to a subject suspected of having or known to have breast cancer or to be at risk of developing breast cancer a compound that upregulates (i.e., increases) the level or activity (i.e., function) of one or more BPIs or the level of one or more BFs that are decreased in the breast tissue of subjects having breast cancer. In another embodiment, a compound is administered that downregulates the level or activity (i.e., function) of one or more BPIs or the level of one or more BFs that are increased in the breast tissue of subjects having breast cancer. Examples of such a compound include but are not limited to: BPIs, BPI fragments and BPI-related polypeptides; nucleic acids encoding a BPI, a BPI fragment and a BPI-related polypeptide (e.g., for use in gene therapy); and, for those BPIs or BPI-related polypeptides with enzymatic activity, compounds or molecules known to modulate that enzymatic activity. Other compounds that can be used, e.g., BPI agonists, can be identified using in vitro assays.

Breast cancer is also treated or prevented by administration to a subject suspected of having or known to have breast cancer or to be at risk of developing breast cancer of a compound that downregulates the level or activity of one or more BPIs or the level of one or more BFs that are increased in the breast tissue of subjects having breast cancer. In another embodiment, a compound is administered that upregulates the level or activity of one or more BPIs or the level of one or more BFs that are decreased in the breast tissue of subjects having breast cancer. Examples of such a compound include, but are not limited to, BPI antisense oligonucleotides, ribozymes, antibodies directed against BPIs, and compounds that inhibit the enzymatic activity of a BPI. Other useful compounds e.g., BPI antagonists and small molecule BPI antagonists, can be identified using in vitro assays.

In a preferred embodiment, therapy or prophylaxis is tailored to the needs of an individual subject. Thus, in specific embodiments, compounds that promote the level or function of one or more BPIs, or the level of one or more BFs, are therapeutically or prophylactically administered to a subject suspected of having or known to have breast cancer, in whom the levels or functions of said one or more BPIs, or levels of said one or more BFs, are absent or are decreased relative to a control or normal reference range. In further embodiments, compounds that promote the level or function of one or more BPIs, or the level of one or more BFs, are therapeutically or prophylactically administered to a subject suspected of having or known to have breast cancer in whom the levels or functions of said one or more BPIs, or levels of said one or more BFs, are increased relative to a control or to a reference range. In further embodiments, compounds that decrease the level or function of one or more BPIs, or the level of one or more BFs, are therapeutically or prophylactically administered to a subject suspected of having or known to have breast cancer in whom the levels or functions of said one or more BPIs, or levels of said one or more BFs, are increased relative to a control or to a reference range. In further embodiments, compounds that decrease the level or function of one or more BPIs, or the level of one or more BFs, are therapeutically or prophylactically administered to a subject suspected of having or known to have breast cancer in whom the levels or functions of said one or more BPIs, or levels of said one or more BFs, are decreased relative to a control or to a reference range. The change in BPI function or level, or BF level, due to the administration of such compounds can be readily detected, e.g., by obtaining a sample (e.g., a sample of breast tissue, blood or urine or a tissue sample such as biopsy tissue) and assaying in vitro the levels of said BFs or the levels or activities of said BPIs, or the levels of mRNAs encoding said BPIs. or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the breast cancer BPI or BF profile towards normal with the proviso that such compounds do not include taxol, cyclophosphamide, tamoxifen, fluorouracil and doxorubicin.

Gene Therapy

In a specific embodiment, nucleic acids comprising a sequence encoding a BPI, a BPI fragment, BPI-related polypeptide or fragment of a BPI-related polypeptide, are administered to promote BPI function by way of gene therapy. Gene therapy refers to administration to a subject of an expressed or expressible nucleic acid. In this embodiment, the nucleic acid produces its encoded polypeptide that mediates a therapeutic effect by promoting BPI function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the compound comprises a nucleic acid encoding a BPI or fragment or chimeric protein thereof, said nucleic acid being part of an expression vector that expresses a BPI or fragment or chimeric protein thereof in a suitable host. In particular, such a nucleic acid has a promoter operably linked to the BPI coding region, said promoter being inducible or constitutive (and, optionally, tissue-specific). In another particular embodiment, a nucleic acid molecule is used in which the BPI coding sequences and any other desired sequences are flanked by regions that promote homologous recombination at a desired site in the genome, thus providing for intrachromosomal expression of the BPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); W092/20316 dated Nov. 26, 1992 (Findeis et al.); W093/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains a nucleic acid encoding a BPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the BPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirms vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO 94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord, blood, peripheral blood or fetal liver.

In a preferred embodiment, the cell used for gene therapy is autologous to the subject that is treated.

In an embodiment in which recombinant cells are used in gene therapy, a nucleic acid encoding a BPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Piftelkow and Scoff, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Direct injection of a DNA coding for a BPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a BPI and (b) a promoter are injected into a subject to elicit an immune response to the BPI.

Inhibition of BPIs to Treat Breast Cancer

In one embodiment of the invention, breast cancer is treated or prevented by administration of a compound that antagonizes (inhibits) the level(s) and/or function(s) of one or more BPIs which are elevated in the breast tissue of subjects having breast cancer as compared with breast tissue of subjects free from breast cancer. Compounds useful for this purpose include but are not limited to anti-BPI antibodies (and fragments and derivatives containing the binding region thereof), BPI antisense or ribozyme nucleic acids, and nucleic acids encoding dysfunctional BPIs that are used to “knockout” endogenous BPI function by homologous recombination (see, e.g., Capecchi, 1989, Science 244:1288-1292). Other compounds that inhibit BPI function can be identified by use of known in vitro assays, e.g., assays for the ability of a test compound to inhibit binding of a BPI to another protein or a binding partner, or to inhibit a known BPI function. Preferably such inhibition is assayed in vitro or in cell culture, but genetic assays may also be employed. The Preferred Technology can also be used to detect levels of the BPIs before and after the administration of the compound. Preferably, suitable in vitro or in vivo assays are utilized to determine the effect of a specific compound and whether its administration is indicated for treatment of the affected tissue, as described in more detail below.

In a specific embodiment, a compound that inhibits a BPI function is administered therapeutically or prophylactically to a subject in whom an increased breast tissue level or functional activity of the BPI (e.g., greater than the normal level or desired level) is detected as compared with breast tissue of subjects free from breast cancer or a predetermined reference range. Methods standard in the art can be employed to measure the increase in a BPI level or function, as outlined above. Preferred BPI inhibitor compositions include small molecules, i.e., molecules of 1000 daltons or less. Such small molecules can be identified by the screening methods described herein.

Antisense Regulation of BPIs

In a specific embodiment, BPI expression is inhibited by use of BPI antisense nucleic acids. The present invention provides the therapeutic or prophylactic use of nucleic acids comprising at least six nucleotides that are antisense to a gene or cDNA encoding a BPI or a portion thereof. As used herein, a BPI “antisense” nucleic acid refers to a nucleic acid capable of hybridizing by virtue of some sequence complementarity to a portion of an RNA (preferably mRNA) encoding a BPI. The antisense nucleic acid may be complementary to a coding and/or noncoding region of an mRNA encoding a BPI. Such antisense nucleic acids have utility as compounds that inhibit BPI expression, and can be used in the treatment of breast cancer.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the BPI antisense nucleic acids of the invention in a pharmaceutically acceptable carrier, as described infra.

In another embodiment, the invention provides methods for inhibiting the expression of a BPI nucleic acid sequence in a prokaryotic or eukaryotic cell comprising providing the cell with an effective amount of a composition comprising a BPI antisense nucleic acid of the invention. BPI antisense nucleic acids and their uses are described in detail below.

BPI Antisense Nucleic Acids

The BPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a BPI antisense oligonucleotide is provided, preferably of single-stranded DNA. The oligonucleotide may be modified at any position on its structure with substituents generally known in the art.

The BPI antisense oligonucleotide may comprise at least one of the following modified base moieties: 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w,2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formnacetal.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the BPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the BPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the BPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding a BPI, preferably a human gene encoding a BPI. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.) with the RNA, forming a stable duplex; in the case of double-stranded BPI antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding a BPI it may contain and still form a stable duplex (or triplex, as the case may be). One skilled in the art can ascertain a tolerable degree of mismatch by use of standard procedures to determine the melting point of the hybridized complex.

Therapeutic Use of BPI Antisense Nucleic Acids

The BPI antisense nucleic acids can be used to treat or prevent breast cancer when the target BPI is overexpressed in the breast tissue of subjects suspected of having or suffering from breast cancer. In a preferred embodiment, a single-stranded DNA antisense BPI oligonucleotide is used.

Cell types which express or overexpress RNA encoding a BPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with a BPI-specific nucleic acid (e.g., by Northern hybridization, dot blot hybridization, in situ hybridization), observing the ability of RNA from the cell type to be translated in vitro into a BPI, immunoassay, etc. In a preferred aspect, primary tissue from a subject can be assayed for BPI expression prior to treatment, e.g., by immunocytochemistry or in situ hybridization.

Pharmaceutical compositions of the invention, comprising an effective amount of a BPI antisense nucleic acid in a pharmaceutically acceptable carrier, can be administered to a subject having breast cancer. The amount of BPI antisense nucleic acid which will be effective in the treatment of breast cancer can be determined by standard clinical techniques.

In a specific embodiment, pharmaceutical compositions comprising one or more BPI antisense nucleic acids are administered via liposomes, microparticles, or microcapsules. In various embodiments of the invention, such compositions may be used to achieve sustained release of the BPI antisense nucleic acids.

Inhibitory Ribozyme andtriple Helix Approaches

In another embodiment, symptoms of breast cancer may be ameliorated by decreasing the level of a BPI or BPI activity by using gene sequences encoding the BPI in conjunction with well-known gene “knock-out,” ribozyme or triple helix methods to decrease gene expression of a BPI. In this approach ribozyme or triple helix molecules are used to modulate the activity, expression or synthesis of the gene encoding the BPI, and thus to ameliorate the symptoms of breast cancer. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding a BPI can be used to prevent translation of target gene MRNA and, therefore, expression of the gene product. (See, e.g., PCT International Publication W090/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding a BPI, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the BPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470-475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the BPI.

As in the antisense approach, the ribozymes can be composed of modified oligonucleotides (e.g., for improved stability, targeting, etc.) and should be delivered to cells that express the BPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the BPI and inhibit translation. Because ribozymes, unlike antisense molecules, are catalytic, a lower intracellular concentration is required for efficacy.

Endogenous BPI expression can also be reduced by inactivating or “knocking out” the gene encoding the BPI, or the promoter of such a gene, using targeted homologous recombination (e.g., see Smithies, et al., 1985, Nature 317:230-234; Thomas and Capecchi, 1987, Cell 51:503-512; Thompson et al., 1989, Cell 5:313-321; and Zijlstra etal., 1989, Nature 342:435-438, each of which is incorporated by reference herein in its entirety). For example, a mutant gene encoding a non-functional BPI (or a completely unrelated DNA sequence) flanked by DNA homologous to the endogenous gene (either the coding regions or regulatory regions of the gene encoding the BPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, the endogenous expression of a gene encoding a BPI can be reduced by targeting deoxyribonucleotide sequences complementary to the regulatory region of the gene (i.e., the gene promoter and/or enhancers) to form triple helical structures that prevent transcription of the gene encoding the BPI in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815). 269. Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of a BPI that the situation may arise wherein the concentration of BPI present may be lower than is necessary for a normal phenotype. In such cases, to ensure that substantially normal levels of activity of a gene encoding a BPI are maintained, gene therapy may be used to introduce into cells nucleic acid molecules that encode and express the BPI that exhibit normal gene activity and that do not contain sequences susceptible to whatever antisense, ribozyme, or triple helix treatments are being utilized. Alternatively, in instances whereby the gene encodes an extracellular protein, normal BPIs can be co-administered in order to maintain the requisite level of BPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Assays For Therapeutic Or Prophylactic Compounds

The present invention also provides assays for use in drug discovery in order to identify or verify the efficacy of compounds for treatment of breast cancer. Test compounds can be assayed for their ability to restore BF or BPI levels in a subject having breast cancer towards levels found in subjects free from breast cancer or to produce similar changes in experimental animal models of breast cancer. Compounds able to restore BF or BPI levels in a subject having breast cancer towards levels found in subjects free from breast cancer or to produce similar changes in experimental animal models of breast cancer can be used as lead compounds for further drug discovery, or used therapeutically. BF and BPI expression can be assayed by the Preferred Technology, immunoassays, gel electrophoresis followed by visualization, detection of BPI activity, or any other method taught herein or known to those skilled in the art. Such assays can be used to screen candidate drugs, in clinical monitoring or in development of pharmaceutical products, where abundance of an BF or BPI can serve as a surrogate marker for clinical disease.

In various specific embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used Examples of animal models of breast cancer include, but are not limited to, xenografts of human breast cancer cell lines such as MDA-MB-435 in estrogen deprived Severe Combined Immunodeficient (SCID) mice (Eccles et al., 1994 Cell Biophysics 24(25):279). It is also apparent to the skilled artisan that, based upon the present disclosure, transgenic animals can be produced with “knock-out” mutations of the gene or genes encoding one or more BPIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of a BPI are identified in non-human animals (e.g., mice, rats, monkeys, rabbits, and guinea pigs), preferably non-human animal models for breast cancer, expressing the BPI. In accordance with this embodiment, a test compound or a control compound is administered to the animals, and the effect of the test compound on expression of one or more BPIs is determined. A test compound that alters the expression of a BPI (or a plurality of BPIs) can be identified by comparing the level of the selected BPI or BPIs (or mRNA(s) encoding the same) in an animal or group of animals treated with a test compound with the level of the BPI(s) or mRNA(s) in an animal or group of animals treated with a control compound. Techniques known to those of skill in the art can be used to determine the MRNA and protein levels, for example, in situ hybridization. The animals may or may not be sacrificed to assay the effects of a test compound.

In another embodiment, test compounds that modulate the activity of a BPI or a biologically active portion thereof are identified in non-human animals (e.g., mice, rats, monkeys, rabbits, and guinea pigs), preferably non-human animal models for breast cancer, expressing the BPIs. In accordance with this embodiment, a test compound or a control compound is administered to the animals, and the effect of a test compound on the activity of a BPI is determined. A test compound that alters the activity of a BPI (or a plurality of BPIs) can be identified by assaying animals treated with a control compound and animals treated with the test compound. The activity of the BPI can be assessed by detecting induction of a cellular second messenger of the BPI (e.g., intracellular Ca2+, diacylglycerol, IP3, etc.), detecting catalytic or enzymatic activity of the BPI or binding partner thereof, detecting the induction of a reporter gene (e.g., a regulatory element that is responsive to a BPI of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of a BPI (see, e.g., U.S. Pat. No. 5,401,639, which is incorporated herein by reference).

In yet another embodiment, test compounds that modulate the level or expression of a BPI (or plurality of BPIs) are identified in human subjects having breast cancer, most preferably those having severe breast cancer. In accordance with this embodiment, a test compound or a control compound is administered to the human subject, and the effect of a test compound on BPI expression is determined by analyzing the expression of the BPI or the mRNA encoding the same in a biological sample (e.g., breast tissue, blood, or urine). A test compound that alters the expression of a BPI can be identified by comparing the level of the BPI or mRNA encoding the same in a subject or group of subjects treated with a control compound to that in a subject or group of subjects treated with a test compound. Alternatively, alterations in the expression of a BPI can be identified by comparing the level of the BPI or mRNA encoding the same in a subject or group of subjects before and after the administration of a test compound. Techniques known to those of skill in the art can be used to obtain the biological sample and analyze the mRNA or protein expression. For example, the Preferred Technology described herein can be used to assess changes in the level of a BPI.

In another embodiment, test compounds that modulate the activity of a BPI (or plurality of BPIs) are identified in human subjects having breast cancer, most preferably those with severe breast cancer. In this embodiment, a test compound or a control compound is administered to the human subject, and the effect of a test compound on the activity of a BPI is determined. A test compound that alters the activity of a BPI can be identified by comparing biological samples from subjects treated with a control compound to samples from subjects treated with the test compound. Alternatively, alterations in the activity of a BPI can be identified by comparing the activity of a BPI in a subject or group of subjects before and after the administration of a test compound. The activity of the BPI can be assessed by detecting in a biological sample (e.g., breast tissue, blood, or urine) induction of a cellular signal transduction pathway of the BPI (e.g., intracellular Ca2+, diacylglycerol, IP3, etc.), catalytic or enzymatic activity of the BPI or a binding partner thereof, or a cellular response, for example, cellular differentiation, or cell proliferation. Techniques known to those of skill in the art can be used to detect changes in the induction of a second messenger of a BPI or changes in a cellular response. For example, RT-PCR can be used to detect changes in the induction of a cellular second messenger.

In a preferred embodiment, a test compound that changes the level or expression of a BPI towards levels detected in control subjects (e.g., humans free from breast cancer) is selected for further testing or therapeutic use. In another preferred embodiment, a test compound that changes the activity of a BPI towards the activity found in control subjects (e.g., humans free from breast cancer) is selected for further testing or therapeutic use.

In another embodiment, test compounds that reduce the severity of one or more symptoms associated with breast cancer are identified in human subjects having breast cancer, most preferably subjects with severe breast cancer. In accordance with this embodiment, a test compound or a control compound is administered to the subjects, and the effect of a test compound on one or more symptoms of breast cancer is determined. A test compound that reduces one or more symptoms can be identified by comparing the subjects treated with a control compound to the subjects treated with the test compound. Techniques known to physicians familiar with breast cancer can be used to determine whether a test compound reduces one or more symptoms associated with breast cancer. For example, RT-PCR can be used to detect changes in the induction of a cellular second messenger.

In a preferred embodiment, a test compound that reduces the severity of one or more symptoms associated with breast cancer in a human having breast cancer is selected for further testing or therapeutic use.

Therapeutic and Prophylactic Compositions and Their Use

The invention provides methods of treatment comprising administering to a subject an effective amount of a compound of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into breast tissue or at the site (or former site) of a malignant tumor or neoplastic or pre-neoplastic tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) 288. In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the lymph node, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of breast cancer can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLE

### Identification of Proteins Differentially Expressed In The Breast Tissue In Breast Cancer

Using the following procedure, proteins in breast tissue samples were separated by isoelectric focusing followed by SDS-PAGE and analyzed. Parts 6.1.1 to 6.1.14 (inclusive) of the procedure set forth are hereby designated as the “Reference Protocol”

Materials and Methods

Sample Preparation. Tissues used in this study consisted of 14 samples of reduction mammoplasties carried out for cosmetic reasons from patients in whom no malignancy or pathology was detected; 18 samples of invasive ductal carcinoma; 6 samples of cells sedimented from pleural effusions of patients having metastatic breast cancer; 4 samples of invasive lobular carcinoma. Purified populations of normal breast luminal epithelial cells, or breast cancer cells of luminal epithelial origin were prepared as described. Clarke et al. (1994) Epithelial Cell Biol. 3:38-46. Certain modifications were included to enhance purity. After establishment of primary epithelial cultures, overnight treatment in calcium-free medium resulted in suspensions enriched in cells of luminal origin. These were incubated for 40 minutes on ice with a mixture of a rat monoclonal antibody (ICR-2, 10 mg/ml) against the luminal epithelial marker EMA, and a mouse monoclonal antibody (DAKO, clone SS2/36, 1:25) against the myoepithelial antigen CD-10. The cells were then washed in L-15/10% FCS and labelled for 15 minutes with anti-rat MACS magnetic beads (Miltenyi Biotech Inc), and positively labelled cells separated using a Vario-MACS high field intensity magnet. To obtain a pure population of cells bearing luminal markers, they were further incubated for 20 minutes with anti-mouse Dynbeads (Dynal, UK) which binds to any residual CD-10 +ve myoepithelial cells, and separated using an MPC-10 low intensity magnet, which does not attract MACS-bead labelled cells. Using these procedures, purified cells were obtained in yields of 5×106−2×107 cells from primary epithelial cultures. Purity of the resulting cell preparations was assessed by staining for cell-type specific filament proteins (cytokeratins and vimentin) as originally described by using FACS sorted cells. O'Hare et al (1991) Differentiation 46:209-221. The purified cell populations were subsequently washed five times in L-15 medium (serum-free), flash-frozen, and stored at −80° C.

400 microl of the following buffer was then added to each sample: 2M Thiourea (BDH 10348 3D); 8M urea (BDH 45204 3W); 4% CHAPS (Sigma C3023) 65 mM dithiotheitol (DTT); 2% (v/v) Resolytes 3.5-10 (BDH 44338 2×). This mixture was vortexed, centrifuged at 13000 rpm for 5 mins at 15° C., and the supernatant aspirated and stored at −80° C. An assay for protein content was carried out on the final sample (Pierce BCA Cat #23225).

Isoelectric Focusing. Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, #18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS35OOXL power supply (Cat 19-3500-01): Initial voltage=300V for 2 hrs; Linear Ramp from 300V to 3500V over 3 hrs; Hold at 3500V for 19 hrs. For all stages of the process, the current limit was set to 10 mA for 12 gels, and the wattage limit to 5W. The temperature was held at 20° C. throughout the run.

Gel Equilibration and SDS-PAGE. After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma 1-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

Preparation of supported gels. The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia Cat. #17-1330-01). The front plate was treated with (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The front and back plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored individually at 4° C. in sealed polyethylene bags with 6 ml of gel buffer, and were used within 4 weeks.

SDS-PAGE. A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining. Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016×), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl] ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/I; the staining solution was vacuum filtered through a 0.4 μm filter (Duropore) before use.

Imaging of the gel. A computer-readable output was produced by imaging the fluorescently stained gels with the Apollo 2 scanner (Oxford Glycosciences, Oxford, UK) described in section 5.1, supra. This scanner has a gel carrier with three integral fluorescent markers (Designated M1, M2, M3) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

Digital Analysis of the Data. The data were processed as described in U.S. Pat. No 6,064,654, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters: Smooths=1; Laplacian threshold 40; Partials threshold 15; Saturation=100; Peakedness=0; Minimum Perimeter=10.

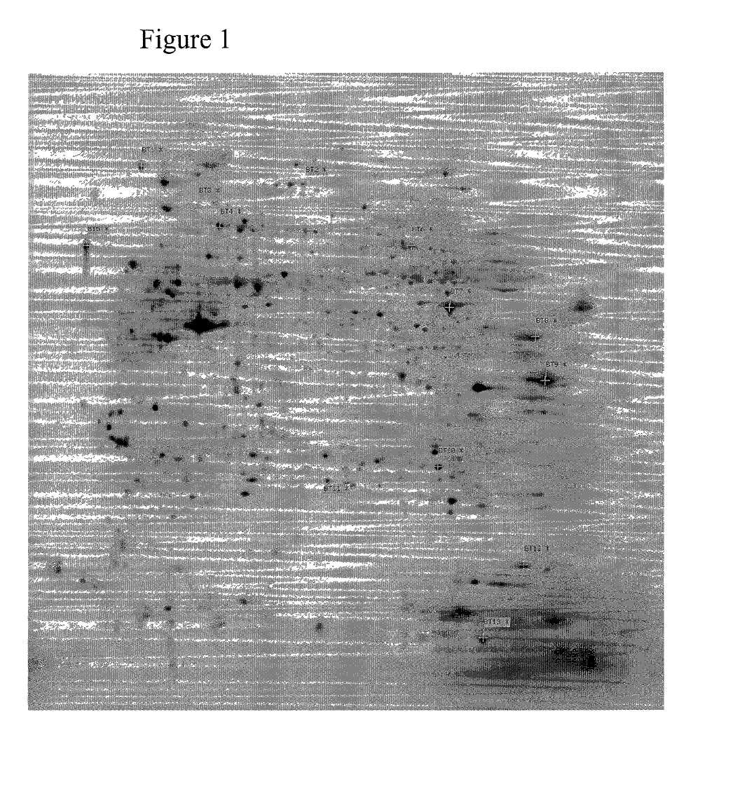

Assignment of pl and MW Values. Landmark identification was used to determine the pI and MW of features detected in the images. Thirteen landmark features, designated BT1, BT2, BT3, BT4, BT5, BT6, BT7, BT8, BT9, BTIO, BT11, BT12, an were identified in a standard breast tissue image. These landmark features are identified in FIG. 1 and were assigned the p1 and/or MW values identified in Table XIII.

TABLE XIII
Landmark Features Used In This Study

| Table XIII | | |
|---|---|---|
| Landmark | | |
| name | pI | MW (Da) |
| BT1 | 4.78 | 116528 |
| BT2 | 5.85 | 104386 |
| BT3 | 5.16 | 80625 |
| BT4 | 5.36 | 71346 |
| BT5 | 4.49 | — |
| BT6 | 6.72 | 62494 |
| BT7 | 7.53 | 49018 |
| BT8 | — | 42262 |
| BT9 | 9.41 | 35668 |
| BT10 | — | 27823 |
| BT11 | 6.18 | 23764 |
| BT12 | 8.72 | 15481 |
| BT13 | 8.14 | 10142 |

As many of these landmarks as possible were identified in each gel image of the dataset. Each feature in the study gels was then assigned a p1 value by linear interpolation or extrapolation (using the MELANIE®-II software) to the two nearest landmarks, and was assigned a MW value by linear interpolation or extrapolation (using the MELANIE®-II software) to the two nearest landmarks.

Matching With Primary Master Image. Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the study. This common image, the “primary master image”, was selected on the basis of protein load (maximum load consistent with maximum feature detection), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

Cross-matching Between Samples. To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Laplacian pyramid). Thus the Laplacian images represent the details in the image at different scales.

To estimate the distortion between any 2 given images, a calculation was performed at level 7 in the pyramid (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of this program was significantly enhanced by the alignment of the images in the manner described above. To improve accuracy still farther, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initializing the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles. After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the BFs, 4) the apparent molecular weight (MW) of the BFs, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to be traced back to an original sample or patient.

Statistical Analysis of the Profiles. A univariate statistical approach was used to analyse the results obtained from the use of the Preferred Technology as specified below to identify BFs from the MCIs within the mastergroup.

(a) The Wilcoxon Rank-Sum test. This test was performed between the normal and the breast cancer samples with specific diagnosis, i.e. ILC-Invasive Lobular Carcinoma, IDC-Invasive Ductal Carcinoma and PE Pleural Effusions. for each MCI basis. The MCIs which recorded a p-value less than or equal to 0.05 were selected as candidate BFs with 95% selectivity.

(b) A further filtering criterion was applied to the candidate BFs emerging from step (a) based on the protein feature presence in the comparison sets. The features with 50% feature presence in the disease and 50% on the normal were selected as the candidate BFs in the second stage filtering

(c) A final filtering criterion was applied to the candidate BFs emerging from step (b) based on an absolute fold change of 2 or greater in the comparison between the normal and the breast cancer samples to select the final candidate BFs.

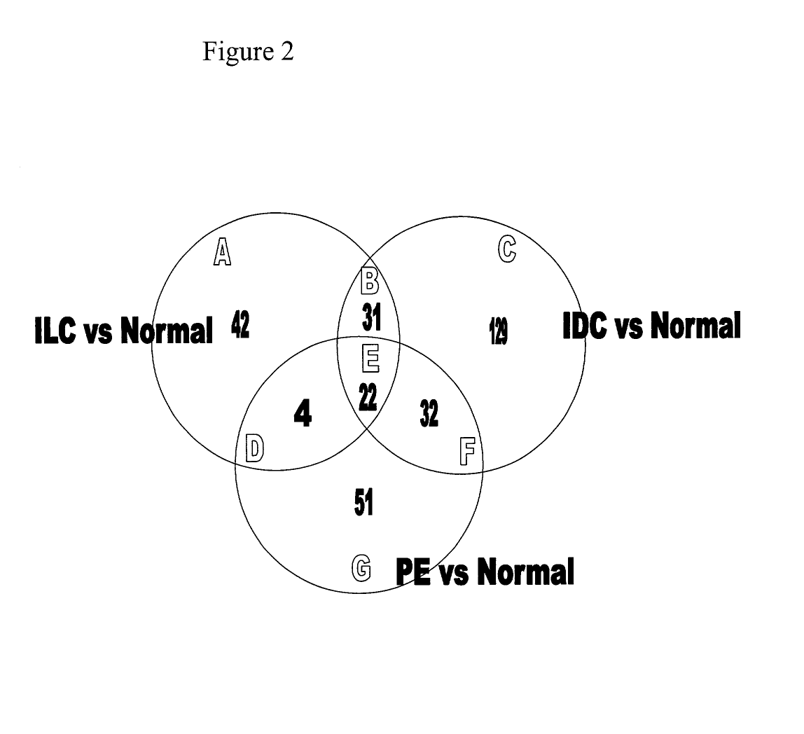

In order to cluster the diagnostic categories, the results were the grouped according to the Venn diagram in FIG. 2. Each Venn diagram position (A, B, C, D, E, F and G) lists a mutually exclusive set of BFs which falls within the diagnostic category.

TABLE XIV
Description of each Venn diagram position and of the
diagnostic category for the BFs listed within

| Table XIV | |
|---|---|
| Venn | |
| Diagram | |
| Position | Diagnostic condition |
| A | ILC specific markers for diagnosing ILC individuals from |
| | the normal |
| B | ILC and IDC specific markers for diagnosing both ILC and |
| | IDC individuals from the normal |
| C | IDC specific markers for diagnosing IDC individuals from |
| | normal |
| D | ILC and PE specific markers for diagnosing both ILC and PE |
| | individuals from the normal |
| E | ILC, IDC and PE specific markers for diagnosing ILC, IDC |
| | and PE individuals from the Normal |
| F | IDC and PE specific makers for diagnosing IDC and PE |
| | individuals from the normal |
| G | PE specific markers for diagnosing PE individuals from the |
| | normal |

Recovery and analysis of selected proteins. Proteins in BFs were robotically excised and processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a nanoflowTM electrospray Z-spray source. For partial amino acid sequencing and identification of BPIs uninterpreted tandem mass spectra of tryptic peptides were searched using the SEQUEST search program (Eng et al., 1994, J. Am. Soc. Mass Spectrom. 5:976-989), version v.C. 1. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662)

Results

Tissues used in this study consisted of 14 samples of reduction mammoplasties carried out for cosmetic reasons from patients in whom no malignancy or pathology was detected; 18 samples of invasive ductal carcinoma; 6 samples of cells sedimented from pleural effusions of patients having metastatic breast cancer; 4 samples of invasive lobular carcinoma. The Venn diagram of FIG. 2 shows the overlap of BFs in different breast cancer states.

These experiments identified 83 features that were decreased and 131 features that were increased in the breast tissue of subjects having invasive ductal carcinoma as compared with the breast tissue of subjects free from breast cancer; 67 features that were decreased and 42 features that were increased in the cells sedimented from pleural effuisions of subjects having metastasized breast cancer as compared with the breast tissue of subjects free from breast cancer; and 38 features that were decreased and 61 features that were increased in the breast tissue of subjects having invasive lobular carcinoma as compared with the breast tissue of subjects free from breast cancer. Details of the BFs present in each position of the Venn diagram together with the associated fold changes and p-values are given in the Tables below.

TABLE XV
Decreased features in Venn Diagram Position A -
ILC specific markers for diagnosing
ILC individuals from the normal:

| Table XV | | | | |
|---|---|---|---|---|
| BF# | pl | MW | Fold Change | p-value |
| BF-85 | 6.32 | 28158 | 3.14 | 0.0039 |
| BF-207 | 5.37 | 50199 | 2.68 | 0.0203 |
| BF-205 | 9.60 | 54437 | 2.67 | |
| BF-204 | 6.33 | 58624 | 2.60 | 0.0172 |
| BF-224 | 4.65 | 18637 | 2.50 | |
| BF-217 | 5.44 | 30182 | 2.22 | 0.0169 |
| BF-25 | 4.63 | 30260 | 2.13 | 0.0035 |
| BF-86 | 5.39 | 29939 | 2.11 | 0.0035 |
| BF-218 | 5.21 | 29877 | 2.10 | |
| BF-225 | 7.63 | 17346 | 2.05 | 0.0171 |
| BF-215 | 5.99 | 36060 | 2.04 | 0.0055 |
| BF-203 | 5.66 | 66584 | 2.01 | |
| BF-208 | 6.66 | 47564 | 2.01 | 0.0126 |

TABLE XVI
Increased features in Venn Diagram Position A -
ILC specific markers for diagnosing
ILC individuals from the normal:

| Table XVI | | | | |
|---|---|---|---|---|
| BF# | pl | MW | Fold Change | p-value |
| BF-226 | 7.89 | 12605 | 35.58 | 0.0369 |
| BF-216 | 6.26 | 32119 | 6.58 | 0.0282 |
| BF-222 | 8.52 | 21327 | 4.08 | 0.0369 |
| BF-201 | 6.10 | 117340 | 2.96 | 0.0227 |
| BF-227 | 5.35 | 11395 | 2.95 | 0.0127 |
| BF-94 | 5.75 | 70640 | 2.75 | 0.0294 |
| BF-212 | 4.69 | 38828 | 2.66 | 0.0127 |
| BF-220 | 4.82 | 21630 | 2.54 | 0.0282 |
| BF-89 | 5.40 | 12234 | 2.48 | 0.0050 |
| BF-112 | 7.72 | 36705 | 2.45 | 0.0155 |
| BF-137 | 6.27 | 32684 | 2.41 | |
| BF-230 | 6.08 | 49018 | 2.37 | 0.0304 |
| BF-223 | 7.09 | 20001 | 2.35 | 0.0127 |
| BF-221 | 6.83 | 21393 | 2.32 | 0.0373 |
| BF-232 | 9.66 | 30698 | 2.32 | |
| BF-219 | 5.24 | 26524 | 2.28 | 0.0346 |
| BF-210 | 8.39 | 43869 | 2.26 | 0.0227 |
| BF-209 | 5.99 | 45443 | 2.22 | 0.0092 |
| BF-228 | 5.70 | 30474 | 2.21 | |
| BF-202 | 5.94 | 97668 | 2.17 | 0.0483 |
| BF-229 | 6.10 | 28798 | 2.17 | |
| BF-231 | 5.85 | 28892 | 2.16 | 0.0227 |
| BF-214 | 5.77 | 36187 | 2.15 | 0.0225 |
| BF-206 | 6.04 | 53124 | 2.15 | 0.0346 |
| BF-76 | 5.04 | 28469 | 2.15 | 0.0063 |
| BF-213 | 8.06 | 39259 | 2.14 | |
| BF-103 | 7.83 | 38361 | 2.03 | 0.0091 |
| BF-92 | 5.97 | 49895 | 2.03 | 0.0223 |
| BF-211 | 6.08 | 40612 | 2.00 | 0.0402 |

TABLE XVII
Decreased features in Venn Diagram Position B -
ILC and IDC specific markers for diagnosing
both ILC and IDC individuals from the normal:

| | | | Fold | | Fold | |
|---|---|---|---|---|---|---|
| Table XVII | | | Change | p-value | Change | p-value |
| BF# | pI | MW | ILC | ILC | IDC | IDC |
| BF-241 | 6.63 | 21542 | 6.17 | 0.0227 | 4.71 | 0.0021 |
| BF-23 | 5.28 | 54631 | 5.69 | 0.0140 | 2.52 | 0.0051 |
| BF-246 | 4.61 | 17035 | 4.31 | 0.0115 | 3.05 | 0.0027 |
| BF-236 | 7.87 | 55046 | 4.27 | 0.0338 | 3.46 | 0.0094 |
| BF-235 | 6.92 | 56452 | 3.71 | | 4.23 | 0.0006 |
| BF-239 | 7.52 | 48780 | 2.88 | 0.0049 | 2.62 | 0.0000 |
| BF-238 | 6.46 | 53651 | 2.75 | | 2.95 | 0.0001 |
| BF-233 | 8.07 | 101343 | 2.27 | 0.0253 | 2.68 | 0.0006 |
| BF-237 | 7.77 | 54027 | 2.25 | 0.0403 | 2.06 | 0.0054 |
| BF-3 | 6.33 | 79913 | 2.21 | 0.0126 | 2.86 | 0.0000 |
| BF-11 | 7.61 | 53837 | 2.19 | 0.0364 | 2.10 | 0.0003 |
| BF-114 | 4.67 | 16800 | 2.14 | 0.0420 | 2.67 | 0.0010 |

TABLE XVIII
Increased features in Venn Diagram Position B -
ILC and IDC specific markers for diagnosing
both ILC and IDC individuals from the normal:

| | | | Fold | | Fold | |
|---|---|---|---|---|---|---|
| Table XVIII | | | Change | p-value | Change | p-value |
| BF# | pI | MW | ILC | ILC | IDC | IDC |
| BF-47 | 4.60 | 37942 | 4.73 | 0.0369 | 6.15 | 0.0011 |
| BF-42 | 4.91 | 40702 | 4.60 | 0.0282 | 3.98 | 0.0004 |
| BF-244 | 5.00 | 40576 | 4.58 | 0.0304 | 4.41 | 0.0025 |
| BF-33 | 4.67 | 47474 | 4.01 | 0.0044 | 4.78 | 0.0000 |
| BF-99 | 6.19 | 41490 | 3.61 | 0.0142 | 2.65 | 0.0057 |
| BF-123 | 5.89 | 34642 | 3.57 | 0.0085 | 2.82 | 0.0008 |
| BF-51 | 5.68 | 70074 | 3.29 | 0.0430 | 2.39 | 0.0014 |
| BF-106 | 8.00 | 23764 | 3.19 | 0.0304 | 2.49 | 0.0064 |
| BF-243 | 6.95 | 18163 | 2.89 | 0.0265 | 2.19 | 0.0451 |
| BF-245 | 8.41 | 27823 | 2.73 | 0.0069 | 2.21 | 0.0030 |
| BF-34 | 7.12 | 32939 | 2.73 | 0.0064 | 2.26 | 0.0037 |
| BF-247 | 6.84 | 13088 | 2.53 | | 2.45 | 0.0485 |
| BF-234 | 5.77 | 96415 | 2.44 | 0.0162 | 2.32 | 0.0003 |
| BF-64 | 4.91 | 21294 | 2.44 | 0.0078 | 2.15 | 0.0120 |
| BF-248 | 4.97 | 15909 | 2.39 | 0.0225 | 3.79 | 0.0028 |
| BF-242 | 7.44 | 19090 | 2.38 | 0.0223 | 2.09 | 0.0132 |
| BF-36 | 5.50 | 22620 | 2.26 | 0.0039 | 2.29 | 0.0000 |
| BF-240 | 5.73 | 39758 | 2.10 | 0.0186 | 2.08 | 0.0045 |
| BF-249 | 5.05 | 150793 | 2.04 | 0.0142 | 2.18 | 0.0221 |

TABLE XIX
Decreased features in Venn Diagram Position C -
IDC specific markers for diagnosing
IDC individuals from normal:

| Table XIX | | | | |
|---|---|---|---|---|
| BF# | pl | MW | Fold Change | p-value |
| BF-268 | 8.39 | 54326 | 7.89 | 0.007 |
| BF-292 | 5.54 | 23266 | 6.43 | 0.014 |
| BF-263 | 8.19 | 56641 | 5.25 | 0.029 |
| BF-265 | 7.57 | 56133 | 5.15 | 0.021 |
| BF-6 | 5.58 | 52905 | 5.10 | 0.000 |
| BF-280 | 6.65 | 35639 | 4.73 | 0.044 |
| BF-260 | 8.59 | 58137 | 4.66 | 0.001 |
| BF-269 | 5.50 | 53757 | 4.46 | 0.001 |
| BF-349 | 7.96 | 54540 | 4.38 | 0.016 |
| BF-271 | 4.78 | 50377 | 4.16 | 0.030 |
| BF-267 | 8.21 | 54623 | 3.73 | 0.034 |
| BF-266 | 6.09 | 54734 | 3.68 | 0.005 |
| BF-295 | 4.71 | 19697 | 3.44 | 0.036 |
| BF-318 | 5.84 | 57392 | 3.25 | 0.003 |
| BF-262 | 8.31 | 56802 | 3.11 | 0.009 |
| BF-12 | 4.91 | 42632 | 3.01 | 0.000 |
| BF-297 | 5.97 | 17794 | 2.99 | 0.014 |
| BF-350 | 4.65 | 23162 | 2.94 | 0.006 |
| BF-319 | 4.83 | 52210 | 2.87 | |
| BF-8 | 5.35 | 53897 | 2.75 | 0.003 |
| BF-291 | 11.61 | 28279 | 2.73 | 0.015 |
| BF-253 | 10.58 | 107058 | 2.68 | 0.006 |
| BF-279 | 10.31 | 37137 | 2.62 | 0.017 |
| BF-270 | 4.95 | 53870 | 2.62 | 0.004 |
| BF-321 | 6.71 | 42419 | 2.59 | 0.001 |
| BF-261 | 5.63 | 57668 | 2.51 | 0.031 |
| BF-283 | 4.51 | 33998 | 2.40 | 0.002 |
| BF-294 | 9.43 | 21085 | 2.40 | 0.048 |
| BF-272 | 7.89 | 49724 | 2.39 | 0.038 |
| BF-20 | 6.84 | 55209 | 2.34 | 0.003 |
| BF-264 | 8.44 | 56704 | 2.28 | 0.001 |
| BF-281 | 8.05 | 35007 | 2.26 | 0.000 |
| BF-290 | 4.97 | 29618 | 2.25 | 0.020 |
| BF-258 | 5.60 | 62015 | 2.22 | 0.011 |
| BF-251 | 7.58 | 112993 | 2.15 | 0.020 |
| BF-254 | 6.29 | 100291 | 2.06 | 0.032 |

TABLE XX
Increased features in Venn Diagram Position C -
IDC specific markers for diagnosing IDC
individuals from normal:

| Table XX | | | | |
|---|---|---|---|---|
| BF# | pl | MW | Fold Change | p-value |
| BF-250 | 7.77 | 118551 | 2.01 | 0.031 |
| BF-303 | 6.49 | 13149 | 2.03 | 0.015 |
| BF-329 | 4.65 | 21268 | 2.06 | 0.005 |
| BF-289 | 6.54 | 29745 | 2.06 | 0.024 |
| BF-344 | 5.96 | 29803 | 2.07 | 0.002 |
| BF-131 | 5.67 | 39910 | 2.09 | 0.002 |
| BF-284 | 5.24 | 33933 | 2.12 | 0.001 |
| BF-335 | 6.09 | 38455 | 2.12 | 0.007 |
| BF-45 | 4.84 | 13610 | 2.13 | 0.002 |
| BF-285 | 7.51 | 32133 | 2.13 | 0.030 |
| BF-288 | 5.03 | 30887 | 2.15 | 0.020 |
| BF-348 | 6.65 | 11445 | 2.16 | 0.032 |
| BF-127 | 4.69 | 37235 | 2.16 | 0.005 |
| BF-339 | 8.17 | 17431 | 2.17 | 0.015 |
| BF-296 | 4.74 | 18536 | 2.19 | 0.031 |
| BF-252 | 6.65 | 109903 | 2.20 | |
| BF-102 | 8.94 | 32089 | 2.20 | 0.046 |
| BF-326 | 6.67 | 29298 | 2.21 | 0.007 |
| BF-62 | 4.54 | 44349 | 2.21 | 0.002 |
| BF-278 | 5.01 | 42154 | 2.22 | 0.004 |
| BF-54 | 6.33 | 31022 | 2.22 | 0.002 |
| BF-287 | 6.35 | 31866 | 2.23 | 0.002 |
| BF-43 | 5.07 | 70174 | 2.23 | 0.000 |
| BF-35 | 6.54 | 34373 | 2.24 | 0.000 |
| BF-259 | 7.40 | 58529 | 2.24 | 0.042 |
| BF-324 | 4.85 | 32089 | 2.25 | 0.022 |
| BF-342 | 4.98 | 58155 | 2.25 | 0.005 |
| BF-309 | 4.91 | 11597 | 2.26 | 0.003 |
| BF-301 | 9.67 | 13895 | 2.29 | 0.011 |
| BF-49 | 6.22 | 12131 | 2.29 | 0.004 |
| BF-311 | 4.59 | 11454 | 2.29 | 0.002 |
| BF-345 | 5.18 | 9650 | 2.33 | 0.042 |
| BF-72 | 6.75 | 24466 | 2.34 | 0.016 |
| BF-293 | 5.40 | 22590 | 2.34 | 0.026 |
| BF-299 | 6.36 | 15927 | 2.35 | 0.006 |
| BF-322 | 8.08 | 41976 | 2.39 | 0.008 |
| BF-41 | 5.46 | 34675 | 2.39 | 0.000 |
| BF-255 | 5.61 | 79733 | 2.40 | 0.027 |
| BF-341 | 5.92 | 12692 | 2.40 | 0.002 |
| BF-320 | 7.89 | 42419 | 2.41 | 0.037 |
| BF-325 | 4.50 | 32089 | 2.41 | 0.009 |
| BF-37 | 4.99 | 30472 | 2.42 | 0.000 |
| BF-71 | 5.39 | 49797 | 2.43 | 0.029 |
| BF-55 | 5.96 | 26272 | 2.47 | 0.024 |
| BF-50 | 5.47 | 23683 | 2.52 | 0.000 |
| BF-316 | 4.86 | 10597 | 2.54 | 0.016 |
| BF-44 | 4.99 | 34791 | 2.55 | 0.001 |
| BF-336 | 7.79 | 36150 | 2.58 | 0.040 |
| BF-300 | 6.56 | 15911 | 2.59 | 0.030 |
| BF-337 | 5.07 | 32640 | 2.64 | 0.008 |
| BF-305 | 5.07 | 11927 | 2.64 | 0.004 |
| BF-340 | 6.55 | 16585 | 2.69 | 0.014 |
| BF-275 | 9.90 | 44863 | 2.70 | 0.020 |
| BF-302 | 4.57 | 13644 | 2.71 | 0.015 |
| BF-48 | 6.97 | 11833 | 2.71 | 0.005 |
| BF-273 | 5.68 | 47695 | 2.76 | 0.036 |
| BF-277 | 6.49 | 43639 | 2.76 | 0.006 |
| BF-125 | 4.78 | 35147 | 2.78 | 0.006 |
| BF-274 | 9.28 | 46617 | 2.85 | 0.022 |
| BF-126 | 7.54 | 13341 | 2.85 | 0.001 |
| BF-338 | 8.05 | 19328 | 2.88 | 0.003 |
| BF-304 | 7.09 | 12765 | 2.92 | 0.046 |
| BF-257 | 5.12 | 69570 | 2.93 | 0.003 |
| BF-256 | 6.97 | 78400 | 2.97 | 0.000 |
| BF-78 | 10.13 | 21133 | 2.99 | 0.030 |
| BF-298 | 4.60 | 16974 | 3.11 | 0.017 |
| BF-122 | 5.25 | 32285 | 3.11 | 0.001 |
| BF-282 | 5.97 | 35101 | 3.12 | 0.004 |
| BF-330 | 5.46 | 20005 | 3.21 | |
| BF-138 | 8.02 | 10932 | 3.22 | 0.005 |
| BF-306 | 6.39 | 11989 | 3.26 | 0.003 |
| BF-310 | 9.64 | 11633 | 3.30 | 0.022 |
| BF-276 | 9.47 | 44888 | 3.40 | 0.005 |
| BF-331 | 7.94 | 17904 | 3.49 | |
| BF-313 | 8.85 | 11298 | 3.55 | 0.023 |
| BF-328 | 5.75 | 22611 | 3.57 | |
| BF-134 | 5.55 | 32551 | 3.61 | 0.008 |
| BF-286 | 6.46 | 31977 | 3.68 | 0.003 |
| BF-343 | 9.25 | 29936 | 3.71 | 0.025 |
| BF-327 | 5.46 | 28516 | 3.72 | |
| BF-315 | 4.56 | 10783 | 3.79 | 0.007 |
| BF-333 | 4.81 | 11957 | 3.83 | 0.004 |
| BF-346 | 6.43 | 31755 | 3.92 | 0.013 |
| BF-323 | 9.13 | 34376 | 3.95 | 0.017 |
| BF-332 | 7.17 | 13562 | 4.27 | 0.009 |
| BF-53 | 4.68 | 41976 | 4.91 | 0.011 |
| BF-334 | 5.52 | 10749 | 5.27 | 0.022 |
| BF-317 | 6.45 | 10483 | 5.75 | 0.000 |
| BF-347 | 7.68 | 28267 | 6.00 | |
| BF-307 | 7.57 | 11987 | 10.68 | 0.000 |
| BF-312 | 8.12 | 11393 | 10.88 | 0.003 |
| BF-314 | 9.70 | 11115 | 22.32 | 0.043 |
| BF-308 | 8.13 | 11900 | 27.14 | 0.048 |

TABLE XXI
Decreased features in Venn Diagram Position D -
ILC and PE specific markers for diagnosing
both ILC and PE individuals from the normal:

| | | | Fold | | Fold | |
|---|---|---|---|---|---|---|
| Table XXI | | | Change | p-value | Change | p-value |
| BF# | pI | MW | ILC | ILC | PE | PE |
| BF-352 | 6.29 | 36762 | 3.06 | | 2.35 | |

TABLE XXII
Increased features in Venn Diagram Position D -
ILC and PE specific markers for diagnosing
both ILC and PE individuals from the normal:

| | | | Fold | | Fold | |
|---|---|---|---|---|---|---|
| Table XXII | | | Change | p-value | Change | p-value |
| BF# | pI | MW | ILC | ILC | PE | PE |
| BF-83 | 6.55 | 58102 | 2.59 | 0.0263 | 2.24 | 0.0138 |
| BF-135 | 6.38 | 25179 | 2.48 | 0.0428 | 2.60 | 0.0131 |
| BF-351 | 4.77 | 40050 | 2.32 | 0.0372 | 3.32 | 0.0402 |

TABLE XXIII
Decreased features in Venn Diagram Position E - ILC, IDC and PE specific
markers for diagnosing ILC, IDC and PE individuals from the Normal:

| | | | Fold | | Fold | | Fold | |
|---|---|---|---|---|---|---|---|---|
| Table XXIII | | | Change | p-value | Change | p-value | Change | p-value |
| BF# | pI | MW | ILC | ILC | IDC | IDC | PE | PE |
| BF-9 | 4.92 | 52046 | 10.27 | | 5.89 | 0.0001 | 3.53 | 0.0085 |
| BF-353 | 7.41 | 55021 | 6.18 | 0.0049 | 4.54 | 0.0000 | 3.80 | 0.0094 |
| BF-2 | 4.85 | 49013 | 5.88 | 0.0106 | 5.08 | 0.0000 | 6.83 | 0.0039 |
| BF-73 | 6.65 | 36425 | 4.85 | 0.0142 | 3.64 | 0.0000 | 7.15 | 0.0058 |
| BF-354 | 8.65 | 54440 | 4.43 | | 7.16 | 0.0127 | 4.07 | 0.0293 |
| BF-15 | 5.49 | 26295 | 4.09 | 0.0049 | 2.09 | 0.0004 | 2.43 | 0.0063 |
| BF-1 | 6.73 | 57363 | 3.88 | 0.0198 | 4.50 | 0.0000 | 4.44 | 0.0035 |
| BF-17 | 6.54 | 54317 | 3.50 | 0.0078 | 2.39 | 0.0023 | 3.03 | 0.0044 |
| BF-355 | 4.80 | 23721 | 3.27 | 0.0068 | 2.55 | 0.0000 | 2.32 | 0.0224 |
| BF-7 | 10.52 | 48758 | 3.01 | 0.0068 | 4.22 | 0.0000 | 2.08 | 0.0233 |
| BF-22 | 7.55 | 21761 | 2.82 | 0.0035 | 2.44 | 0.0000 | 2.58 | 0.0015 |
| BF-356 | 4.85 | 17566 | 2.62 | | 3.02 | 0.0027 | 4.39 | 0.0142 |

TABLE XXIV
Increased features in Venn Diagram Position E - ILC, IDC and PE specific
markers for diagnosing ILC, IDC and PE individuals from the Normal:

| | | | Fold | | Fold | | Fold | |
|---|---|---|---|---|---|---|---|---|
| Table XXIV | | | Change | p-value | Change | p-value | Change | p-value |
| BF# | pI | MW | ILC | ILC | IDC | IDC | PE | PE |
| BF-357 | 7.36 | 11451 | 11.67 | 0.0227 | 20.00 | 0.0011 | 6.52 | 0.0230 |
| BF-52 | 5.89 | 69864 | 7.59 | 0.0162 | 4.51 | 0.0012 | 2.23 | 0.0113 |
| BF-40 | 6.08 | 12362 | 5.97 | 0.0106 | 4.75 | 0.0003 | 2.41 | 0.0222 |
| BF-101 | 5.78 | 68948 | 5.87 | 0.0200 | 3.20 | 0.0122 | 2.15 | 0.0137 |
| BF-60 | 4.50 | 17973 | 5.77 | 0.0304 | 4.37 | 0.0244 | 5.68 | 0.0142 |
| BF-90 | 6.70 | 32475 | 5.15 | 0.0058 | 4.27 | 0.0002 | 2.13 | 0.0133 |
| BF-358 | 4.83 | 41551 | 3.41 | 0.0171 | 2.21 | 0.0008 | 2.86 | 0.0023 |
| BF-38 | 5.16 | 68895 | 3.13 | 0.0142 | 3.24 | 0.0004 | 2.24 | 0.0108 |
| BF-65 | 4.60 | 54421 | 3.04 | 0.0160 | 2.39 | 0.0061 | 2.28 | 0.0327 |
| BF-46 | 5.34 | 29967 | 2.44 | 0.0142 | 2.10 | 0.0010 | 2.66 | 0.0020 |

TABLE XXV
Decreased features in Venn Diagram Position
F - IDC and PE specific makers for diagnosing
IDC and PE individuals from the normal:

| | | | Fold | | Fold | |
|---|---|---|---|---|---|---|
| Table XXV | | | Change | p-value | Change | p-value |
| BF# | pI | MW | PE | PE | IDC | IDC |
| BF-366 | 4.95 | 50611 | 22.26 | 0.0227 | 19.56 | 0.0015 |
| BF-361 | 7.44 | 56631 | 19.27 | 0.0034 | 12.80 | 0.0015 |
| BF-371 | 4.70 | 23016 | 7.96 | | 4.23 | 0.0020 |
| BF-372 | 7.52 | 20516 | 6.09 | 0.0078 | 4.60 | 0.0003 |
| BF-4 | 5.45 | 53541 | 4.77 | 0.0014 | 4.69 | 0.0000 |
| BF-374 | 7.64 | 54567 | 4.22 | 0.0225 | 5.02 | 0.0089 |
| BF-360 | 6.24 | 57140 | 3.87 | 0.0223 | 3.95 | 0.0034 |
| BF-362 | 7.93 | 56475 | 3.59 | 0.0227 | 2.33 | 0.0112 |
| BF-367 | 5.63 | 50779 | 3.37 | 0.0034 | 5.13 | 0.0034 |
| BF-10 | 6.51 | 56205 | 2.72 | 0.0300 | 3.43 | 0.0002 |
| BF-19 | 5.41 | 54116 | 2.63 | 0.0393 | 3.21 | 0.0038 |
| BF-376 | 5.92 | 15600 | 2.60 | 0.0265 | 2.33 | 0.0024 |
| BF-364 | 6.16 | 53276 | 2.60 | 0.0044 | 2.65 | 0.0000 |
| BF-21 | 6.40 | 55957 | 2.56 | 0.0159 | 2.05 | 0.0019 |
| BF-16 | 5.41 | 51333 | 2.38 | 0.0072 | 2.63 | 0.0001 |
| BF-365 | 5.85 | 52603 | 2.31 | 0.0182 | 2.52 | 0.0006 |
| BF-13 | 6.71 | 28131 | 2.31 | 0.0019 | 2.62 | 0.0000 |
| BF-26 | 5.99 | 57554 | 2.27 | 0.0131 | 3.03 | 0.0001 |
| BF-14 | 5.39 | 44908 | 2.25 | 0.0057 | 2.02 | 0.0009 |
| BF-359 | 6.18 | 57756 | 2.24 | 0.0169 | 2.67 | 0.0023 |
| BF-24 | 4.95 | 42165 | 2.23 | 0.0094 | 2.80 | 0.0000 |
| BF-363 | 10.19 | 55984 | 2.09 | 0.0224 | 2.47 | 0.0050 |
| BF-5 | 5.59 | 33775 | 2.08 | 0.0006 | 2.69 | 0.0000 |

TABLE XXVI
Increased features in Venn Diagram Position
F - IDC and PE specific makers for diagnosing
IDC and PE individuals from the normal:

| | | | Fold | | Fold | |
|---|---|---|---|---|---|---|
| Table XXVI | | | Change | p-value | Change | p-value |
| BF# | pI | MW | PE | PE | IDC | IDC |
| BF-373 | 7.16 | 12109 | 6.73 | 0.0298 | 3.85 | 0.0010 |
| BF-375 | 5.58 | 11246 | 3.92 | 0.0227 | 2.94 | 0.0177 |
| BF-377 | 5.74 | 11237 | 3.28 | 0.0200 | 2.32 | 0.0321 |
| BF-369 | 5.34 | 28080 | 3.01 | 0.0304 | 2.86 | 0.0093 |
| BF-39 | 4.77 | 33587 | 2.33 | 0.0006 | 2.07 | 0.0004 |
| BF-57 | 4.68 | 14062 | 2.29 | 0.0180 | 2.07 | 0.0229 |
| BF-67 | 5.40 | 28445 | 2.28 | 0.0282 | 2.04 | 0.0060 |
| BF-370 | 6.84 | 25632 | 2.14 | 0.0085 | 2.23 | 0.0019 |
| BF-368 | 5.10 | 30895 | 2.13 | 0.0182 | 2.56 | 0.0383 |

TABLE XXVII
Decreased features in Venn Diagram Position G - PE specific
markers for diagnosing PE individuals from the normal:

| Table XXVII | | | Fold Change | p-value |
|---|---|---|---|---|
| BF# | pI | MW | PE | PE |
| BF-390 | 5.65 | 53588 | 11.39 | 0.0369 |
| BF-385 | 10.39 | 63571 | 3.57 | 0.0369 |
| BF-27 | 5.73 | 52283 | 3.37 | 0.0253 |
| BF-395 | 4.74 | 48818 | 3.34 | 0.0056 |
| BF-392 | 6.08 | 52448 | 3.23 | 0.0265 |
| BF-415 | 5.79 | 53955 | 3.15 | 0.0043 |
| BF-418 | 10.27 | 28882 | 3.14 | 0.0282 |
| BF-407 | 4.63 | 17126 | 3.11 | |
| BF-420 | 10.22 | 24662 | 2.92 | 0.0304 |
| BF-381 | 8.07 | 74827 | 2.90 | |
| BF-399 | 8.43 | 36084 | 2.77 | 0.0019 |
| BF-75 | 8.13 | 9988 | 2.75 | 0.0078 |
| BF-382 | 8.43 | 74810 | 2.67 | 0.0427 |
| BF-380 | 6.03 | 83606 | 2.66 | 0.0472 |
| BF-421 | 9.49 | 72204 | 2.65 | 0.0298 |
| BF-413 | 8.57 | 10730 | 2.57 | 0.0314 |
| BF-384 | 6.54 | 71457 | 2.53 | |
| BF-400 | 5.70 | 32253 | 2.52 | 0.0282 |
| BF-386 | 6.35 | 62181 | 2.52 | 0.0108 |
| BF-31 | 7.23 | 56167 | 2.49 | 0.0075 |
| BF-383 | 6.24 | 72679 | 2.47 | 0.0150 |
| BF-405 | 4.68 | 17543 | 2.40 | 0.0288 |
| BF-393 | 5.17 | 49168 | 2.33 | 0.0433 |
| BF-412 | 7.00 | 14079 | 2.30 | 0.0098 |
| BF-378 | 6.14 | 127960 | 2.29 | 0.0042 |
| BF-389 | 6.34 | 55702 | 2.24 | 0.0233 |
| BF-388 | 5.66 | 57297 | 2.17 | 0.0109 |
| BF-391 | 6.60 | 53462 | 2.13 | 0.0398 |
| BF-379 | 5.24 | 117050 | 2.09 | 0.0057 |
| BF-398 | 4.68 | 37020 | 2.09 | 0.0454 |
| BF-387 | 5.02 | 58032 | 2.04 | 0.0187 |

TABLE XXVIII
Increased features in Venn Diagram Position G -
PE specific markers for diagnosing PE
individuals from the normal:

| Table XXVIII | | | Fold Change | p-value |
|---|---|---|---|---|
| BF# | pl | MW | PE | PE |
| BF-409 | 4.95 | 15725 | 4.29 | 0.0058 |
| BF-411 | 5.09 | 14959 | 3.60 | 0.0219 |
| BF-408 | 5.69 | 15919 | 3.23 | 0.0227 |
| BF-419 | 9.42 | 21438 | 3.07 | 0.0321 |
| BF-401 | 8.29 | 32300 | 2.98 | 0.0200 |
| BF-396 | 6.89 | 46381 | 2.97 | 0.0130 |
| BF-417 | 5.02 | 31271 | 2.83 | 0.0416 |
| BF-414 | 8.75 | 23225 | 2.82 | 0.0428 |
| BF-410 | 4.60 | 15224 | 2.60 | 0.0039 |
| BF-394 | 5.85 | 48991 | 2.56 | 0.0050 |
| BF-397 | 5.45 | 40176 | 2.55 | 0.0189 |
| BF-403 | 9.31 | 23662 | 2.31 | 0.0219 |
| BF-80 | 7.86 | 82682 | 2.25 | 0.0454 |
| BF-56 | 5.98 | 38417 | 2.13 | 0.0015 |
| BF-81 | 7.29 | 33646 | 2.10 | 0.0252 |
| BF-84 | 5.84 | 50081 | 2.10 | 0.0034 |
| BF-402 | 4.53 | 25717 | 2.08 | 0.0019 |
| BF-416 | 5.10 | 38537 | 2.04 | 0.0200 |
| BF-404 | 7.25 | 22880 | 2.03 | |
| BF-406 | 5.55 | 17447 | 2.00 | |

Each BF was differentially present in the breast tissue of subjects having breast cancer as compared with the breast tissue of subjects free from breast cancer. For some preferred BFs, the difference was highly significant (p<0.01), and for some highly preferred BFs, the difference was still even more significant (p<0.001).

Partial amino acid sequences were determined for the differentially present BPIs in these BFs. Details of these BPIs are provided in Table VII.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

1. A method for diagnosis of breast cancer in a subject, for determining the stage or severity of breast cancer in a subject, for identifying a subject at risk of developing breast cancer, or for monitoring the effect of therapy administered to a subject having breast cancer, said method comprising:
(a) analyzing a test sample of tissue from the subject by two dimensional electrophoresis to generate a two-dimensional array of features, said array comprising at least one chosen feature whose relative abundance correlates with the presence, absence, stage or severity of breast cancer or predicts the onset or course of breast cancer; and
(b) comparing the abundance of each chosen feature in the test sample with the abundance of that chosen feature in body fluid from one or more persons free from breast cancer, or with a previously determined reference range for that feature in subjects free from breast cancer, or with the abundance at least one Expression Reference Feature (ERF) in the test sample.

2. The method of claim 1, wherein the tissue is breast tissue.

3. The method of claim 1, wherein said method is for screening or diagnosis of breast cancer and the relative abundance of at least one chosen feature correlates with the presence or absence of breast cancer.

4. The method of claim 1, wherein said method is for monitoring the effect of therapy administered to a subject having breast cancer and the relative abundance of at least one chosen feature correlates with the severity of breast cancer.

5. The method of claim 2, wherein step (b) comprises comparing the abundance of each chosen feature in the sample with the abundance of that chosen feature in breast tissue from one or more persons free from breast cancer or with a previously determined reference range for that chosen feature in subjects free from breast cancer.

6. The method of claim 1, wherein step (b) comprises quantitatively detecting one or more Breast Cancer-Associated Features (BFs) selected from the group consisting of: BF-1, BF-2, BF-3, BF-4, BF-5, BF-6, BF-7, BF-8, BF-9, BF-10, BF-11, BF-12, BF-13, BF-14, BF-15, BF-16, BF-17, BF-19, BF-20, BF-21, BF-22, BF-23, BF-24, BF-25, BF-26, BF-27, BF-31, BF-33, BF-34, BF-35, BF-36, BF-37, BF-38, BF-39, BF-40, BF-41, BF-42, BF-43, BF-44, BF-45, BF-46, BF-47, BF-48, BF-49, BF-50, BF-51, BF-52, BF-53, BF-54, BF-55, BF-56, BF-57, BF-60, BF-62, BF-64, BF-65, BF-67, BF-71, BF-72, BF-73, BF-75, BF-76, BF-78, BF-80, BF-81, BF-83, BF-84, BF-85, BF-86, BF-89, BF-90, BF-92, BF-94, BF-99, BF-101, BF-102, BF-103, BF-106, BF-112, BF-114, BF-122, BF-123, BF-125, BF-126, BF-127, BF-131, BF-134, BF-135, BF-137, BF-138, BF-201, BF-202, BF-203, BF-204, BF-205, BF-206, BF-207, BF-208, BF-209, BF-210, BF-211, BF-212, BF-213, BF-214, BF-215, BF-216, BF-217, BF-218, BF-219, BF-220, BF-221, BF-222, BF-223, BF-224, BF-225, BF-226, BF-227, BF-228, BF-229, BF-230, BF-231, BF-232, BF-233, BF-234, BF-235, BF-236, BF-237, BF-238, BF-239, BF-240, BF-241, BF-242, BF-243, BF-244, BF-245, BF-246, BF-247, BF-248, BF-249, BF-250, BF-251, BF-252, BF-253, BF-254, BF-255, BF-256, BF-257, BF-258, BF-259, BF-260, BF-261, BF-262, BF-263, BF-264, BF-265, BF-266, BF-267, BF-268, BF-269, BF-270, BF-271, BF-272, BF-273, BF-274, BF-275, BF-276, BF-277, BF-278, BF-279, BF-280, BF-281, BF-282, BF-283, BF-284, BF-285, BF-286, BF-287, BF-288, BF-289, BF-290, BF-291, BF-292, BF-293, BF-294, BF-295, BF-296, BF-297, BF-298, BF-299, BF-300, BF-301, BF-302, BF-303, BF-304, BF-305, BF-306, BF-307, BF-308, BF-309, BF-310, BF-311, BF-312, BF-313, BF-314, BF-315, BF-316, BF-317, BF-318, BF-319, BF-320, BF-321, BF-322, BF-323, BF-324, BF-325, BF-326, BF-327, BF-328, BF-329, BF-330, BF-331, BF-332, BF-333, BF-334, BF-335, BF-336, BF-337, BF-338, BF-339, BF-340, BF-341, BF-342, BF-343, BF-344, BF-345, BF-346, BF-347, BF-348, BF-349, BF-350, BF-351, BF-352, BF-353, BF-354, BF-355, BF-356, BF-357, BF-358, BF-359, BF-360, BF-361, BF-262, BF-363, BF-364, BF-365, BF-366, BF-367, BF-368, BF-369, BF-370, BF-371, BF-372, BF-373, BF-374, BF-375, BF-376, BF-377, BF-378, BF-379, BF-380, BF-381, BF-382, BF-383, BF-384, BF-385, BF-386, BF-387, BF-388, BF-389, BF-390, BF-391, BF-392, BF-393, BF-394, BF-395, BF-396, BF-397, BF-398, BF-399, BF-400, BF-401, BF-402, BF-403, BF-404, BF-405, BF-406, BF-407, BF-408, BF-409, BF-410, BF-411, BF-412, BF-413, BF-414, BF-415, BF-416, BF-417, BF-418, BF-419, BF-420, or BF-421.

7. The method according to claim 1, wherein step (a) comprises isoelectric focussing followed by sodium dodecyl sulfate polyacrylamide gel electrophoresis (SDS-PAGE).

8. A method for screening, diagnosis or prognosis of breast cancer in a subject, for determining the stage or severity of breast cancer in a subject, for identifying a subject at risk of developing breast cancer, or for monitoring the effect of therapy administered to a subject having breast cancer, said method comprising quantitatively detecting, in a sample of breast tissue from the subject, at least one of a Breast Cancer-Associated Protein Isoform (BPI) selected from the group consisting of: BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30 BPI-33, BPI-34, BPI-35, BPI-36, BPI-37, BPI-38, BPI-39, BPI-40, BPI-42, BPI-47, BPI-58, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-269, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380.

9. The method according to claim 8, wherein the step of quantitatively detecting comprises testing at least one aliquot of the sample, said step of testing comprising:
(a) contacting the aliquot with an antibody that is immunospecific for a preselected BPI; and
(b) quantitatively measuring any binding that has occurred between the antibody and at least one species in the aliquot.

10. The method according to claim 9, wherein the antibody is a monoclonal antibody.

11. The method according to claim 9, wherein the step of quantitatively detecting comprises testing a plurality of aliquots with a plurality of antibodies for quantitative detection of a plurality of preselected BPIs.

12. The method according to claim 11, wherein the antibodies are monoclonal antibodies.

13. A preparation comprising an isolated Breast Cancer-Associated Protein Isoform (BPI) selected from the group comprising: BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30, BPI-31, BPI-33, BPI-34, BPI-35, BPI-36, BPI-37, BPI-38, BPI-39, BPI-40, BPI-42, BPI-47, BPI-57, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380.

14. A kit comprising the preparation of claim 13.

15. An antibody capable of immunospecific binding to a Breast Cancer-Associated Protein Isoform (BPI) selected from the group consisting of: BPI-1, BPI-2, BPI-3, BPI-4, BPI-5, BPI-6, BPI-7, BPI-8, BPI-11, BPI-12, BPI-20, BPI-21, BPI-24, BPI-25, BPI-28, BPI-30, BPI-31, BPI-33, BPI-34, BPI-35, BPI-36, BPI-37, BPI-38, BPI-39, BPI-40, BPI-42, BPI-47, BPI-57, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-269, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380.

16. The antibody of claim 15, which is a monoclonal antibody.

17. The antibody of claim 15, which binds to the BPI with greater affinity than to another isoform of the BPI.

18. The antibody of claim 15, which binds to the BPI with greater affinity than to any other isoform of the BPI.

19. A kit comprising the antibody of claim 15.

20. A pharmaceutical composition comprising a therapeutically effective amount of an antibody of claim 15 and a pharmaceutically acceptable carrier.

21. A pharmaceutical composition comprising:
a therapeutically effective amount of a fragment or derivative of an antibody of claim 15, said fragment or derivative containing the binding domain of the antibody; and
a pharmaceutically acceptable carrer.

22. A method of treating or preventing breast cancer comprising administering to a subject in need of such treatment or prevention a therapeutically effective amount of a nucleic acid encoding a Breast Cancer-Associated Protein Isoform (BPI) selected from the group consisting of: BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30, BPI-31, BPI-33, BPI-34, BPI-35, BPI-36, BPI-37, BPI-38, BPI-39, BPI-40, BPI-42, BPI-47, BPI-57, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-269, BPI-270, BPI-271, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380.

23. A method of treating or preventing breast cancer comprising administering to a subject in need of such treatment or prevention a therapeutically effective amount of a nucleic acid that inhibits the function of one or more of a Breast Cancer-Associated Protein Isoform (BPI) selected from the group consisting of: BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30, BPI-31, BPI-33, BPI-34, BPI-35, BPI-36, BPI-37, BPI-38, BPI-39, BPI-40, BPI-42, BPI-47, BPI-57, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-269, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380.

24. The method of claim 23, wherein the nucleic acid is a BPI antisense nucleic acid or ribozyme.

25. A method of screening for agents that interact with a BPI, a BPI fragment, or a BPI-related polypeptide, said method comprising:
(a) contacting a BPI, a biologically active portion of a BPI, or a BPI-related polypeptide with a candidate agent; and
(b) determining whether or not the candidate agent interacts with the BPI, the BPI fragment, or the BPI-related polypeptide.

26. The method of claim 25, wherein the BPI, the BPI fragment, or the BPI-related polypeptide is expressed by cells.

27. The method of claim 25, wherein the cells express a recombinant BPI, a recombinant BPI fragment, or a recombinant BPI-related polypeptide.

28. A method of screening for agents that modulate the expression or activity of a BPI or a BPI-related polypeptide comprising:
(a) contacting a first population of cells expressing a BPI or a BPI-related polypeptide with a candidate agent;
(b) contacting a second population of cells expressing said BPI or said BPI-related polypeptide with a control agent; and
(c) comparing the level of said BPI or said BPI-related polypeptide or mRNA encoding said BPI or said BPI-related polypeptide in the first and second populations of cells, or comparing the level of induction of a cellular second messenger in the first and second populations of cells.

29. The method of claim 28, wherein the level of said BPI or said BPI-related polypeptide, mRNA encoding said BPI or said BPI-related polypeptide, or said cellular second messenger is greater in the first population of cells than in the second population of cells.

30. The method of claim 28, wherein the level of said BPI or said BPI-related polypeptide, mRNA encoding said BPI or said BPI-related polypeptide, or said cellular second messenger is less in the first population of cells than in the second population of cells.

31. A method of screening for or identifying agents that modulate the expression or activity of a BPI or a BPI-related polypeptide comprising:
(a) administering a candidate agent to a first mammal or group of mammals;
(b) administering a control agent to a second mammal or group of mammals; and
(c) comparing the level of expression of the BPI or the BPI-related polypeptide or of mRNA encoding the BPI or the BPI-related polypeptide in the first and second groups, or comparing the level of induction of a cellular second messenger in the first and second groups.

32. The method of claim 31, wherein the mammals are animal models for breast cancer.

33. The method of claim 31, wherein the level of expression of said BPI or said BPI-related polypeptide, mRNA encoding said BPI or said BPI-related polypeptide, or of said cellular second messenger is greater in the first group than in the second group.

34. The method of claim 31, wherein the level of expression of said BPI or said BPI-related polypeptide, mRNA encoding said BPI or said BPI-related polypeptide, or of said cellular second messenger is less than in the first group than in the second group.

35. The method of claim 31, wherein the levels of said BPI or said BPI-related polypeptide, mRNA encoding said BPI or said BPI-related polypeptide, or of said cellular second messenger in the first and second groups are further compared to the level of said BPI or said BPI-related polypeptide or said mRNA encoding said BPI or said BPI-related polypeptide in normal control mammals.

36. The method of claim 31, wherein administration of said candidate agent modulates the level of said BPI or said BPI-related polypeptide, or said mRNA encoding said BPI or said BPI-related polypeptide, or said cellular second messenger in the first group towards the levels of said BPI or said BPI-related polypeptide or said mRNA or said cellular second messenger in the second group.

37. The method of claim 31, wherein said mammals are human subjects having breast cancer.

38. A method of screening for or identifying agents that interact with a BPI or a BPI-related polypeptide, comprising
(a) contacting a candidate agent with the BPI or the BPI-related polypeptide, and
(b) quantitatively detecting binding, if any, between the agent and the BPI or the BPI-related polypeptide.

39. A method of screening for or identifying agents that modulate the activity of a BPI or a BPI-related polypeptide, comprising
(a) in a first aliquot, contacting a candidate agent with the BPI or the BPI-related polypeptide, and
(b) comparing the activity of the BPI or the BPI-related polypeptide in the first aliquot after addition of the candidate agent with the activity of the BPI or the BPI-related polypeptide in a control aliquot, or with a previously determined reference range.

40. The method according to claim 38, wherein the BPI or the BPI-related polypeptide is recombinant protein.

41. The method according to claim 38, wherein the BPI or the BPI-related polypeptide is immobilized on a solid phase.

42. A method for screening, diagnosis or prognosis of breast cancer in a subject or for monitoring the effect of an anti-breast cancer drug or therapy administered to a subject, comprising:
(a) contacting at least one oligonucleotide probe comprising 10 or more consecutive nucleotides complementary to a nucleotide sequence encoding a BPI selected from the group consisting of BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30, BPI-31, BPI-34, BPI-35, BPI-36, BPI-37, BPI-38, BPI-39, BPI-40, BPI-42, BPI-47, BPI-57, BPI-64, BPI-65, BPI-68, BPI-69, BPI-70, BPI-71, BPI-72, BPI-201, BPI-202, BPI-203, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-23 1, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257, BPI-258, BPI-259, BPI-260, BPI-261, BPI-262, BPI-263, BPI-264, BPI-265, BPI-266, BPI-267, BPI-268, BPI-269, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380 with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

43. The method of claim 42, wherein step (a) comprises contacting a plurality of oligonucleotide probes comprising 10 or more consecutive nucleotides complementary to a nucleotide sequence encoding a BPI selected from the group consisting of BPI-1, BPI-2, BPI-3, BPI-4, BPI-5, BPI-6, BPI-7, BPI-8, BPI-11, BPI-12, BPI-20, BPI-21, BPI-23, BPI-24, BPI-25, BPI-28, BPI-30, BPI-31, BPI-33, BPI-34, BPI-35, BPI-36, BPI-37, BPI-38, BPI-39, BPI-40, BPI-42, BPI-47, BPI-57, BPI-58, BPI-64, BPI-65, BPI-68, BPI-69, BPI-70, BPI-71, BPI-72, BPI-201, BPI-202, BPI-203, BPI-204, BPI-205, BPI-206, BPI-207, BPI-208, BPI-209, BPI-210, BPI-211, BPI-212, BPI-213, BPI-214, BPI-215, BPI-216, BPI-217, BPI-218, BPI-219, BPI-220, BPI-221, BPI-222, BPI-223, BPI-224, BPI-225, BPI-226, BPI-227, BPI-228, BPI-229, BPI-231, BPI-232, BPI-233, BPI-234, BPI-235, BPI-236, BPI-237, BPI-238, BPI-239, BPI-240, BPI-241, BPI-242, BPI-243, BPI-244, BPI-245, BPI-246, BPI-247, BPI-248, BPI-249, BPI-250, BPI-251, BPI-252, BPI-253, BPI-254, BPI-255, BPI-256, BPI-257,BPI-258, BPI-259, BPI-260, BPI-261, BPI-262, BPI-263, BPI-264, BPI-265, BPI-266, BPI-267, BPI-268, BPI-269, BPI-270, BPI-271, BPI-272, BPI-273, BPI-274, BPI-276, BPI-277, BPI-278, BPI-279, BPI-280, BPI-281, BPI-282, BPI-283, BPI-301, BPI-302, BPI-303, BPI-304, BPI-306, BPI-308, BPI-309, BPI-311, BPI-312, BPI-313, BPI-314, BPI-315, BPI-316, BPI-317, BPI-318, BPI-319, BPI-320, BPI-321, BPI-322, BPI-323, BPI-324, BPI-325, BPI-326, BPI-327, BPI-328, BPI-329, BPI-330, BPI-331, BPI-332, BPI-333, BPI-334, BPI-335, BPI-336, BPI-337, BPI-338, BPI-339, BPI-340, BPI-341, BPI-342, BPI-343, BPI-344, BPI-346, BPI-347, BPI-348, BPI-349, BPI-350, BPI-352, BPI-353, BPI-354, BPI-355, BPI-356, BPI-357, BPI-358, BPI-359, BPI-360, BPI-361, BPI-362, BPI-363, BPI-364, BPI-365, BPI-366, BPI-367, BPI-368, BPI-369, BPI-370, BPI-371, BPI-372, BPI-374, BPI-376, BPI-378, BPI-379, or BPI-380 with an RNA obtained from a biological sample from the subject or with cDNA copied from the RNA wherein said contacting occurs under conditions that permit hybridization of the probe to the nucleotide sequence if present.

44. The method of claim 42, wherein step (a) includes the step of hybridizing the nucleotide sequence to a DNA array, wherein one or more members of the array are the probes complementary to a plurality of nucleotide sequences encoding distinct BPIs.
